SEQUENCE LISTING (8 sequences)

SEQ ID NO: 1 - protein, 288 aa, Homo sapiens
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - DNA, 867 bp, Homo sapiens
  source 1..867: mol_type = other DNA
atgcagatcccacaggcgccctggccagtcgtctgggcggtgctacaactgggctggcggccaggatggttcttagactccccagacaggccctggaacccccccaccttcttcccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagccgacggccctcggagtgcccagccactgaggcctgaggatggacactgctcttggcccctctga

SEQ ID NO: 3 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - DNA, 873 bp, Homo sapiens
  source 1..873: mol_type = other DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 5 - DNA, 10 bp, Homo sapiens
  source 1..10: mol_type = genomic DNA
acacacacac

SEQ ID NO: 6 - DNA, 23 bp, synthetic construct
  misc_feature 1..23: note = Description of Artificial Sequence: Synthetic oligonucleotide
  source 1..23: mol_type = other DNA
aaaaaaaaaaaaaaaaaaaaaaa

SEQ ID NO: 7 - DNA, 12 bp, synthetic construct
  misc_feature 1..12: note = Description of Artificial Sequence: Synthetic oligonucleotide
  source 1..12: mol_type = other DNA
aaaaaaaaaaaa

SEQ ID NO: 8 - DNA, 10 bp, synthetic construct
  misc_feature 1..10: note = Description of Artificial Sequence: Synthetic oligonucleotide
  source 1..10: mol_type = other DNA
aaaaaaaaaa

(19) United States
(12) Patent Application Publication
(10) Pub. No.: US 2023/0332246 A1
(43) Pub. Date: Oct. 19, 2023

(54) METHODS FOR CHARACTERIZING BIOLOGICAL SAMPLES

(71) Applicants: The General Hospital Corporation, Boston, MA (US); The Hospital for Sick Children, Toronto (CA)

(72) Inventors: Gad GETZ, Boston, MA (US); Yosef MARUVKA, Boston, MA (US); Uri TABORI, Toronto (CA); Jiil CHUNG, Toronto (CA)

(73) Assignees: The General Hospital Corporation, Boston, MA (US); The Hospital for Sick Children, Toronto (CA)

(21) Appl. No.: 18/313,101

(22) Filed: May 5, 2023

Related U.S. Application Data
(63) Continuation of application No. PCT/US2021/058252, filed on Nov. 5, 2021.
(60) Provisional application No. 63/121,181, filed on Dec. 3, 2020.
(60) Provisional application No. 63/111,415, filed on Nov. 9, 2020.
(60) Provisional application No. 63/110,853, filed on Nov. 6, 2020.

(51) Int. Cl. C12Q 1/6886; G16B 20/20; C07K 16/28; A61P 35/00

(52) U.S. Cl. CPC C12Q 1/6886; G16B 20/20; C07K 16/2818; C07K 16/2827; A61P 35/00; C12Q 2600/106; C12Q 2600/156

(57) ABSTRACT

The disclosure features in some aspects methods for identifying subjects with constitutional mismatch repair deficiency (CMMRD), a mismatch repair deficiency (MMD) cancer, a polymerase proofreading deficiency (PPD) cancer, and/or a MMD&PPD cancer. The disclosure also features in some aspects methods for predicting response of a subject to immunotherapy.

### CROSS-REFERENCE TO RELATED APPLICATIONS

This application is a continuation under 35 U.S.C. § 111(a) of PCT International Patent Application No. PCT/US2021/058252, filed Nov. 5, 2021, designating the 10 United States and published in English, which claims priority to and the benefit of U.S. Provisional Application No. 63/121,181, filed Dec. 3, 2020, U.S. Provisional Application No. 63/111,415, filed Nov. 9, 2020, and U.S. Provisional Application No. 63/110,853, filed Nov. 6, 2020, the entire contents of each of which are incorporated herein by reference.

### SEQUENCE LISTING

The present application contains a Sequence Listing which has been submitted electronically in XML format following conversion from the originally filed TXT format.

The content of the electronic XML Sequence Listing, (Date of creation: May 4, 2023; Size: 11,351 bytes; Name: 167741-028007US-Sequence_Listing.xml), and the original TXT format, is herein incorporated by reference in its entirety.

### BACKGROUND

Two major mechanisms that safeguard the fidelity of genome replication are the DNA mismatch repair machinery and DNA polymerase proofreading (c or 8, encoded by POLE and POLD1). Deficiencies of mismatch repair (MMRD) or polymerase proofreading can occur independently or simultaneously, leading to combined DNA replication repair deficiency (RRD). Both mechanisms maintain genome stability by correcting misincorporated non-complementary nucleotides. Thus, when either or both pathways are inactivated, there is an increased rate of replicative mutagenesis that leads to a wide array of hypermutant cancers. The MMR machinery also repairs insertions and deletions in stretches of DNA with small, repeated motifs (called microsatellites), which are created as a result of DNA polymerase slippage. Nevertheless, the impact of the DNA polymerase on the accumulation of these microsatellite insertions and deletions (MS-indels) in human cancer is currently not well-described.

The increased number of MS-indels in MMRD tumors is marked by microsatellite instability (MSI). MSI is clinically used for tumor stratification, referral to germline testing for cancer predisposition, and has recently been approved as a biomarker for immune checkpoint inhibitors (ICI). MSI can be detected by comparing microsatellite lengths of a limited panel of microsatellites (5 loci) between a patient's tumor and germline DNA. The Cancer Genome Atlas (TCGA) used this approach to stratify tumors as microsatellite unstable (MSI-H) or microsatellite stable (MSS) for colorectal, gastric, and endometrial tumors. However, this stratification approach has recently been challenged as being limited in scope, due to the misclassification of some MMRD cancers. Importantly, using this assay, polymerase mutant cancers do not exhibit high MSI and therefore, in humans, the role of polymerase mutations in the accumulation of MS-indels in cancer is currently considered largely unknown.

The most common cause of MMRD in human cancers is somatic hypermethylation of the MLH1 promoter, while others are due to loss of heterozygosity from the germline allele in any one of the MMR genes, MSH2, MSH6, MLH1 and PMS2. Insight regarding the pathogenesis and clinical behavior of RRD can be gained by studying human syndromes with germline mutations in these genes. Lynch syndrome is caused by germline heterozygous inactivating mutations in one of the MMR genes, and is followed by somatic loss of the remaining allele, leading to MMR deficiency. Since it only requires loss of one allele, Lynch syndrome is associated with earlier cancer onset than somatic MMRD. However, although less common, the most aggressive form of MMRD stems from germline biallelic inactivating mutations in one of the MMR genes. This condition, termed constitutional mismatch repair deficiency (CMMRD), is one of the most aggressive cancer syndromes in humans, where virtually all individuals will be affected by a variety of cancers during childhood, and all share hypermutation and specific mutational signatures. Similarly, heterogenous germline polymerase mutations in the exonuclease (proofreading) domain of POLE or POLD1 result in a cancer syndrome termed polymerase proofreading deficiency (PPD), which is less well-described but also leads to hypermutant cancers. MS-indels in either of these cancer predisposition syndromes have not been previously investigated.

In patients with CMMRD, somatic mutations in the polymerase genes result in combined dysfunction of the replication repair machinery, leading to a dramatic accumulation of single nucleotide variations (SNVs). Analysis of SNV-based mutational signatures and their timing also enabled the prediction of early germline events and late treatment-related processes in each cancer that correlate with the type of RRD. Moreover, cancers with both MMRD and PPD have unique SNV signatures that are characteristic of the interaction between the two DNA repair mechanisms. Nevertheless, SNV-based analyses fail to provide information on several biological and clinical questions related to RRD carcinogenesis. These include insights regarding the mechanisms of mutagenesis during RRD tumor initiation and progression, explaining genotype-phenotype correlations between different mutations in MMR and polymerase genes, and the ability to detect RRD in normal cells. These are highly important for the management of these patients, their tumors, and implementation of surveillance to family members.

While indel-based signatures were recently reported, the accumulation of MS-indels and their potential signatures in MMRD during tumorigenesis are not well-described. Furthermore, the impact of polymerase mutations on the accumulation of MS-indels and their corresponding signatures in cancer is not known.

Thus, there remains a need for improved methods and compositions for identification of patients with constitutional mismatch repair deficiency (CMMRD), a mismatch repair deficiency (MMD) cancer, a polymerase proofreading deficiency (PPD) cancer, and/or a MMD&PPD cancer. Further, there remains a need to predict response of patients to immunotherapy.

### SUMMARY OF THE DISCLOSURE

The disclosure features, in some aspects, methods for identifying subjects with constitutional mismatch repair deficiency (CMMRD), a mismatch repair deficiency (MMD) cancer, a polymerase proofreading deficiency (PPD) cancer, and/or a MMD&PPD cancer. The disclosure also features in some aspects methods for predicting response of a subject to immunotherapy.

In one aspect, the invention features a method for characterizing a biological sample from a subject. The method involves calculating MS-sig1 (MMRDness score) and MS-sig2 (POLEness score) that reflect the prevalence of single base insertions and deletions, respectively, in the sample. An increase in either score compared to a reference sample identifies the biological sample as replication repair deficient.

In another aspect, the invention features a method for selecting therapy for a subject having a cancer or tumor. The method involves calculating a POLEness score for a biological sample obtained from the subject, where an increased POLEness score compared to a reference sample selects immune checkpoint inhibitor therapy for the subject, and a decreased or unincreased POLEness score compared to a reference sample indicates that immune checkpoint inhibitor therapy is not appropriate for the subject.

In another aspect, the invention features a method for characterizing a cancer or tumor in a subject. The method involves analyzing sequencing data obtained from a biological sample of the subject to identify microsatellite signatures in the sequencing data and using the identified microsatellite signatures to calculate an MMRDness score, and identifying the cancer as a mismatch repair deficiency (MMRD) cancer if the MMRDness score is elevated compared to a reference sample.

In another aspect, the invention features a method for characterizing a cancer or tumor in a subject. The method involves analyzing sequencing data obtained from a biological sample of the subject to identify microsatellite signatures in the sequencing data and using the identified microsatellite signatures to calculate a POLEness score, and identifying the cancer as a polymerase proofreading deficiency (PPD) cancer if the POLEness score is elevated compared to a reference sample.

In another aspect, the invention features a method for characterizing a cancer or tumor in a subject. The method involves analyzing sequencing data obtained from a biological sample of the subject to identify microsatellite signatures in the sequencing data and using the identified microsatellite signatures to calculate a POLEness score and an MMRDness score, and identifying the cancer or tumor as a replication repair deficiency (RRD) cancer or tumor if the POLEness score and/or the MMRDness score is elevated compared to a reference sample.

In another aspect, the invention features a method of treating a subject having a cancer or tumor. The method involves administering an immune checkpoint inhibitor to the subject, where the subject is selected by calculating a POLEness score for a biological sample obtained from the subject. An increased POLEness score compared to a reference sample selects immune checkpoint inhibitor therapy for the subject.

In any of the above aspects, or embodiments thereof, the score further characterizes the biological sample as mismatch repair proficient (MMRP) if the MMRDness score is not increased, mismatch repair deficient (MMRD) if the MMRDness score is increased, polymerase proof-reading deficient (PPRD/PPD) if the POLEness score is increased, or MMRD&PPD if both the MMRDness and PONEness scores are increased.

In any of the above aspects, or embodiments thereof, the immune checkpoint inhibitor immune therapy involves administering an immune checkpoint inhibitor to the subject. In any of the above aspects, or embodiments thereof, the immune checkpoint inhibitor is a PD-1/PD-L1 inhibitor. In any of the above aspects, or embodiments thereof, the immune checkpoint inhibitor contains an antibody or a fragment thereof. In any of the above aspects, or embodiments thereof, immune checkpoint inhibitor contains nivolumab, pembrolizumab, atezolizumab, durvalumab, and/or avelumab. In any of the above aspects, or embodiments thereof, the immune checkpoint inhibitor contains pembrolizumab.

In any of the above aspects, or embodiments thereof, the calculating involves analyzing sequence data obtained from the biological sample.

In any of the above aspects, or embodiments thereof, the method further involves administering the immune checkpoint inhibitor therapy to the subject with the increased POLEness score.

In any of the above aspects, or embodiments thereof, the biological sample is from a malignant sample. In any of the above aspects, or embodiments thereof, the biological sample is from a non-malignant sample. In any of the above aspects, or embodiments thereof, the biological sample contains cell free DNA. In any of the above aspects, or embodiments thereof, the biological sample is a blood sample or a tissue sample. In embodiments, the tissue sample is from a tumor or neoplasm.

In any of the above aspects, or embodiments thereof, calculating the MMRDness score involves determining in the sequence data the total number of −1 deletions within microsatellite loci with a length of 10-15 bp and dividing by the total number of microsatellite loci with a length of 10-15 bp. In any of the above aspects, or embodiments thereof, the MMRDness score is considered as increased relative to the reference if the MMRDness score is greater than −1.2. In any of the above aspects, or embodiments thereof, the MMRDness score is considered as increased relative to the reference if the MMRDness score is greater than −1.

In any of the above aspects, or embodiments thereof, calculating the POLEness score involves determining in the sequence data the total number of +1 insertions within microsatellite loci with a length of 5-6 bp and dividing by the total number of microsatellite loci with a length of 5-6 bp. In any of the above aspects, or embodiments thereof, the POLEness score is considered as increased relative to the reference if the PONEness score is greater than −3. In any of the above aspects, or embodiments thereof, the POLEness score is considered as increased compared to the reference if the POLEness score is greater than −2.75.

In any of the above aspects, or embodiments thereof, the subject is a pediatric subject. In any of the above aspects, or embodiments thereof, the subject is an adult subject.

In any of the above aspects, or embodiments thereof, the cancer or tumor is a lymphoma, glioma, brain cancer, endometrial cancer, stomach cancer, or colorectal cancer.

In any of the above aspects, or embodiments thereof, the subject has Lynch syndrome.

In any of the above aspects, or embodiments thereof, the sequence data is ultra-low pass coverage sequencing data. In any of the above aspects, or embodiments thereof, the sequence coverage is nonzero and is less than 1×. In any of the above aspects, or embodiments thereof, the sequence coverage is between about 0.1× and about 0.5×. In any of the above aspects, or embodiments thereof, the sequence data is whole-exome sequence data. In any of the above aspects, or embodiments thereof, the sequence data is whole-genome sequence data.

In any of the above aspects, or embodiments thereof, the method does not involve comparing sequence data to a matched normal sample.

In any of the above aspects, or embodiments thereof, the reference sample is from a subject that does not have constitutional mismatch repair deficiency (CMMRD), a subject who is mismatch repair (MMR) proficient, and/or a subject who is polymerase proofreading proficient.

In any of the above aspects, or embodiments thereof, the sequencing data is obtained by sequencing cell free DNA.

In any of the above aspects, or embodiments thereof, the subject does not have a cancer. In any of the above aspects, or embodiments thereof, subject has germline constitutional mismatch repair deficiency.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “amplification” is meant a method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide 5 residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this disclosure it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

By “immune checkpoint inhibitor” is meant an agent that inhibits an immune checkpoint protein.

By “nivolumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “pembrolizumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “atezolizumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “durvalumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “avelumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “PD-1 polypeptide” or “programmed cell death 1 polypeptide” is meant a PD-1 protein or fragment thereof, capable of being activated by PDL-1 and functioning in regulating an immune response and having at least about 85% amino acid sequence identity to GenBank Accession No. AAC51773.1. An exemplary PD-1 amino acid sequence from Homo Sapiens is provided below (GenBank Accession No. AAC51773.1):

(SEQ ID NO: 1)
MQIPQAPWPVVWAVLQLGWRPGWFLDSPDRPWNPPTFFPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

By “PD-1 polynucleotide” is meant a nucleic acid molecule encoding a PD-1 polypeptide, as well as the introns, exons, and regulatory sequences associated with its expression, or fragments thereof. In embodiments, an PD-1 polynucleotide is the genomic sequence, mRNA, or gene associated with and/or required for PD-1 expression. An exemplary PD-1 nucleotide sequence from Homo Sapiens is provided below (GenBank Accession No. U64863.1):

(SEQ ID NO: 2)
ATGCAGATCCCACAGGCGCCCTGGCCAGTCGTCTGGGCGGTGCTACAAC
TGGGCTGGCGGCCAGGATGGTTCTTAGACTCCCCAGACAGGCCCTGGAA
CCCCCCCACCTTCTTCCCAGCCCTGCTCGTGGTGACCGAAGGGGACAAC
GCCACCTTCACCTGCAGCTTCTCCAACACATCGGAGAGCTTCGTGCTAA
ACTGGTACCGCATGAGCCCCAGCAACCAGACGGACAAGCTGGCCGCCTT
CCCCGAGGACCGCAGCCAGCCCGGCCAGGACTGCCGCTTCCGTGTCACA
GCCATCTCCCTGGCCCCCAAGGCGCAGATCAAAGAGAGCCTGCGGGCAG
ACAACTGCCCAACGGGCGTGACTTCCACATGAGCGTGGTCAGGGCCCGG
CGCAATGACAGCGGCACCTACCTCTGTGGGGCTCAGGGTGACAGAGAGA
AGGGCAGAAGTGCCCACAGCCCACCCCAGCCCCTCACCCAGGCCAGCCG
GCCAGTTCCAAACCCTGGTGGTTGGTGTCGTGGGCGGCCTGCTGGGCAG
CCTGGTGCTGCTAGTCTGGGTCCTGGCCGTCATCTGCTCCCGGGCCGCA
CGAGGGACAATAGGAGCCAGGCGCACCGGCCAGCCCCTGAAGGAGGACC
CCTCAGCCGTGCCTGTGTTCTCTGTGGACTATGGGGAGCTGGATTTCCA
GTGGCGAGAGAAGACCCCGGAGCCCCCCGTGCCCTGTGTCCCTGAGCAG
ACGGAGTATGCCACCATTGTCTTTCCTAGCGGAATGGGCACCTCATCCC
CCGCCCGCAGGGGCTCAGCCGACGGCCCTCGGAGTGCCCAGCCACTGAG
GCCTGAGGATGGACACTGCTCTTGGCCCCTCTGA.

By “PDL-1 polypeptide” or “programmed cell death 1 ligand 1” is meant an a PDL-1 protein or fragment thereof, capable of activating PD-1 and having at least about 85% amino acid sequence identity to Genbank Accession No. AAP13470.1. An exemplary PDL-1 amino acid sequence from Homo Sapiens is provided below (GenBank Accession No. AAP13470.1):

(SEQ ID NO: 3)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET.

By “PDL-1 polynucleotide” is meant a nucleic acid molecule encoding a PDL-1 polypeptide, as well as the introns, exons, and regulatory sequences associated with its expression, or fragments thereof. In embodiments, a PDL-1 polynucleotide is the genomic sequence, mRNA, or gene associated with and/or required for PDL-1 expression. An exemplary PDL-1 nucleotide sequence from Homo Sapiens is provided below (GenBank Accession No. AY254342.1):

(SEQ ID NO: 4)
ATGAGGATATTTGCTGTCTTTATATTCATGACCTACTGGCATTTGCTGA
ACGCATTTACTGTCACGGTTCCCAAGGACCTATATGTGGTAGAGTATGG
TAGCAATATGACAATTGAATGCAAATTCCCAGTAGAAAAACAATTAGAC
CTGGCTGCACTAATTGTCTATTGGGAAATGGAGGATAAGAACATTATTC
AATTTGTGCATGGAGAGGAAGACCTGAAGGTTCAGCATAGTAGCTACAG
ACAGAGGGCCCGGCTGTTGAAGGACCAGCTCTCCCTGGGAAATGCTGCA
CTTCAGATCACAGATGTGAAATTGCAGGATGCAGGGGTGTACCGCTGCA
TGATCAGCTATGGTGGTGCCGACTACAAGCGAATTACTGTGAAAGTCAA
TGCCCCATACAACAAAATCAACCAAAGAATTTTGGTTGTGGATCCAGTC
ACCTCTGAACATGAACTGACATGTCAGGCTGAGGGCTACCCCAAGGCCG
AAGTCATCTGGACAAGCAGTGACCATCAAGTCCTGAGTGGTAAGACCAC
CACCACCAATTCCAAGAGAGAGGAGAAGCTTTTCAATGTGACCAGCACA
CTGAGAATCAACACAACAACTAATGAGATTTTCTACTGCACTTTTAGGA
GATTAGATCCTGAGGAAAACCATACAGCTGAATTGGTCATCCCAGAACT
ACCTCTGGCACATCCTCCAAATGAAAGGACTCACTTGGTAATTCTGGGA
GCCATCTTATTATGCCTTGGTGTAGCACTGACATTCATCTTCCGTTTAA
GAAAAGGGAGAATGATGGATGTGAAAAAATGTGGCATCCAAGATACAAA
CTCAAAGAAGCAAAGTGATACACATTTGGAGGAGACGTAA.

Unless specifically stated or obvious from context, as used herein, the term “about” is understood as within a range of normal tolerance in the art. Unless otherwise clear from context, all numerical values provided herein are modified by the term “about.”

The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally, or alternatively, in some embodiments, administration is parenteral. In some embodiments, administration is intravenous.

By “agent” is meant any small compound (e.g., small molecule), antibody, nucleic acid molecule, or polypeptide, or fragments thereof. In one embodiment, an agent of the disclosure is a PD1/PD-L1 immune checkpoint blockade therapy (e.g., immuno-oncology therapeutic).

As used herein, the term “algorithm” refers to any formula, model, mathematical equation, algorithmic, analytical, or programmed process, or statistical technique or classification analysis that takes one or more inputs or parameters, whether continuous or categorical, and calculates an output value, index, index value or score. Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, statistical weights, and neural networks trained on populations or datasets. Also, of use in the context of MSI as described herein are linear and non-linear equations and statistical classification analyses to determine the relationship between the presence of indels detected at specific MS loci in the genome of a subject's tumor sample.

By “ameliorate” is meant decrease, suppress, attenuate, diminish, arrest, or stabilize the development or progression of a disease (e.g., cancer, including cancers having deficiencies of mismatch repair (MMRD) or polymerase proofreading, or combined DNA replication repair deficiency (RRD)).

By “alteration” is meant a change in the expression levels or activity of a gene or polypeptide as detected by standard art known methods such as those described herein. In some embodiments, the alteration is an increase. In some embodiments, the alteration is a 5 decrease. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, colon adenocarcinoma (COAD), esophageal carcinoma (ESCA), rectal adenocarcinoma (READ), stomach adenocarcinoma (STAD) and uterine corpus endometrial carcinoma (UCEC). It is also contemplated within the scope of the disclosure that the techniques herein may be applied to detect MSI in liquid tumors such as, for example, leukemia and lymphoma. In some embodiments, a cancer is associated with

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result. In embodiments, a reference is a subject or a sample from a subject that does not have constitutional mismatch repair deficiency (CMMRD), a subject who is mismatch repair (MMR) proficient, and/or a subject who is polymerase proofreading proficient. In embodiments, the reference is a matched normal sample, where in some instances the matched normal sample is a sample from a healthy subject and/or a subject that does not have constitutional mismatch repair deficiency (CMMRD), a subject who is mismatch repair (MMR) proficient, and/or a subject who is polymerase proofreading proficient.

“Detect” refers to identifying the presence, absence, or amount of the analyte to be detected. In some embodiments, the detection is of POLEness score or MMRDness. 5 By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include cancer (e.g., cancers having deficiencies of mismatch repair (MMRD) or polymerase proofreading, or combined DNA replication repair deficiency (RRD), as well as MSH2 and MLH1 mutant tumors, and/or cancers associated with Lynch Syndrome). In embodiments, the cancer is a lymphoma (e.g., a leukemia), glioma, brain cancer, endometrial cancer, stomach cancer, or colorectal cancer. Further non-limiting examples of diseases include germline constitutional mismatch repair deficiency, other gastrointestinal cancers, and Lynch syndrome.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, an effective amount reduces or stabilizes cell proliferation, cell survival, or increases subject survival.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “homopolymers(s)” is meant a microsatellite (MS) that is a mononucleotide repeat of at least 6 bases (e.g., a stretch of at least 6 consecutive A, C, T or G residues in the DNA). A “homopolymer region” is a MS region in which the microsatellite is a homopolymer. A “homopolymer subregion” refers to a homopolymer microsatellite located within a larger genomic region (e.g., a homopolymer region).

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

As used herein, the term “indel” refers to a mutation in a nucleic acid in which one or more nucleotides are either inserted or deleted, resulting in a net gain or loss of nucleotides that can include any combination of insertions and deletions. Aberrant homopolymer lengths often result from indels.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof.

Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any alteration in a subject or sample that is associated with a disease or disorder. In some embodiments, a marker is POLEness, MMRDness, and others delineated herein. In one embodiment, −1 deletions (MMRDness) or +1 insertions (POLEness) The characteristic patterns of MS-indels and the large number of microsatellites in the genome may enable analyzing tumors without the need for matched normal samples. To test this, scores were designed that reflect the prevalence of MS-sig1 (MMRDness score) and MS-sig2 (POLEness score) in any given sample. Both scores were calculated by taking the logarithm (base 10) of the ratio of the number of −1 deletions (MMRDness) or +1 insertions (POLEness) divided by the total number of MS-loci within the specified lengths (10-15 bp for MMRDness and 5-6 bp for POLEness, Methods). Higher scores indicate higher prevalence of the MMRDness and POLEness signatures in each sample.

As used herein, “microsatellite (MS)” refers to a genetic locus comprising a short (e.g., 1-20, 1-15, 1-10, 1-9, 1-8, 1-7, 1-6,1-5, 1-4, 1-3, 1-2, etc.), tandemly repeated sequence motifs comprising a minimal total length of about 6 bases. A “mononucleotide microsatellite” or refers to a genetic locus comprising a repeated single nucleotide (e.g., poly-A) and is a specific subclass of MSs. A “dinucleotide microsatellite” refers to a genetic locus comprising a motif of two nucleotides that are tandemly repeated, a “trinucleotide microsatellite” refers to a genetic locus comprising three nucleotides that are tandemly repeated, and a “tetranucleotide microsatellite” refers to a genetic locus comprising a motif of four nucleotides that are tandemly repeated. Additional microsatellite motifs can comprise pentanucleotide and hexanucleotide repeats. A “monomorphic microsatellite” is one in which all (or substantially all) individuals, particularly all individuals of a given population, share the same number of repeat units, which is in contrast to a “polymorphic microsatellite,” which is used to refer to microsatellites in which more than about 1% of individuals in a given population display a different number of repeat units in at least of their alleles. When analyzing MS, one may look at genomic DNA of a sample (e.g., genomic DNA of a tumor cell). “Microsatellite region” refers to the genomic context in which a particular microsatellite resides (i.e., the particular genomic region containing the MS).

As used herein, “microsatellite instability (MSI)” refers to a clonal or somatic change in the number of repeated DNA nucleotide units in MSs such as, for example, insertions and deletions (indels). The term “microsatellite stable (MSS)” refers to MSs that do not display a clonal or somatic change in the number of repeated DNA nucleotide units in the respective MSs. In some embodiments detecting MSI in a tumor or cancer cell sample may include classifying MSI or MSS status in the tumor or cancer cell, in which case the method may include a classification step as described herein.

By “neoplasia” is meant a disease or disorder characterized by excess proliferation or reduced apoptosis. Exemplary neoplasias include, for example, cancers having deficiencies of mismatch repair (MMRD) or polymerase proofreading, or combined DNA replication repair deficiency (RRD), as well as MSH2 and MLH1 mutant tumors, Lynch Syndrome).

As used herein, the term “next-generation sequencing (NGS)” refers to a variety of high-throughput sequencing technologies that parallelize the sequencing process, producing thousands or millions of sequence reads at once. NGS parallelization of sequencing reactions can generate hundreds of megabases to gigabases of nucleotide sequence reads in a single instrument run. Unlike conventional sequencing techniques, such as Sanger sequencing, which typically report the average genotype of an aggregate collection of molecules, NGS technologies typically digitally tabulate the sequence of numerous individual DNA fragments (sequence reads discussed in detail below), such that low frequency variants (e.g., variants present at less than about 10%, 5% or 1% frequency in a heterogeneous population of nucleic acid molecules) can be detected. The term “massively parallel” can also be used to refer to the simultaneous generation of sequence information from many different template molecules by NGS. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics,” J Genet Genomics, 201, 38(3): 95-109.

By “reduces” is meant a negative alteration of at least 10%, 25%, 50%, 75%, or 100%.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, the term “subject” includes humans and mammals (e.g., mice, rats, pigs, cats, dogs, horses, and the like). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like. In embodiments, the subject is a pediatric subject. In embodiments, the subject is an adult. In some instances, a pediatric subject is a subject less than 18, 17, 16, 15, 14, 13, 12, 11, 10, 9, 8, 7, 6, 5, 4, 3, 2, 1, 0.5, or 0.1 years of age. In some cases, an adult subject is about or at least about 18, 19, 20, 25, 30, 35, 40, 45, 50, 55, 60, 65, 70, or 75 years of age.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, e.g., arresting its development; and (c) relieving the disease, e.g., causing regression of the disease.

The phrase “pharmaceutically acceptable carrier” is art recognized and includes a pharmaceutically acceptable material, composition, or vehicle, suitable for administering compounds of the present disclosure to mammals. The carriers include liquid or solid filler, diluent, excipient, solvent, or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable salts, esters, amides, and prodrugs” as used herein refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein (e.g., immune checkpoint inhibitors (ICI), such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which [X] may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with [X], in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The term “salts” refers to the relatively non-toxic, inorganic and organic acid addition salts of compounds of the present disclosure. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference.).

A “therapeutically effective amount” of an agent (e.g., immune checkpoint inhibitor) described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts, and scientific literature cited herein are incorporated herein by reference. In addition, the materials, methods, and examples are illustrative only and not intended to be limiting.

These and other embodiments are disclosed or are obvious from and encompassed by the following Detailed Description.

### BRIEF DESCRIPTION OF THE DRAWINGS

FIGS. 1A-1E. Microsatellites Accumulate MS-indels in germline MMRD Cancers. (A) Microsatellite Instability (MSI) status as measured by the Microsatellite Instability Analysis System (MIAS, Promega) of CMMRD samples. Left and right panels: The proportion of MSI high (MSI-H), MSI low (MSI-L) and microsatellite stable (MSS) samples in different tissue types with the number of samples tested (top). The middle section describes a single patient with constitutional mismatch repair deficiency (CMMRD), MMR160, who had a microsatellite stable (MSS) lymphoma and a microsatellite unstable (MSI-H) colorectal cancer. Histograms represent the results of the MIAS, with germline microsatellite lengths of the 5 tested loci (bottom plot) marked as dashed lines in all panels. GI=gastrointestinal. (B) Comparison of exome-wide total microsatellite indel (MS-indel) burden (TMSIB) between pediatric mismatch repair proficient (MMRP) and MMRD cancers, pediatric cancers by tissue type, and between pediatric and adult MMRD cancers (TMSIB in logarithmic scale). Median TMSIB for each group is written beside each box, and sample numbers are written below each group. Statistical significance was calculated by Mann-Whitney U-test. Endo=Endometrial cancers. (C) Proportion of mutated MS-loci in each chromosome in the entire cohort. The average proportion (90.7%) is labelled and shown as a grey dotted line. The height of the bars in the indicate proportion, and similar heights indicate strong correlation of MS-indels to the number of MS-loci in that region of the chromosome. Table 2 shows the similar proportions of MS-loci mutated in each chromosome. (D) Comparison of the number of MS-indels in diagnosis and relapsed pediatric RRD tumors (n=8). Statistical significance was calculated by the Paired-samples Wilcoxon test. (E), left panel, presents an analysis of recurrent indels in microsatellite loci (MS-loci), separated by adult MMRD cancers (upper curve; n=256), and pediatric MMRD tumors (lower curve; n=69). (E), right panel, presents an analysis of recurrent indels in microsatellite loci (MS-loci), separated by adult MMRD colorectal (top curve; n=44), adult MMRD gastric (middle curve; n=70), and pediatric MMRD (bottom curve; n=73). Pediatric MMRD had weaker hotspots. Each point represents the fraction of tumors mutated for a given MS-locus (y-axis) sorted from most- to least-frequently altered (left to right on the x-axis).

FIGS. 2A-2C. MMRD and PPD tumors have distinct microsatellite indel signatures. (A) Comparison of TMSIB between RRD variants (MMRD, PPD, MMRD&PPD) to MMRP in pediatric brain and GI tumors (left) and adult endometrial tumors (right). Sample numbers are written below each group. P values calculated using Mann-Whitney U-test. (B) Graphical presentation of MS-sig1 (top left) and MS-sig2 (top right) based on indel size (x-axis) and microsatellite locus length, separated by A- or C-repeats (z-axis). The total number of MS-sig1 indels and MS-sig2 indels are compared between the RRD variants in boxplots (bottom left and bottom right). P values were calculated using Mann-Whitney U-test. (C) Graphical representation of RRD subgroups(2) separated by age of tumor onset, most common tumor types, proportion of RRD-associated COSMIC SBS signatures, and MS-sigs 1 and 2. The size of each graphic corresponds to the proportion of the cancer observed clinically. MS-sigs correlated to the proportion of the COSMIC SBS signatures in each cluster. Statistical significance was calculated using the Mann-Whitney U-test.

FIGS. 3A-3D. Accumulation of large microsatellite deletions in MMRD cancers towards a preferred final length. (A) Examples of genomic MS-indel heatmaps of germline MMRD and MMRD & PPD cancers in adults (TCGA) and children. Pediatric cancers had an enrichment of single base MS-indels, while adult cancers accumulated insertions of one base and large (>3 bp) deletions that increased in size (x-axis) with increasing locus length (y-axis). Shading represents the log 10 count of MS-indel events. (B) The total number of MS-deletions in both pediatric and adults by locus length (each panel presents a range of 5 bases) and mutation size (x-axis). The number of deletions were averaged for each deletion length. (C) The fraction of MS-indels by initial (x-axis) and final/post-mutation (y-axis) lengths in adult tumors revealing the convergence of MS-loci to 15 bp (indicated arrow). Fraction of indels was calculated by dividing the number of indels corresponding to each initial and mutated locus length by total number of MS-indels in each initial MS-locus length. (D) Schematic representation of the emergence of 15 bp-long microsatellites as a targeted locus length in adult MMRD cancers. Not intending to be bound by theory, the polymerase is prone to slippage after replicating a tract of 15 bp-repeats, resulting in a loop in the template strand upon rebinding. If unrepaired, the loop can result in a deletion that causes the nascent strand to become 15 bp, irrespective of the original locus length. FIG. 3D discloses SEQ ID NO: 6.

FIGS. 4A-4E. Clinical applications of MS-sigs. (A) Two-dimensional plot separating all types of replication repair deficient (RRD) from wildtype (MMRP) cancers using whole genome sequencing. MMRP cancers are represented by the dark grey dots in the lower-left corner of the plot, mismatch repair deficient (MMRD) cancers are represented by the lowermost five light grey dots in the figure, polymerase proofreading deficient (PPD) cancers are represented by the single uppermost dark grey dot, and the remaining light grey dots represent MMRD and PPD cancers. Each dot represents an individual tumor. (B) Discovery of unexpected MMRD tumors using MS-sigs. Two-dimensional plot of presumed MMRP tumors from the Sickkids Cancer Sequencing (KiCS) program. Four tumors from the KiCS cohort that had increased MMRDness are highlighted by arrows. These were later confirmed to be MMRD (Table 5). (C) Detection of germline constitutional mismatch repair deficiency (CMMRD) using ultra low-passage WGS MMRDness scores. Blood samples from CMMRD and relevant control patients were used. Statistical significance was calculated using the Mann-Whitney U-test. (D) Comparison of MS-sigs to other clinically approved (CLIA) methods to diagnose MMRD in normal and malignant cells. Sensitivity was calculated by dividing the number of positive MMRD cases in each assay by the number of samples that were tested. FIG. 4D discloses SEQ ID NOs: 7-8, respectively, in order of appearance. (E) Genotype phenotype differences between different replication repair genes. Comparison of MMRDness between MMR gene mutations, and POLEness in POLEmut vs. POLD1mut cancers. P values were calculated using the Mann-Whitney U-test.

FIGS. 5A-5B. POLEness Score Predicts Response to Immunotherapy in Cancers. (A) Genomic POLEness scores of 22 RRD cancers stratified by response to immune checkpoint inhibition. Overall survival of patients on immunotherapy separated by the median Genomic POLEness scores (median POLEness=−2.92). POLEness=the normalized activity of POLE related signatures. P values for comparing responders to non-responders were calculated using the Mann-Whitney U-test, and Kaplan-Meier curves were generated for survival with p values calculated using the Log Rank test. (B) Same as (A) using exomic POLEness scores from 28 RRD patients (median POLEness=−2.75).

FIGS. 6A-6B. Genotype-specificity and Kinetics of Microsatellite Instability Accumulation in Replication Repair Deficient Cancers. (A) Absence of MSI among deficiencies in each of the four mismatch repair genes. Analysis was done solely in CMMRD brain cancers (n=51) to account for tissue bias. MSI testing was done using the Microsatellite Instability Analysis System (MIAS, Promega). P value was calculated using the Chi-Squared test. (B) Full spectrum of electrophoretograms from the MIAS (Promega) of multiple tumors from a single patient, MMR160. The reference used to determine MSI was the corresponding blood sample, and loci that were different in length by three bases or more from the blood sample were considered significantly unstable. Samples were considered microsatellite instability-high (MSI-H), if three or more loci were significantly mutated, two was considered microsatellite instability-low (MSI-L), and one to zero unstable loci was considered microsatellite stable (MSS). Each colored peak represents an allele of each MS-locus in the panel, which are labelled with the specific length. Grey dashed lines show the reference length for each locus.

FIGS. 7A-7C. Genotype-phenotype Analysis of MS-sigs. (A) MS-sigs 3-5 and their proportions represented in each variant of RRD. The x-axis of the 3D barplot is the size of the indel, ranging from −3 deletions to +3 insertions, the y-axis is the relative proportion of the indels, and the z-axis is the length of the microsatellites from 5-20 bp, with A-repeats on the first half, and C-repeats on the second half. Statistical analyses were done using the Mann-Whitney U test. (B) Comparison of the MS-sig1 and MS-sig2 MS-indels in POLEmut and POLD1mut pediatric RRD cancers. (C) TMSIB between POLEmut cancers and POLD1mut cancers. Statistical analyses were done using the Mann-Whitney U test.

FIG. 8. Heatmaps of Genomic MS-indels in All Replication Repair Deficient Cancers. Genomic MS-indel heatmaps of all adult and pediatric MMRD and combined MMRD&PPD cancers in the study cohort. As seen in the examples in FIG. 3A, adult MMRD cancers (n=27) accumulated deletions that were larger than 3 bp that increase in size (x-axis) with increasing locus length (y-axis). The adult MMRD&PPD cancer (n=1) was the same sample shown in FIG. 3A, and it had a modest increase in +1 base insertions and MS-deletions longer than 3 bases. On the other hand, pediatric MMRD cancers (n=6) almost exclusively had−1 base deletions, and combined MMRD&PPD cases (n=40) had a large increase in +1 base insertions. The patterns seen in the example FIG. 3A could be seen across the entire cohort. Each shade represents the log 10 count of MS-indel events according to the size of the MS-locus and length of the MS-indel.

FIGS. 9A-9F. Quantification and Modeling of Genomic MS-indels in Replication Repair Deficient Cancers. (A) Count of all genomic MS-indels in germline pediatric MMRD (CMMRD, n=6), pediatric RRD (MMRD&PPD, n=40), and adult MMRD endometrial cancers (n=11). The x-axis is the size of each MS-indel, and the y-axis is the total number of each MS-indel. (B) Heatmaps of genomic MS-indels in adult MMRD colorectal cancers (n=12) and pediatric MMRD colorectal cancers (n=4). The larger MS-deletions, which occurred only in the adult colorectal cancers, were similar to the MMRD cohort of all cancer types in FIG. 8, and pediatric colorectal cancers lacked this phenotype. Each shade represents the log 10 sum of the MS-indels at the MS-locus size and MS-indel length. (C) Goodness of Fit (GOF) analysis of adult MMRD cancers to the Stepwise-Mutation Model (SMM) of MS-indel accumulation, represented by the Poisson distribution. The x-axis is the MS-loci lengths, and the y-axis is the GOF score, where lower GOF scores indicate a better fit to the model. (D) Model fit analysis of deletion size (x-axis) at all 11bp (upper panel) and 25 bp (lower panel) MS-loci to the SMM in adult cancers, represented by the Poisson distribution (smooth line). The y-axis indicates the total number of MS-deletions at each indicated size on the x-axis. Close alignment of the points to the Poisson distribution implied resemblance to the SMM. GOF=Goodness of Fit. The lambda value represents the mean deletion size at 11bp- and 25 bp-long loci. (E) The fraction of all mutated microsatellites separated by size in adult MMRD tumors. The x-axis is the size of the mutated microsatellites, and the y-axis represents the fraction of occurrence of the specified microsatellite length. The two peaks occurred at 6 bp and 15 bp, meaning 6 bp and 15 bp were the most common target lengths of microsatellites in these tumors. (F) The fraction of MS-indels by initial (x-axis) and final/post-mutation (y-axis) lengths in pediatric tumors showing the lack of MS-indels larger than 1bp. Fraction of indels was calculated by dividing the number of indels corresponding to each initial and mutated locus length by total number of MS-indels in each initial MS-locus length.

FIGS. 10A-10C. Application of MMRDness and POLEness Scores on diagnosis and classification of germline RRD (A) Ultra-low pass (0.5×) whole-genome sequencing (WGS) MMRDness and POLEness score map of individual RRD tumors (left). MMRDness scores were compared between the replication repair deficient (RRD) subtypes (right). P values were calculated using the Mann-Whitney U-test. (B) Comparison of CMMRD and MMRP cancer MMRDness scores using normalized MMRDness scores, which was calculated by subtracting the MMRDness score of the tumor by the score of the patient-matched non-malignant sample. Arrows indicate the same 4 outliers as FIG. 3B that were validated as MMRD by immunohistochemistry staining. P values were calculated using the Mann-Whitney U-test. (C) Two dimensional plot of MMRDness and POLEness in germline samples sequenced using ultra-low passage sequencing. Even at 0.5× coverage, CMMRD samples had increased MMRDness relative to other RRD types, which had functional MMR systems.

FIGS. 11A-11B. MMRDness and TMB did not predict immunotherapy response. (A) Genomic and Exomic MMRDness compared between responders and non-responders to immune checkpoint inhibition (ICI) showing that there was no correlation between the two groups. P values were calculated using the Mann-Whitney U-test. (B) Tumor mutation burdens (TMB) compared between responders and no-responders to immune checkpoint inhibitor (ICI) showing that there was no correlation between the two groups. P values were calculated using the Mann-Whitney U-test.

FIG. 12. Strand-bias Model of MS-indel Accumulation. Schematic of MS-indel loops created on the parental and nascent strands that result in deletions and insertions in the microsatellites, respectively. Not intending to be bound by theory, the parental strand loops are repaired efficiently by the MMR system, whereas the nascent strand indel-loops are repaired by the proofreading domain of the DNA polymerase; producing the distinct MS-sigs between the two systems of DNA replication repair. Following another cycle of DNA replication, these MS-indels remain permanent in the daughter cells.

The following detailed description, given by way of example, but not intended to limit the disclosure solely to the specific embodiments described, may best be understood in conjunction with the accompanying drawings.

### DETAILED DESCRIPTION OF THE DISCLOSURE

The present disclosure provides, among other things, methods for identifying patients with constitutional mismatch repair deficiency (CMMRD), a mismatch repair deficiency (MMD) cancer, a polymerase proofreading deficiency (PPD) cancer, and/or a MMD&PPD cancer. The present disclosure also provides, among other things, methods for predicting response of a subject to immunotherapy. The methods of the present disclosure can involve using an MMRDness and/or POLEness score to predict response of a subject to immunotherapy.

The invention of the disclosure is based, at least in part, upon discoveries made through the analysis of a large cohort of carefully annotated cancers and normal tissues from patients with germline mutations in the MMR and polymerase genes. As described further in the Examples provide herein, genomic MS-indels from this cohort were analyzed using whole exome and whole genome sequencing, thoroughly investigating the roles of both replication repair machineries in correcting microsatellite alterations, and their impact on cancer progression and response to therapy. Comparison of these tumors to adult RRD cancers and pediatric non-RRD tumors uncovered: (i) important insights into the accumulation of MS-indels in RRD cancers; (ii) a novel and distinct association between polymerase mutations and MS-indel accumulation; and (iii) a potential mechanism of MMRD that produces large deletions in microsatellites by single events that converge to a microsatellite locus length of −15 bp. Using the activity levels of different MS-indel signatures for tumor stratification, genetic diagnosis, and as biomarkers of response to immunotherapy was then considered.

Although replication repair deficiency, either by mismatch repair deficiency (MMRD) and/or loss of DNA polymerase proofreading, can cause hypermutation in cancer, microsatellite instability (MSI) is considered a hallmark of MMRD alone. As described further in the below Examples, by genome-wide analysis of tumors with germline and somatic deficiencies in replication repair, an association between loss of polymerase proofreading and MSI was revealed, especially when both components are lost. Analysis of indels in microsatellites (MS-indels) identified five distinct signatures (MS-sigs). MMRD MS-sigs are dominated by multi-base losses, while mutant-polymerase MS-sigs contain primarily single-base gains. MS-deletions in MMRD tumors depend on the original size of the microsatellite and converge to a preferred length, providing mechanistic insight. Finally, it is demonstrated that MS-sigs can be a powerful clinical tool for managing individuals with germline MMRD and replication repair deficient cancers, as they can detect the replication repair deficiency in normal cells and predict their response to immunotherapy.

Exome- and genome-wide microsatellite instability analysis revealed novel signatures that are uniquely attributed to mismatch repair and DNA polymerase. This provides new mechanistic insight on microsatellite maintenance and can be applied clinically for diagnosis of replication repair deficiency and immunotherapy response prediction.

### Microsatellites

Microsatellites (MSs), also known as short tandem repeats, are regions of the genome characterized by repetition of a short sequence motif (usually 1-6 bp), e.g. AAAAAA or ACACACACAC (SEQ ID NO: 5) (Ellegren, H. Microsatellites: simple sequences with complex evolution. Nat. Rev. Genet. 5, 435-445 (2004)). MSs are abundant in non-transcribed regions of the human genome, but also occur in exons and untranslated regions (UTRs) with a similar frequency. In the germline, rates of insertions and deletions (indels) in MSs are significantly higher than rates of single nucleotide substitutions elsewhere in the genome (10⁻⁴-10⁻³ compared to ˜10⁻⁸ per locus per generation, respectively) (Sun, J. X. et al. A direct characterization of human mutation based on microsatellites. Nat. Genet. 44, 1161-1165 (2012)). The increased indel mutation rate within MSs is thought to arise due to DNA polymerase slippage during replication of repetitive sequences, leading to changes in the number of repeats (Ellegren, H. Microsatellites: simple sequences with complex evolution. Nat. Rev. Genet. 5, 435-445 (2004)). MS indels frequently result in frameshift mutations and can therefore dramatically alter protein function by changing the amino acid sequence and/or introducing premature stop codons (Ellegren, H. Microsatellites: simple sequences with complex evolution. Nat. Rev. Genet. 5, 435-445 (2004)).

Given their prevalence and relatively high mutation rate, it is perhaps not surprising that microsatellites have been widely implicated in human disease. More than 40 hereditary diseases are caused by germline MS indels, including Huntington's disease and fragile X syndrome. In addition, many cancer genes (e.g. TP53, PTEN and NF1) contain MS loci, and in some cases, somatic MS indels have been causally implicated in cancer. Tumors with microsatellite instability (MSI) have dramatically increased numbers of MS indels owing to loss of normal mismatch repair (MMR) function. Although the MSI phenotype has been observed across tumor types, it appears to be most common in colon adenocarcinoma (COAD), stomach adenocarcinoma (STAD), and uterine corpus endometrial carcinoma (UCEC). Given the important prognostic and therapeutic implications of MSI status, many clinical centers perform routine PCR- or immunohistochemistry-based MSI testing for these tumor types.

Despite their potential biological significance, somatic MS indels have not been systematically analyzed in cancer due to challenges associated with their detection via current next-generation sequencing (NGS) technologies. Only NGS reads that span the entire length of a MS and include sufficient 5′ and 3′ flanking sequences can be used to infer the number of repeated motifs in the MS. In addition, the PCR amplification step that is performed during NGS can itself suffer from DNA polymerase slippage events similar to those that lead to MS indels in vivo, thereby creating NGS artifacts that may be falsely interpreted as MS indels. The frequency of such sequencing errors varies across MS loci and depends on parameters such as the specific MS motif and the number of repeats. Therefore, novel methods utilizing principled statistical modeling and noise estimation are required to accurately identify true MS indel events.

Citation or identification of any document in this application is not an admission that such document is available as prior art to the present disclosure.

### Detection of Microsatellite Indels

The present disclosure relates to detecting microsatellite indels in a cancer patient and/or an individual at risk for cancer.

The present disclosure relates to achieving classification by using whole genome or whole exome data. The classification relies both on the fact that high MS instability (MSI-H) contains a large fraction of the MSI loci mutated, as well as the fact that the type of MS indels in MSI-H cases differ from those in microsatellite stable (MSS) cases. For example, MSI tumors tend to have more one-base deletion in medium size loci (8-15 bases), while non-MSI cases, even if they contain many MS indels, have a more uniform ratio of deletions and insertions, and they do not have this bias to medium sized loci. In embodiments, the sequencing is to a coverage of about or at least about 0.001, 0.01, 0.05, 0.1, 0.2, 0.3, 0.4, 0.5, 0.75, 1, 2, 3, 4, 5, 6, 7, 8, 9, or 10×, where a sequencing coverage of 0.01 indicates that a DNA sample has been sequenced such that the amount of DNA sequenced is equivalent in size (e.g., a number of base pair readouts) to about 1% of the genome (e.g., a subject's genome) from which the DNA sample is derived. In embodiments, the sequencing is to a coverage of no more than about 0.001, 0.01, 0.05, 0.1, 0.2, 0.3, 0.4, 0.5, 0.75, 1, 2, 3, 4, 5, 6, 7, 8, 9, or 10×.

The methods of the present disclosure are typically conducted on a biological sample selected from for example tumor biopsy samples, blood samples (isolation and enrichment of shed tumor cells), stool biopsies, sputum, chromosome, pleural fluid, peritoneal fluid, buccal spears or biopsy or urine.

The present disclosure relates to a method of identifying and selecting a subject with a cancer or tumor with high microsatellite instability (MSI-H) (as opposed to low microsatellite instability (MSI-L) or a microsatellite stable (MSS) cancer or tumor) which may comprise detecting a limited plurality of not more than 40, 30, 20 or 10 microsatellite indels associated with the MSI-H cancer or tumor (but not a MSI-L cancer or tumor), in a nucleic acid sample from the subject's cancer or tumor, wherein the limited plurality of not more than 40 or 30 or or 10 microsatellite indels that are highly mutated in MSI (MSI-H) cancers, but have a low indel rate in an MSI-L or MSS cancer or tumor, and/or may be identified by a limited plurality set of indels are selected by MSMuTect, and wherein the subject has an MSI-H cancer or tumor if all or at least 39, 35, 30 or 20 of the 40 of the limited plurality of MS indels is present in the nucleic acid sample from the subject's cancer or tumor.

The diagnostic tests are typically conducted on a biological sample selected from for example tumor biopsy samples, blood samples (isolation and enrichment of shed tumor cells), stool biopsies, sputum, chromosome analysis, pleural fluid, peritoneal fluid, buccal spears, or biopsy or from urine.

The present disclosure also involves other methods for detecting the MS indels of the present disclosure. Whole genome or whole exome sequencing is preferred; however, other methods of sequencing and hybridization are also envisioned.

RNA sequencing (RNA-Seq) is a powerful tool for transcriptome profiling but is hampered by sequence-dependent bias and inaccuracy at low copy numbers intrinsic to exponential PCR amplification. To mitigate these complications to allow truly digital RNA-Seq, a large set of barcode sequences is added in excess, and nearly every cDNA molecule is uniquely labeled by random attachment of barcode sequences to both ends (Shiroguchi K, et al. Proc Natl Acad Sci USA. 2012 Jan. 24; 109(4):1347-52). After PCR, paired-end deep sequencing is applied to read the two barcodes and cDNA sequences. Rather than counting the number of reads, RNA abundance is measured based on the number of unique barcode sequences observed for a given cDNA sequence (Shiroguchi K, et al. Proc Natl Acad Sci USA. 2012 Jan. 24; 109(4):1347-52). The barcodes may be optimized to be unambiguously identifiable, even in the presence of multiple sequencing errors. This method allows counting with single-copy resolution despite sequence-dependent bias and PCR-amplification noise and is analogous to digital PCR but amendable to quantifying a whole transcriptome (Shiroguchi K, et al. Proc Natl Acad Sci U.S.A. 2012 Jan. 24; 109(4):1347-52).

Fixation of cells or tissue may involve the use of cross-linking agents, such as formaldehyde, and may involve embedding cells or tissue in a paraffin wax or polyacrylamide support matrix (Chung K, et al. Nature. 2013 May 16; 497(7449): 322-7).

Amplification may involve thermocycling or isothermal amplification (such as through the methods RPA or LAMP). Cross-linking may involve overlap-extension PCR or use of ligase to associate multiple amplification products with each other.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

Sequencing may be performed on any high-throughput platform with read-length (either single- or paired-end) sufficient to cover both template and cross-linking event UID's. Methods of sequencing oligonucleotides and nucleic acids are well known in the art (see, e.g., WO93/23564, WO98/28440 and WO98/13523; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The present disclosure may be applied to (1) single-cell transcriptomics: cDNA synthesized from mRNA is barcoded and cross-linked during in situ amplification, (2) single-cell proteomics: cDNA or DNA synthesized from RNA- or DNA-tagged antibodies of one or multiple specificities maps the abundance and distributions of different protein-antigens and (3) whole-tissue transcriptomic/proteomic mapping (molecular microscopy or VIPUR microscopy): using the frequency of cross-contamination between cells to determine their physical proximity, and via applications (1) single-cell transcriptomics and (2) single-cell proteomics, determining the global spatial distribution of mRNA, protein, or other biomolecules in a biological sample. This may be used, for example, to screen for anti-cancer immunoglobulins (by analyzing co-localization of B-cells and T-cells within affected tissue) for immunotherapy.

As described in aspects of the disclosure, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

Sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program. % homology may be calculated over contiguous sequences, e.g., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity. However, these more complex methods assign “gap penalties” to each gap that occurs in the alignment so that, for the same number of identical amino acids, a sequence alignment with as few gaps as possible—reflecting higher relatedness between the two compared sequences—may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension. Calculation of maximum % homology therefore first requires the production of an optimal alignment, taking into consideration gap penalties. A suitable computer program for carrying out such an alignment is the GCG Wisconsin Bestfit package (Devereux et al., 1984 Nuc. Acids Research 12 p387). Examples of other software than may perform sequence comparisons include, but are not limited to, the BLAST package (see Ausubel et al., 1999 Short Protocols in Molecular Biology, 4th Ed.—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health). Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244). Once the software has produced an optimal alignment, it is possible to calculate % homology, preferably % sequence identity. The software typically does this as part of the sequence comparison and generates a numerical result.

Embodiments of the disclosure include sequences (both polynucleotide or polypeptide) which may comprise homologous substitution (substitution and replacement are both used herein to mean the interchange of an existing amino acid residue or nucleotide, with an alternative residue or nucleotide) that may occur e.g., like-for-like substitution in the case of amino acids such as basic for basic, acidic for acidic, polar for polar, etc. Non-homologous substitution may also occur e.g., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Hybridization can be performed under conditions of various stringency. Suitable hybridization conditions for the practice of the present disclosure are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present disclosure include any composition detectable by photochemical, biochemical, spectroscopic, immunochemical, electrical, optical, or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, B-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added.

Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylaminolnaphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

The fluorescent label may be a fluorescent protein, such as blue fluorescent protein, cyan fluorescent protein, green fluorescent protein, red fluorescent protein, yellow fluorescent protein or any photoconvertible protein. Colormetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

In an advantageous embodiment, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

The oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the nonnucleic acid detectable moiety.

In some embodiments, a detectable oligonucleotide tag may comprise one or more nonoligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), biotin, DNP (dinitrophenyl), fucose, digoxigenin, haptens, and other detectable moieties known to those skilled in the art. In some embodiments, the detectable moieties may be quantum dots. Methods for detecting such moieties are described herein and/or are known in the art.

Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties.

A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag.

In some embodiments, a detectable oligonucleotide tag may comprise one or more non-oligonucleotide detectable moieties. Examples of detectable moieties include, but are not limited to, fluorophores, microparticles including quantum dots (Empodocles, et al., Nature 399:126-130, 1999), gold nanoparticles (Reichert et al., Anal. Chem. 72:6025-6029, 2000), biotin, DNP (dinitrophenyl), fucose, digoxigenin, haptens, and other detectable moieties known to those skilled in the art. In some embodiments, the detectable moieties are quantum dots. Methods for detecting such moieties are described herein and/or are known in the art.

Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties.

A unique nucleotide sequence may be a nucleotide sequence that is different (and thus distinguishable) from the sequence of each detectable oligonucleotide tag in a plurality of detectable oligonucleotide tags. A unique nucleotide sequence may also be a nucleotide sequence that is different (and thus distinguishable) from the sequence of each detectable oligonucleotide tag in a first plurality of detectable oligonucleotide tags but identical to the sequence of at least one detectable oligonucleotide tag in a second plurality of detectable oligonucleotide tags. A unique sequence may differ from other sequences by multiple bases (or base pairs). The multiple bases may be contiguous or non-contiguous. Methods for obtaining nucleotide sequences (e.g., sequencing methods) are described herein and/or are known in the art.

In some embodiments, detectable oligonucleotide tags comprise one or more of a ligation sequence, a priming sequence, a capture sequence, and a unique sequence (optionally referred to herein as an index sequence). A ligation sequence is a sequence complementary to a second nucleotide sequence which allows for ligation of the detectable oligonucleotide tag to another entity which may comprise the second nucleotide sequence, e.g., another detectable oligonucleotide tag or an oligonucleotide adapter. A priming sequence is a sequence complementary to a primer, e.g., an oligonucleotide primer used for an amplification reaction such as but not limited to PCR. A capture sequence is a sequence capable of being bound by a capture entity. A capture entity may be an oligonucleotide which may comprise a nucleotide sequence complementary to a capture sequence, e.g. a second detectable oligonucleotide tag. A capture entity may also be any other entity capable of binding to the capture sequence, e.g. an antibody, hapten or peptide. An index sequence is a sequence which may comprise a unique nucleotide sequence and/or a detectable moiety as described above.

The present disclosure also relates to a computer system involved in carrying out the methods of the disclosure relating to both computations and sequencing.

### Computer Systems

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., MSIness score) can be displayed on a monitor, display, or other visualizable medium (e.g., a printout). In some embodiments, data or the results of a calculation may be presented in an auditory form.

### Multiplex Assays

The present disclosure also contemplates multiplex assays. The present disclosure is especially well suited for multiplex assays. For example, the disclosure encompasses use of a SureSelect^{XT}′ SureSelect^{XT2} and SureSelect^{QXT} Target Enrichment System for Illumina Multiplexed Sequencing developed by Agilent Technologies (see for example the World Wide Web at (www)agilent.com/genomics/protocolvideos), a SeqCap EZ kit developed by Roche NimbleGen, a TruSeq® Enrichment Kit developed by Illumina and other hybridization-based target enrichment methods and kits that add sample-specific sequence tags either before or after the enrichment step. as well as Illumina HiSeq, MiSeq and NexSeq, Life Technology Ion Torrent. Pacific Biosciences PacBio RSII, Oxford Nanopore MinIon, PromethIon and GridIon and other massively parallel Multiplexed Sequencing PlatformsError! Hyperlink reference not valid.

Usable methods for hybrid selection are described in Melnikov, et al., Genome Biology 12:R73, 2011; Geniez, et al., Symbiosis 58:201-207, 2012; and Matranga, et al., Genome Biology 15:519, 2014). Bait design and hybrid selection was done similarly to a previously published method (see, e.g., Gnirke, et al., Nature biotechnology 27:182-189, 2009, US Patent Publications No. US 2010/0029498, US 2013/0230857, US 2014/0200163, US 2014/0228223, and US 2015/0126377 and International Patent Publication No. WO 2009/099602). Briefly, baits may be designed by first concatenating all consensus sequences (such as LASV) into two single bait sets (such as one for Nigerian clades and another for the Sierra Leone clade). Duplicate probes, defined as a DNA sequence with 0 mismatches, were removed. The baits sequences were tiled across the genome (such as LASV) creating a probe every 50 bases. Two sets of adapters were used for each bait set. Adapters alternated with each 50 base probe to improve the efficiency of PCR amplification of probes. The oligo array was synthesized on a CustomArray B3 Synthesizer, as recommended by the manufacturer. The oligonucleotides were cleaved-off the array and amplified by PCR with primers containing T7 RNA polymerase promoters. Biotinylated baits were then prepared through in vitro transcription (MEGAshortscript, Ambion). RNA baits for each clade were prepared separately and mixed at the equal RNA concentration prior to hybridization. Libraries of the genome (such as LASV) were added to the baits and hybridized over a 72 hrs. After capture and washing, libraries were amplified by PCR using the Illumina adapter sequences. Libraries were then pooled and sequenced on the MiSeq platform.

### Cancer Treatments

Methods of inhibiting and/or treating cancer and tumors in individuals with cancer or a predisposition for developing cancer as identified by methods of the disclosure are also contemplated.

The subject has been diagnosed with cancer or is at risk of developing cancer. The subject is a human, dog, cat, horse, or any animal in which a tumor specific immune response is desired. The tumor is any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas. In an advantageous embodiment, the cancer is an adrenal, breast, cervical, colon, endometrial, rectal or stomach cancer.

The therapeutic agent is for example, a chemotherapeutic agent, radiation, or immunotherapy. Any suitable therapeutic treatment for a particular cancer may be administered. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, tamoxifen, taxol, topotecan hydrochloride, trastuzumab, vinblastine, vincristine and vinorelbine tartrate.

For therapeutic use, administration should begin at the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The disclosure provides compositions for parenteral administration which comprise a solution of the peptides and vaccine compositions are dissolved or suspended in an acceptable carrier, preferably an aqueous carrier. A variety of aqueous carriers may be used, e.g., water, buffered water, 0.9% saline, 0.3% glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents, and the like, for example, sodium acetate, sodium lactate, sodium chloride, potassium chloride, calcium chloride, sorbitan monolaurate, triethanolamine oleate, etc.

In an advantageous embodiment, the cancer therapeutic is an immunotherapeutic. The immunotherapeutic may be a cytokine therapeutic (such as an interferon or an interleukin), a dendritic cell therapeutic or an antibody therapeutic, such as a monoclonal antibody. In a particularly advantageous embodiment, the immunotherapeutic is a neoantigen (see, e.g., U.S. Pat. No. 9,115,402 and US Patent Publication Nos. 20110293637, 20160008447, 20160101170, 20160331822 and 20160339090).

In an advantageous embodiment, treatments for cancer caused by MSI mutations are contemplated. In particular, if the cancer caused by one or more MSI mutations overexpresses a programmed cell death protein 1 (PD-1) receptor ligand, a PD-1 receptor ligand inhibitor may be contemplated as a treatment. Advantageously, the PD-1 receptor ligand is PD-L1. One example of a PD-L1 receptor ligand inhibitor is pembrolizumab (formerly MK-3475 and lambrolizumab, trade name Keytruda) which is known to be efficacious for MSI cancers. The present disclosure encompasses identifying whether a patient has an MSI cancer in order to know whether pembrolizumab would be a good choice for treatment for that patient, e.g., as a companion diagnostic. In particular, identifying of one or more of the mutations in a patient.

Without being bound by theory, pembrolizumab is a therapeutic antibody that binds to and blocks the PD-1, programmed cell death protein 1 located on lymphocytes. This receptor is generally responsible for preventing the immune system from attacking the body's own tissues; by acting as an immune checkpoint. Many cancers make proteins that bind to PD-1, thus shutting down the ability of the body to kill the cancer on its own. Inhibiting PD-1 on the lymphocytes prevents this, allowing the immune system to target and destroy cancer cells. Tumors that have mutations that cause DNA mismatch repair, which often results in microsatellite instability, tend to generate many mutated proteins that could serve as tumor antigens; pembrolizumab appears to facilitate clearance of any such tumor by the immune system, by preventing the self-checkpoint system from blocking the clearance.

In particular, treatments for adrenal, breast, cervical, colon, endometrial, rectal or stomach cancer are especially contemplated.

For adrenal cancer, surgery is recommended to remove the entire adrenal gland. Standard treatment options for adrenocortical carcinoma (ACC) include, but are not limited to, chemotherapy with mitotane, chemotherapy with mitotane plus streptozotocin or mitotane plus etoposide, doxorubicin, and cisplatin, radiation therapy to bone metastases and/or surgical removal of localized metastases, particularly those that are functioning.

For breast cancer, local therapies such as surgery and radiation are recommended. Breast cancer may also be treated systemically by chemotherapy, hormone therapy (such as, but not limited to, tamoxifen, toremifene, fulvestrant or aromatase inhibitors) or targeted therapy (such as, but not limited to, monoclonal antibodies or other therapeutics that target a HER2 protein, a mTor protein or cyclin-dependent kinases, or kinase inhibitors). If the breast cancer is a BRCA cancer, the cancer may be treated and/or prevented by a mastectomy, sapingo-oophorectomy or hormonal therapy medicines, such as selective estrogen receptor modulators or aromatase inhibitors. Hormonal therapy medicines include, but are not limited to, tamoxifen, raloxifene, exemestane or anastrozole.

Cervical cancer may be treated by surgery, radiation, chemotherapy, or targeted therapy (such as an angiogenesis inhibitor). Cervical squamous cell carcinoma may be treated by cryosurgery, laser surgery, loop electrosurgical excision procedure (LEEP/LEETZ), cold knife conization or a simple hysterectomy (as the first treatment or if the cancer returns after other treatments). Endocervical adenocarcinoma (CESC) may be treated by surgery or radiation.

Colon cancer may be treated by surgery or chemotherapy. Some common regimens for treating colon cancer include, but are not limited to: OLFOX: leucovorin, 5-FU, and oxaliplatin (Eloxatin); FOLFIRI: leucovorin, 5-FU, and irinotecan (Camptosar); CapeOX: capecitabine (Xeloda) and oxaliplatin; FOLFOXIRI: leucovorin, 5-FU, oxaliplatin, and irinotecan; One of the above combinations plus either a drug that targets VEGF (bevacizumab [Avastin], ziv-aflibercept [Zaltrap], or ramucirumab [Cyramza]), or a drug that targets EGFR (cetuximab [Erbitux] or panitumumab [Vectibix]); 5-FU and leucovorin, with or without a targeted drug; Capecitabine, with or without a targeted drug; Irinotecan, with or without a targeted drug; Cetuximab alone; Panitumumab alone; Regorafenib (Stivarga) alone; and/or Trifluridine and tipiracil (Lonsurf).

Endometrial cancer may be treated by surgery, chemotherapy, and radiation. Uterine corpus endometrial carcinoma (UCEC) is the most common type of endometrial cancer. Operative procedures used for managing endometrial cancer include the following: exploratory laparotomy, total abdominal hysterectomy, bilateral salpingo-oophorectomy, peritoneal cytology, and pelvic and para-aortic lymphadenectomy. Chemotherapeutic medications such as cisplatin can be used in the management of endometrial carcinoma. Standard treatment options for uterine carcinosarcoma (UCS) include surgery (total abdominal hysterectomy, bilateral salpingo-oophorectomy, and pelvic and periaortic selective lymphadenectomy), surgery plus pelvic radiation therapy, surgery plus adjuvant chemotherapy or surgery plus adjuvant radiation therapy (EORTC-55874).

Rectal cancer may be treated by surgery, chemotherapy, and radiation. Some common regimens for treating rectal cancer include, but are not limited to: FOLFOX: leucovorin, 5-FU, and oxaliplatin (Eloxatin); FOLFIRI: leucovorin, 5-FU, and irinotecan (Camptosar); CapeOX: capecitabine (Xeloda) and oxaliplatin; FOLFOXIRI: leucovorin, 5-FU, oxaliplatin, and irinotecan; One of the above combinations, plus either a drug that targets VEGF (bevacizumab [Avastin], ziv-aflibercept [Zaltrap], or ramucirumab [Cyramza]), or a drug that targets EGFR (cetuximab [Erbitux] or panitumumab [Vectibix]); 5-FU and leucovorin, with or without a targeted drug; Capecitabine, with or without a targeted drug; Irinotecan, with or without a targeted drug; Cetuximab alone; Panitumumab alone; Regorafenib (Stivarga) alone; and/or Trifluridine and tipiracil (Lonsurf).

Stomach cancer may be treated by surgery, radiation, chemotherapy, or targeted therapy (such as a monoclonal antibody or other therapeutics that target a HER2 protein or a VEGF receptor). Drugs approved for stomach cancer include, but are not limited to, Capecitabine (Xeloda). Cisplatin (Platinol), Cyramza (Ramucirumab), Docetaxel, Doxorubicin Hydrochloride, 5-FU (Fluorouracil Injection), Fluorouracil Injection, Herceptin (Trastuzumab), Irinotecan Hydrochloride, Leucovorin Calcium, Mitomycin C, Mitozytrex (Mitomycin C), Mutamycin (Mitomycin C), Ramucirumab, Taxotere (Docetaxel) and Trastuzumab and may be administered individually or in a combination thereof.

The therapeutics of the present disclosure may be delivered in a particle and/or nanoparticle delivery system. Several types of particle and nanoparticle delivery systems and/or formulations are known to be useful in a diverse spectrum of biomedical applications; and particle and nanoparticle delivery systems in the practice of the instant disclosure can be as in WO 2014/093622 (PCT/US13/74667). In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm. As used herein, a particle delivery system/formulation is defined as any biological delivery system/formulation which includes a particle in accordance with the present disclosure. A particle in accordance with the present disclosure is any entity having a greatest dimension (e.g. diameter) of less than 100 microns (μm). In some embodiments, inventive particles have a greatest dimension of less than 10 μm. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the disclosure. In some embodiments, inventive particles have a greatest dimension ranging between 25 nm and 200 nm. Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry(MALDI-TOF), ultraviolet-visible spectroscopy, dual polarisation interferometry and nuclear magnetic resonance (NMR). Characterization (dimension measurements) may be made as to native particles (i.e., preloading) or after loading of the cargo to provide particles of an optimal size for delivery for any in vitro, ex vivo and/or in vivo application of the present disclosure. In certain preferred embodiments, particle dimension (e.g., diameter) characterization is based on measurements using dynamic laser scattering (DLS). Particles delivery systems within the scope of the present disclosure may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present disclosure.

In general, a “nanoparticle” refers to any particle having a diameter of less than 1000 nm. In certain preferred embodiments, nanoparticles of the disclosure have a greatest dimension (e.g., diameter) of 500 nm or less. In other preferred embodiments, nanoparticles of the disclosure have a greatest dimension ranging between 25 nm and 200 nm. In other preferred embodiments, nanoparticles of the disclosure have a greatest dimension of 100 nm or less. In other preferred embodiments, nanoparticles of the disclosure have a greatest dimension ranging between 35 nm and 60 nm. Nanoparticles encompassed in the present disclosure may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present disclosure.

Semi-solid and soft nanoparticles have been manufactured and are within the scope of the present disclosure. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants. Doses of about 5 mg/kg are contemplated, with single or multiple doses, depending on the target tissue. It is mentioned herein experiments involving mice involve 20 g mammals and that dosing can be scaled up to a 70 kg human. With regard to nanoparticles that can deliver RNA, see, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93. Lipid Nanoparticles, Spherical Nucleic Acid (SNA™) constructs, nanoplexes and other nanoparticles (particularly gold nanoparticles) are also contemplate as a means for delivery A recent publication, entitled “In vivo endothelial siRNA delivery using polymeric nanoparticles with low molecular weight” by James E. Dahlman and Carmen Barnes et al. Nature Nanotechnology (2014) published online 11 May 2014, doi:10.1038/nnano.2014.84, incorporated herein in its entirety, showed that polymeric nanoparticles made of low-molecular-weight polyamines and lipids can deliver siRNA to endothelial cells with high efficiency, thereby facilitating the simultaneous silencing of multiple endothelial genes in vivo. The authors reported that unlike lipid or lipid-like nanoparticles, the nanoparticle formulation they used (termed 7C1), differed from traditional lipid-based nanoparticle formulations because it can deliver siRNA to lung endothelial cells at low doses without substantially reducing gene expression in pulmonary immune cells, hepatocytes, or peritoneal immune cells.

### Colorectal Cancer (CRC)

CRC is the third most common cancer type in which about 1.4 million new cases are diagnosed each year. Additionally, CRC results in about 700,000 deaths per year. Unfortunately, the frequency of CRC appears to be increasing throughout the developed world, presumably due to increased risk of CRC associated with alcohol consumption, smoking, obesity, diabetes, the consumption of large amounts of meat, and little physical activity.

About 15% are associated with microsatellite instability (MSI), which can be defined as somatic changes in the length of microsatellites. Based on microsatellite status (e.g., MSI versus MSS), colorectal tumors can be divided into 3 the categories: 1. tumors with high levels of microsatellite instability (MSI-H), 2 tumors with low levels of microsatellite instability (MSI-L), and tumors that are microsatellite stable (MSS).

Lynch syndrome is a hereditary form of autosomal dominant colon cancer that results from inherited mismatch repair gene defects and is characterized by high levels of microsatellite instability and constitutes about 20% of MSI-H CRCs. Lynch Syndrome patients typically display initial cancer onset in their mid-forties, which is in sharp contrast to patients with sporadic MSI-H cancers where the average age is over seventy.

Sporadic MSI-H tumors are usually caused by the epigenetic silencing of MLH1 caused by promoter methylation. Traditionally, Lynch Syndrome tumors are thought to arise from adenomas, while sporadic MSI-H CRCs are believed to arise from serrated polyps. Approximately 80% of MSI-H tumors are sporadic tumors. Sporadic MSI-H tumors are generally predisposed to present in the proximal colon and are more common in women than men.

With respect to CRC, it is therefore clear that the ability to accurately assess MSI status is important because it can define hereditary forms of CRC and inform clinical care. Additionally, identifying patients with Lynch Syndrome is important because they and their relatives have a high risk of developing second primary cancers. Early detection of these cancers has a significant impact upon prognosis, and it has been estimated that more than 60% of Lynch Syndrome cancer deaths could be prevented with proper follow up.

Other exemplary MSI cancers include, but are not limited to, adenocarcinoma (COAD), stomach adenocarcinoma (STAD), and uterine corpus endometrial carcinoma (UCEC).

### MSI Classification

The methods and compositions described herein relate to identification of a new and clinically useful classifier for MSI, the development of which is based upon an assessment of low pass (e.g., about 0.01×) WGS data for a neoplasia or tumor sample. Specific components of the instant MSI classifier include the following.

In embodiments, the methods of the present disclosure involve calculating an MMRDness and/or POLEness score as described herein. In some instances, a biological sample is considered to have an increased MMRDness score if the MMRDness score is greater than (i.e., less negative than) about −1.4, −1.4, −1.3, −1.2, −1.1, −1, −0.9, −0.8, −0.7, −0.6, or −0.5. In some instances, a biological sample is considered to have an increased POLEness score if the POLEness score is greater than (i.e., less negative than) about −3.5, −3.4, −3.3, −3.2, −3.1, −3, −2.9, −2.8, −2.7, −2.6, −2.5, −2.4, −2.3, −2.2, −2.1, −2, −1.9, −1.8, −1.7, −1.6, −1.5, −1.4, −1.3, −1.2, −1.1, or −1.0.

### Reference Sequences

In certain aspects, the instant disclosure provides methods and kits that involve and/or allow for assessment of the presence or absence of one or more sequence variants and/or mutations in a test subject, tissue, cell, or sample, as compared to a corresponding reference sequence. In particular embodiments, a subject, tissue, cell and/or sample is assessed for one or more variants and/or sites of copy number variation within the sequences/sequence locations (e.g., motif A as defined below).

### Amplification and Sequencing Oligonucleotides

In some aspects, WGS or exome sequencing may be performed upon a test sample for purpose of detecting variants and/or copy number variation as described herein and identifying MSI classification and selecting a therapy. In certain embodiments, assessment of candidate and/or test MSI neoplasia or tumor samples can be performed using one or more amplification and/or sequencing oligonucleotides flanking the above-referenced variant sequence and/or copy number variation regions. Design and use of such amplification and sequencing oligonucleotides, and/or copy number detection probes/oligonucleotides, can be performed by one of ordinary skill in the art.

As will be appreciated by one of ordinary skill in the art, any such amplification sequencing and/or copy number detection oligonucleotides can be modified by any of a number of art-recognized moieties and/or exogenous sequences, e.g., to enhance the processes of amplification, sequencing reactions and/or detection. Exemplary oligonucleotide modifications that are expressly contemplated for use with the oligonucleotides of the instant disclosure include, e.g., fluorescent and/or radioactive label modifications; labeling one or more oligonucleotides with a universal amplification sequence (optionally of exogenous origin) and/or labeling one or more oligonucleotides of the instant disclosure with a unique identification sequence (e.g., a “bar-code” sequence, optionally of exogenous origin), as well as other modifications known in the art and suitable for use with oligonucleotides.

### Neural Network Classification

In certain exemplified aspects, a neural network classifier may also be used may be used to define MSI classification groups. As would be appreciated by one of ordinary skill in the art, other forms of classifier (e.g., nearest-neighbor, and various others) can be applied to variant and/or copy number data, to perform such test sample classification.

A neural network consists of units (neurons), arranged in layers, which convert an input vector into some output. Each unit takes an input, applies a function (e.g., a nonlinear function) to it and then passes the output on to the next layer. Generally the networks are defined to be feed-forward: a unit feeds its output to all the units on the next layer, but there is no feedback to the previous layer. Weightings are applied to the signals passing from one unit to another, and it is these weightings which are tuned in the training phase to adapt a neural network to the particular problem at hand. This is the learning phase.

Neural networks have found application in a wide variety of problems. These range from function representation to pattern recognition, with pattern recognition being the focus of use of neural net classifiers of the instant disclosure.

### Clinical Classifier Scoring Algorithm

The techniques herein provide a classifier algorithm to identify neoplasia or tumor samples as either MSI or MSS. The classifier algorithm herein is based, in part, on using high throughput NGS systems to generate sequencing data for as many loci as possible within the neoplasia or tumor, aggregating the WGS data, and applying a weighting system for analysis. For example, if a particular MS locus has 11-15 repeats of the A motif, it may receive a weight score of 1; however, if that particular MS locus does not have 11-15 repeats of the A motif, it will receive a weight score of 0. In this regard, the techniques herein allow generation of indel signature patterns characteristic of either MSI or MSS.

Without being bound by theory, the techniques herein have identified approximately 600,000 loci having 11-15 repeats of the A motif, which means that even if the WGS data for a particular neoplasia or tumor sample has a very low pass coverage of the genome (e.g., 90%-95% of the loci are not covered at all), it will still be sufficient to accurately identify an MSI indel signature pattern and be able to assess the neoplasia or tumor sample as being either MSI or MSS.

It is expressly contemplated that a classifier of the instant disclosure can be used to link discrete genetic signatures, clinical outcome, and specific targeted therapy in clinical trials and in practice. Specifically, it is contemplated that neoplasia or tumors of patients with MSI can be analyzed prospectively with an exemplified classifier or other classifier within the scope of the instant disclosure. The resulting cluster identifications are predictive of the likelihood of response to standard combination chemotherapy and suggest rational targeted therapies based on cluster-specific biology. Additionally, the resulting identifications can determine whether or not a patient is eligible for anti-PDL or anti-PDL1 treatment. It is further expressly contemplated that a classifier of the instant disclosure can also be applied retrospectively to archival tissue from patients on specific clinical trials or therapies.

### Treatment Selection

The methods described herein can be used for selecting, and then optionally administering, an optimal treatment for a subject. Thus the methods described herein include methods for the treatment of cancer, particularly neoplasia or tumors associated with MSI. Generally, the methods include administering a therapeutically effective amount of a treatment as described herein, to a subject who is in need of, or who has been determined to be in need of, such treatment.

As used in this context, to “treat” means to ameliorate at least one symptom of the cancer. For example, a treatment can result in a reduction in tumor size, tumor growth, cancer cell number, cancer cell growth, or metastasis or risk of metastasis.

For example, the methods can include selecting and/or administering a treatment that includes a therapeutically effective amount of an immune checkpoint blocker such as, for example, cytotoxic T-lymphocyte antigen-4 (CTLA-4) and programmed death-1 (PD-1), to a subject having a select MSI tumor or cancer/tumor.

Therapeutic agents specifically implicated for administration in using the instant MSI classifier include inhibitors of the following genetic targets: PD-1 and PD-L1.

### PD-1

The PD-1 receptor-ligand interaction is a major pathway hijacked by tumors to suppress immune control. PD-1, which is expressed on the cell surface of activated T-cells under healthy conditions, normally functions to down-modulate unwanted or excessive immune responses, including autoimmune reactions. The ligands for PD-1 (PD-L1 and PD-L2) are constitutively expressed or can be induced in various tumors. Binding of either PD-L1 or PD-L2 to PD-1 inhibits T-cell activation triggered through the T-cell receptor.

PD-L1 is expressed at low levels on various non-hematopoietic tissues, most notably on vascular endothelium, whereas PD-L2 protein is only detectably expressed on antigen-presenting cells found in lymphoid tissue or chronic inflammatory environments. PD-L2 is thought to control immune T-cell activation in lymphoid organs, whereas PD-L1 serves to dampen unwarranted T-cell function in peripheral tissues. Although healthy organs express little (if any) PD-L1, a variety of cancers were demonstrated to express abundant levels of this T-cell inhibitor. High expression of PD-L1 on tumor cells (and to a lesser extent of PD-L2) has been found to correlate with poor prognosis and survival in various cancer types, including renal cell carcinoma (RCC), pancreatic carcinoma, hepatocellular carcinoma, ovarian carcinoma, and non-small cell lung cancer (NSCLC). Furthermore, PD-1 has been suggested to regulate tumor-specific T cell expansion in patients with malignant MEL. The observed correlation of clinical prognosis with PD-L1 expression in multiple cancers suggests that the PD-1/PD-L1 pathway plays a role in tumor immune evasion and should be considered as an attractive target for therapeutic intervention.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications, or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity, and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Combination Treatments

The compositions and methods of the present disclosure may be used in the context of a number of therapeutic or prophylactic applications. In order to increase the effectiveness of a treatment with the compositions of the present disclosure, e.g., a PD-1/PD-L1 inhibitor selected and/or administered as a single agent, or to augment the protection of another therapy (second therapy), it may be desirable to combine these compositions and methods with one another, or with other agents and methods effective in the treatment, amelioration, or prevention of diseases and pathologic conditions, for example, neoplasia or tumors identified as MSI.

Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

### Pharmaceutical Compositions

Agents of the present disclosure can be incorporated into a variety of formulations for therapeutic use (e.g., by administration) or in the manufacture of a medicament (e.g., for treating or preventing a MSI tumor or cancer with, for example, PD-1/PD-L1 inhibitors) by combining the agents with appropriate pharmaceutically acceptable carriers or diluents, and may be formulated into preparations in solid, semi-solid, liquid, or gaseous forms. Examples of such formulations include, without limitation, tablets, capsules, powders, granules, ointments, solutions, suppositories, injections, inhalants, gels, microspheres, and aerosols.

For example, MSI neoplasia or tumors described herein may be treated with therapeutic agents such as, for example, immunotherapeutic agents that act by effectively stimulating the immune response, e.g., PD-1/PD-L1 inhibitors (e.g., Pembrolizumab). Pembrolizumab is a humanized monoclonal antibody that blocks the interaction between PD-1 and its ligands, PD-L1 and PD-L2. Pembrolizumab is an IgG4 kappa immunoglobulin with an approximate molecular weight of 149 kDa. Pembrolizumab is believed to have a mechanism of action in which binding of the PD-1 ligands, PD-L1 and PD-L2, to the PD-1 receptor found on T cells, inhibits T cell proliferation and cytokine production. Upregulation of PD-1 ligands occurs in some tumors and signaling through this pathway can contribute to inhibition of active T-cell immune surveillance of tumors. Pembrolizumab binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

Programmed cell death 1 (PD-1) and programmed death ligand 1 (PD-L1) blockade as a potential form of cancer immunotherapy are based on the fact that activation of the PD-1/PD-L1 axis serves as a mechanism for tumor evasion of host tumor antigen-specific T-cell immunity. Accordingly, inhibition of PD-1/PDL-1 interaction (and corresponding downstream signaling events) strengthen tumor antigen-specific T-cell responses and corresponding tumor antigen-specific T-cell immunity. Other FDA approved PD-1/PD-L1 immunotherapeutic inhibitors include Nivolumab, which like Pembrolizumab, is a PD-1 inhibitor antibody, and Atezolizumab, Durvalumab, and Avelumab, which are all PD-L1 inhibitor antibodies.

In addition to immunotherapeutic treatments, the invention of the disclosure includes treatment with additional agents, either alone or in combination with the immunotherapeutic treatment (such as the anti-PD-1/PDL-1 therapeutic agent). Examples of such agents include chemotherapeutic agents including chemotherapeutic alkylating agents such as Cyclophosphamide, Mechlorethamine, Chlorambucil, Melphalan, Monofunctional alkylators, Dacarbazine, nitrosoureas, and Temozolomide (Oral dacarbazine); anthracyclines such as Daunorubicin, Doxorubicin, Epirubicin, Idarubicin, Mitoxantrone, Valrubicin, cytoskeletal disruptor agents (taxanes) such as Paclitaxel, Docetaxel, Abraxane and Taxotere; Epothilones; Histone deacetylase inhibitors such as Vorinostat and Romidepsin; topoisomerase I inhibitors such as Irinotecan and Topotecan; topoisomerase II inhibitors such as Etoposide, Teniposide, and Tafluposide; Kinase inhibitors such as Bortezomib, Erlotinib, Gefitinib, Imatinib, Vemurafenib, and Vismodegib; nucleotide analogs and precursor analog agents such as Azacitidine, Azathioprine, Capecitabine, Cytarabine, Doxifluridine, Fluorouracil, Gemcitabine, Hydroxyurea, Mercaptopurine, Methotrexate, and Tioguanine (formerly Thioguanine); peptide antibiotics such as Bleomycin and Actinomycin; Platinum-based agents such as Carboplatin, Cisplatin, Oxaliplatin; Retinoids such as Retinoids, Tretinoin, Alitretinoin, Bexarotene; Vinca alkaloids and derivatives such as Vinblastine, Vincristine, Vindesine and Vinorelbine; as well as other chemotherapeutic agents including all-trans retinoic acid, Docetaxel, Doxifluridine, Epothilone, Fluorouracil, Methotrexate, and Pemetrexed.

A chemotherapeutic agents drugs for use with the invention of the disclosure include any chemical compound used in the treatment of a proliferative disorder. Chemotherapeutic agents include, but are not limited to, RAF inhibitors (e.g., BRAF inhibitors), MEK inhibitors, PI3K inhibitors and AKT inhibitors. Other chemotherapeutic agents include, without being limited to, the following classes of agents: nitrogen mustards, e.g., cyclophosphamide, trofosfamide, ifosfamide and chlorambucil; nitroso ureas, e.g., carmustine (BCNU), lomustine (CCNU), semustine (methyl CCNU) and nimustine (ACNU); ethylene imines and methyl-melamines, e.g., thiotepa; folic acid analogs, e.g., methotrexate; pyrimidine analogs, e.g., 5-fluorouracil and cytarabine; purine analogs, e.g., mercaptopurine and azathioprine; vinca alkaloids, e.g., vinblastine, vincristine and vindesine; epipodophyllotoxins, e.g., etoposide and teniposide; antibiotics, e.g., dactinomycin, daunorubicin, doxorubicin, epirubicin, bleomycin a2, mitomycin c and mitoxantrone; estrogens, e.g., diethyl stilbestrol; gonadotropin-releasing hormone analogs, e.g., leuprolide, buserelin and goserelin; antiestrogens, e.g., tamoxifen and aminoglutethimide; androgens, e.g., testolactone and drostanolonproprionate; platinates, e.g., cisplatin and carboplatin; and interferons, including interferon-alpha, beta and gamma.

Chemotherapeutic agents include, for example, RAF inhibitors (e.g. Vemurafenib or Dabrafenib), MEK inhibitors, PI3K inhibitors, or AKT inhibitors. The RAF inhibitor is, for example, a BRAF inhibitor. The chemotherapeutic agents can be administered alone or in combination (e.g., RAF inhibitors with MEK inhibitors). The cancer is any cancer in which the tumor has a B-RAF activating mutation. For example the cancer is melanoma, colon cancer, lung cancer, brain cancer, hematologic cancers, or thyroid cancer.

In addition, these modulatory agents can also be administered in combination therapy with, e.g., chemotherapeutic agents, hormones, antiangiogens, radiolabeled, compounds, or with surgery, cryotherapy, and/or radiotherapy. The preceding treatment methods can be administered in conjunction with other forms of conventional therapy (e.g., standard-of-care treatments for cancer well known to the skilled artisan), either consecutively with, pre- or post-conventional therapy.

The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer (e.g., a hematological cancer, such as DLBCL), being treated, the combined use of immunotherapeutic agent (e.g., anti-PD1/PDL1), the extent of the disease and other factors familiar to the physician of skill in the art and can be determined by the physician.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients, and the like. The compositions can also include additional substances to approximate physiological conditions, such as pH adjusting and buffering agents, toxicity adjusting agents, wetting agents, and detergents.

Further examples of formulations that are suitable for various types of administration can be found in Remington's Pharmaceutical Sciences, Mace Publishing Company, Philadelphia, PA, 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds (e.g., FDA-approved compounds) of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, e.g. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein may be used (e.g., administered to an individual, such as a human individual, in need of treatment with a PD-1/PD-L1 inhibitor, etc.) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., a BCL2 inhibitor, PI3K inhibitor, BCR/TLR signaling inhibitor, JAK/STAT inhibitor, etc.) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

FDA-approved drugs provided herein are typically formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a PD-1/PD-L1 inhibitor, etc.) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

The FDA-approved drug or other therapy is administered to the subject in an amount sufficient to achieve a desired effect at a desired site (e.g., reduction of cancer size, cancer cell abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a PD-1/PD-L1 inhibitor, etc.) described herein. In certain embodiments, a dose described herein includes independently between 1 mg and 3 mg, inclusive, of an agent (e.g., a PD-1/PD-L1 inhibitor, etc.) described herein. In certain embodiments, a dose described herein includes independently between 3 mg and 10 mg, inclusive, of an agent (e.g., a PD-1/PD-L1 inhibitor, etc.) described herein. In certain embodiments, a dose described herein includes independently between 10 mg and 30 mg, inclusive, of an agent (e.g., a PD-1/PD-L1 inhibitor, etc.) described herein. In certain embodiments, a dose described herein includes independently between 30 mg and 100 mg, inclusive, of an agent (e.g., a PD-1/PD-L1 inhibitor, etc.) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., a PD-1/PD-L1 inhibitor, etc.) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. in a subject or cell. In certain embodiments, a pharmaceutical composition described herein including an agent (e.g., a PD-1/PD-L1 inhibitor, etc.) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an antioxidant, an anti-inflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, chemotherapeutic agents, other epigenetic modifier inhibitors, etc., other anti-cancer agents, immunomodulatory agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising an agent (e.g., a PD-1/PD-L1 inhibitor, etc.) of this disclosure and/or may contain agents (e.g., oligonucleotide primers, probes, etc.) for identifying a cancer or subject as possessing one or more variant sequences. In some embodiments, the kits further include instructions for use in accordance with the methods of this disclosure. In some embodiments, these instructions comprise a description of administration of the agent to treat or diagnose, e.g., a neoplasia or tumor having MSI, according to any of the methods of this disclosure. In some embodiments, the instructions comprise a description of how to detect a MSI class of cancer, for example in an individual, in a tissue sample, or in a cell.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a class of MSI cancer, in a subject. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In certain embodiments, at least one active agent in the composition is a PD-1/PD-L1 inhibitor, an epigenetic modifier, an epigenetic modifier inhibitor, etc. The container may further comprise a second pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: A Distinct Pattern of Microsatellite Instability in CMMRD Cancers

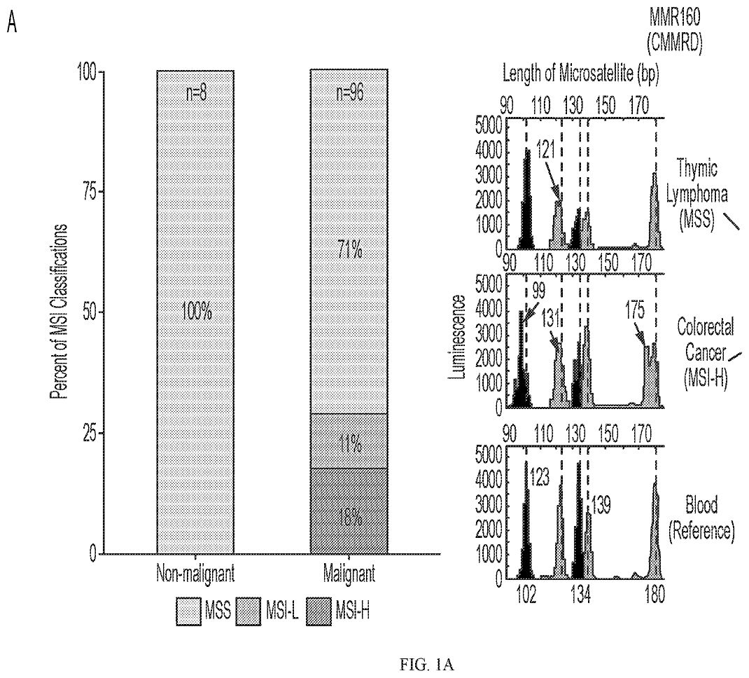

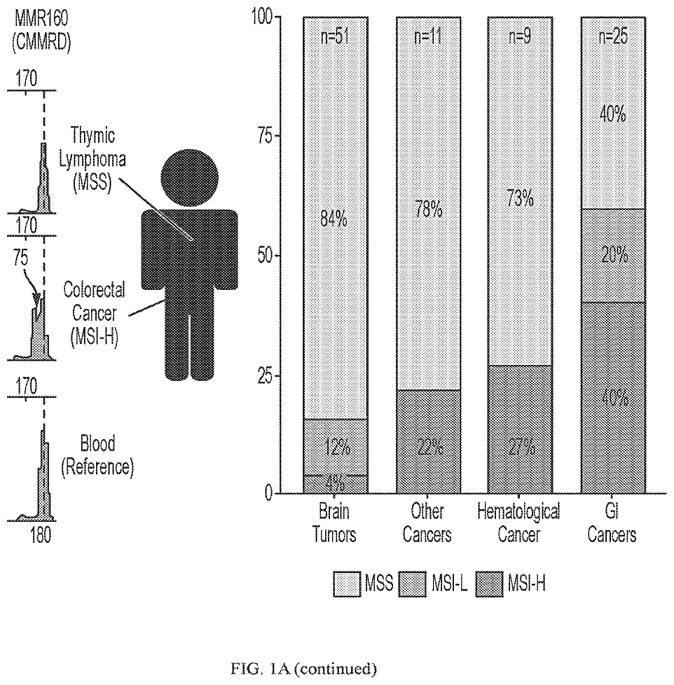

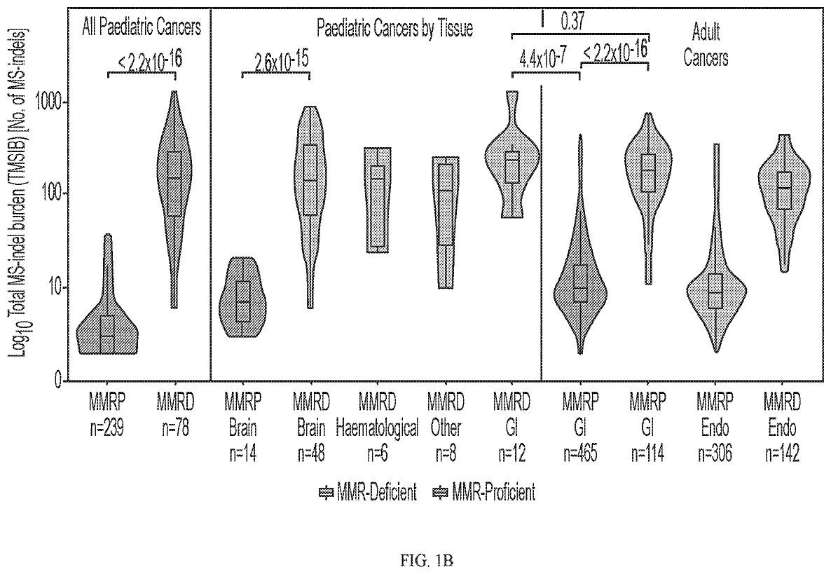

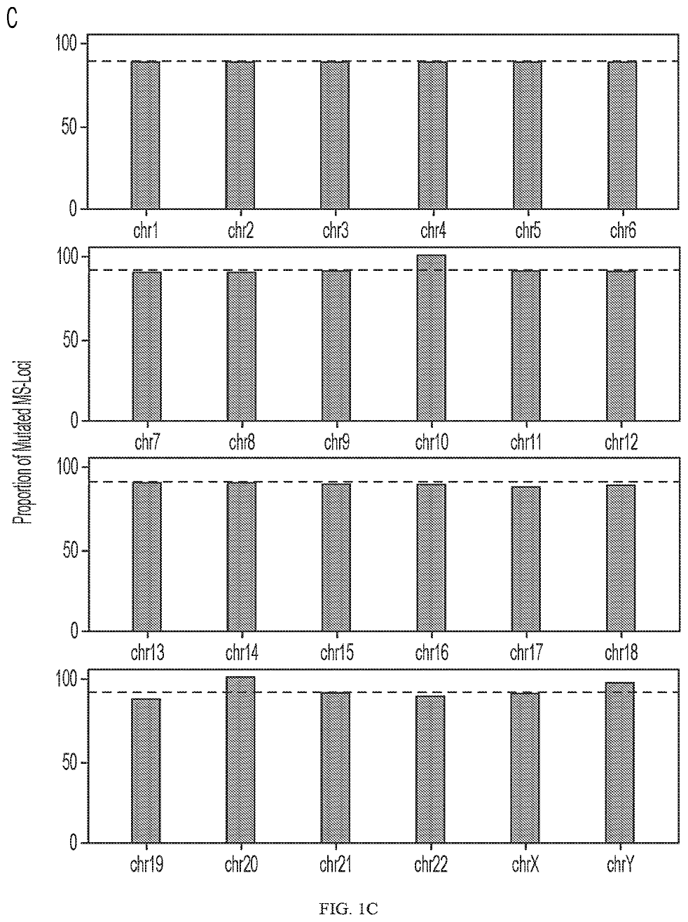

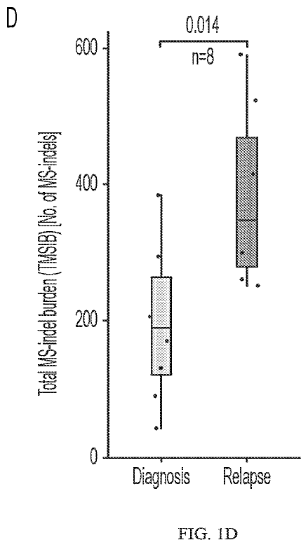

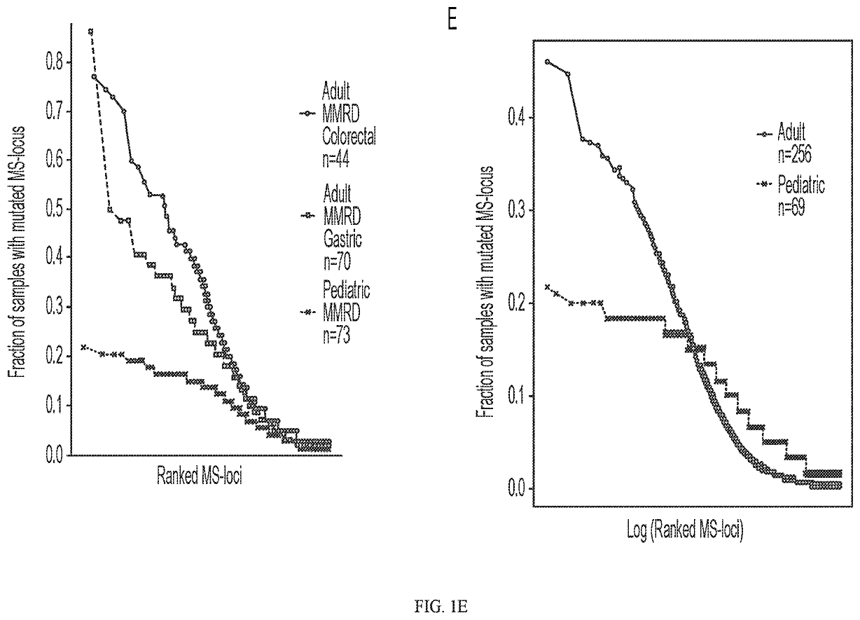

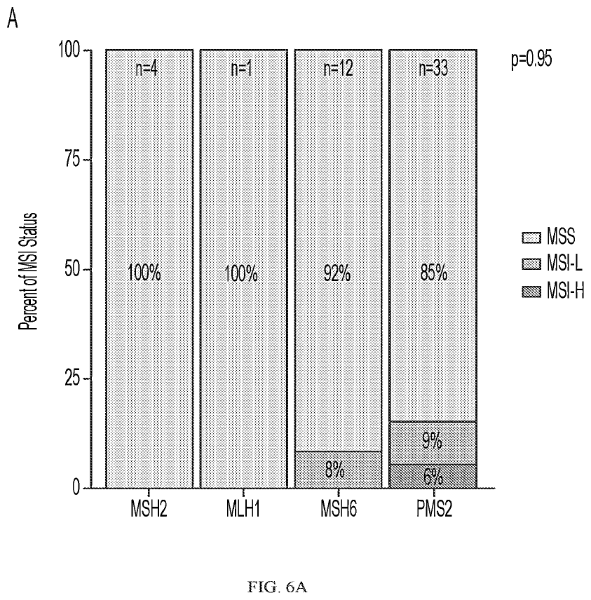

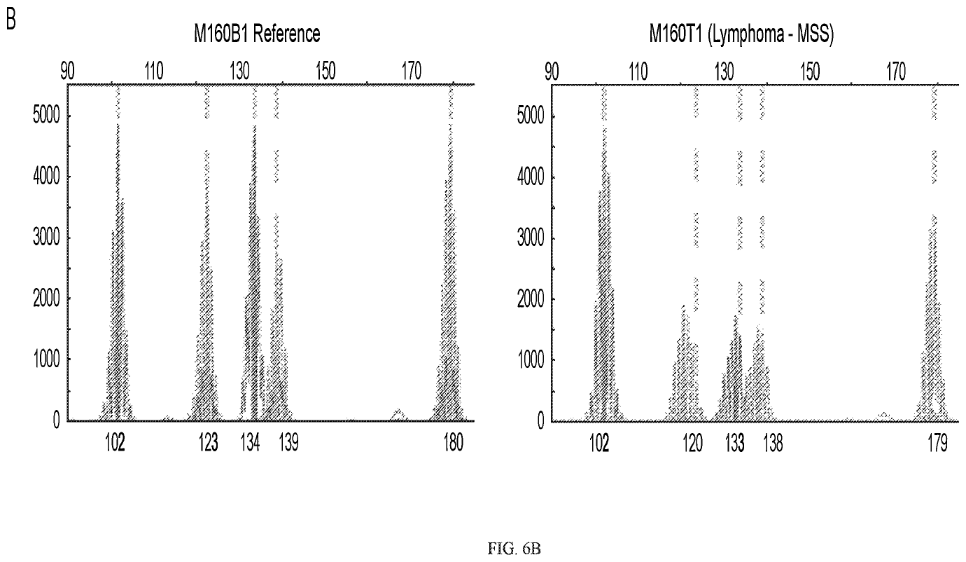

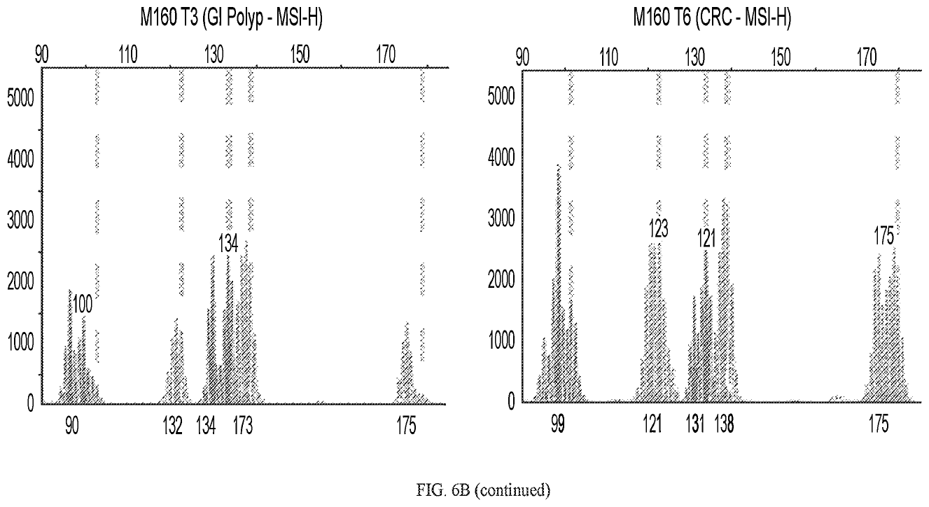

Since cells from patients with CMMRD have impaired abilities to repair mismatches and MS-indels, MSI should be prevalent in all normal and cancerous tissues. To test this hypothesis, the MSI status of a cohort of 96 CMMRD cancers and 8 normal tissues was analyzed using the Microsatellite Instability Analysis System (MIAS, Promega). Strikingly, only 17 (18%) of the 96 CMMRD cancers and none of the normal tissues were classified as MSI-H (≥2 loci with ≥3 bp indels) (FIG. 1A). This observation was not related to the specific MMR mutated gene (P=0.95, Kruskal-Wallis, FIG. 6A) and similarly, different tumors from the same patient resulted in different MSI classifications (FIGS. 1A and 6B, Table 1). Interestingly, observed tissue specificity was observed with gastrointestinal (GI) cancers having the highest proportion of MSI-H (10/25, 40%) compared to brain tumors (2/51, 4%, P=2×10-4, Chi-squared test).

It was unclear whether the lack of indels in the five MIAS microsatellite loci (MS-loci) was a unique feature to these loci, or whether MS-loci in CMMRD tumors universally have a low rate of indel accumulation. To test this, MSMuTect was applied to all the MS-loci in the 69 tumor/normal whole-exome pairs of pediatric CMMRD cancers. It was found that pediatric CMMRD cancers had a significantly higher tumor MS-indel burden (TMSIB) than pediatric MMR-proficient (MMRP, n=239) cancers (P<2.0×10-15, Mann-Whitney U-test, FIG. 1B). Importantly, pediatric CMMRD cases had a similar TMSIB to the adult MMRD cases (n=114, P=0.48), and no significant difference was found in the TMSIB across pediatric cases from different tissues (FIG. 1B). On the other hand, the number of SNVs in the pediatric tumors were higher due to the somatic POLE mutations commonly found in CMMRD tumors. Overall, the method demonstrates that, as predicted by the lack of MMR, pediatric CMMRD cancers indeed have an increased rate of MS-indels accumulation, similar to adult MMRD cancers.

MS-indels were spread equally on different chromosomes in both adult and pediatric cohorts (FIG. 1C, Table 2), and increased between initial and relapsed tumors (n=8) (P=0.014, Paired-samples Wilcoxon Test, FIG. 1D). Furthermore, in contrast to adult MMRD tumors which exhibit highly mutated loci (hotspots) like those used in the MIAS assay, pediatric MMRD tumors lacked this characteristic. For example, the microsatellite in the ACVR2A gene (chr2:148683686-148683693) is mutated in ˜45% of adult MMRD cases, but only 11% in pediatric CMMRD cases. In addition, while adult tumors had 19 hotspots that were mutated in more than 30% of the cases, the strongest hotspot in pediatric tumors was mutated in ˜20% (FIG. 1E, Table 3). Together, these observations indicated that MS-indels accumulate in childhood CMMRD cancers, and continue to accumulate as tumors progress. Furthermore, the key differences between adult and childhood MMRD cancers, i.e. the different mutated loci and the lack of MS-loci that were mutated very frequently, may explain why the MIAS assay was unable to detect MSI in pediatric tumors.

### Example 2: MMRD and Polymerase Mutant Tumors have Distinct Microsatellite Indel Signatures

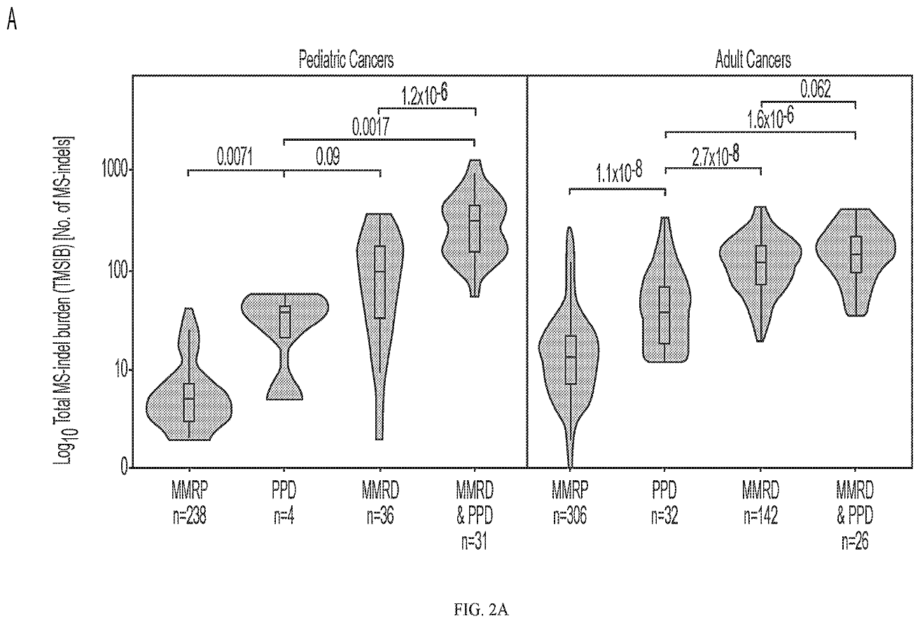

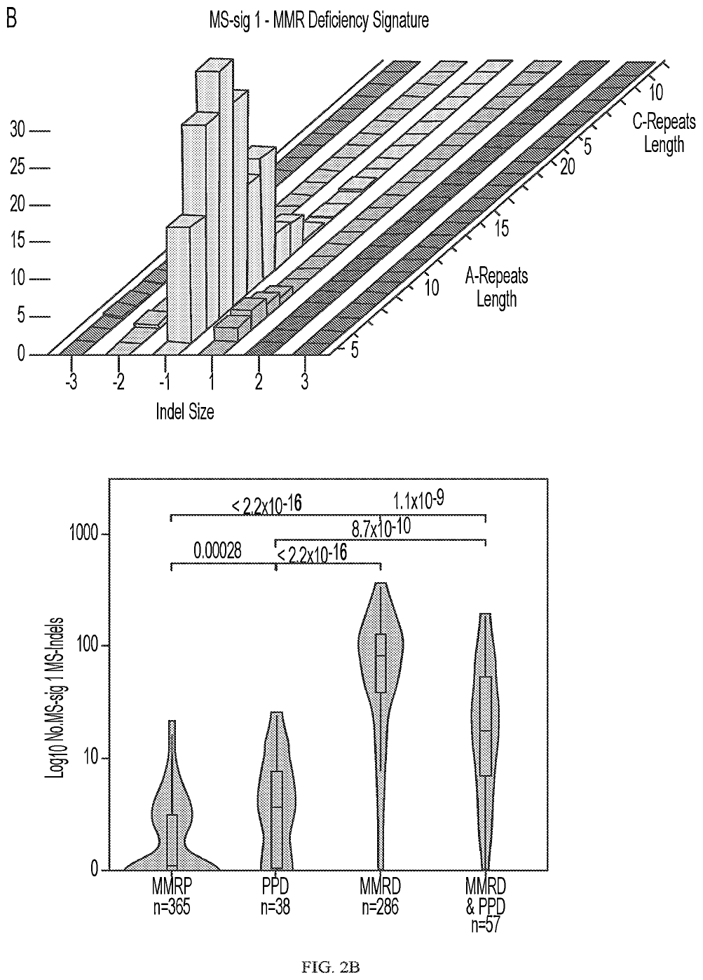

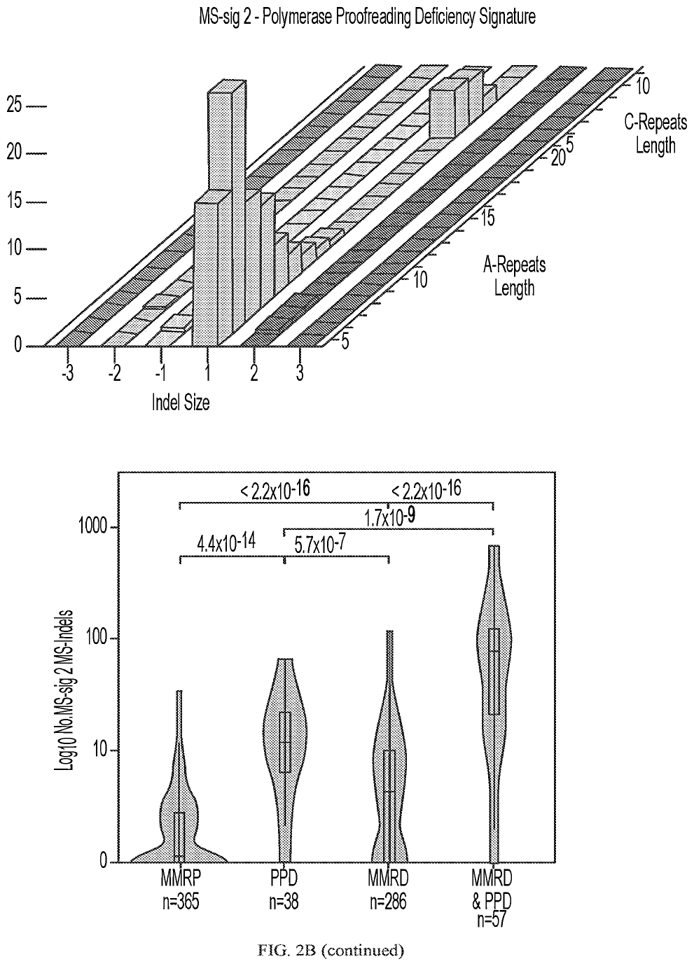

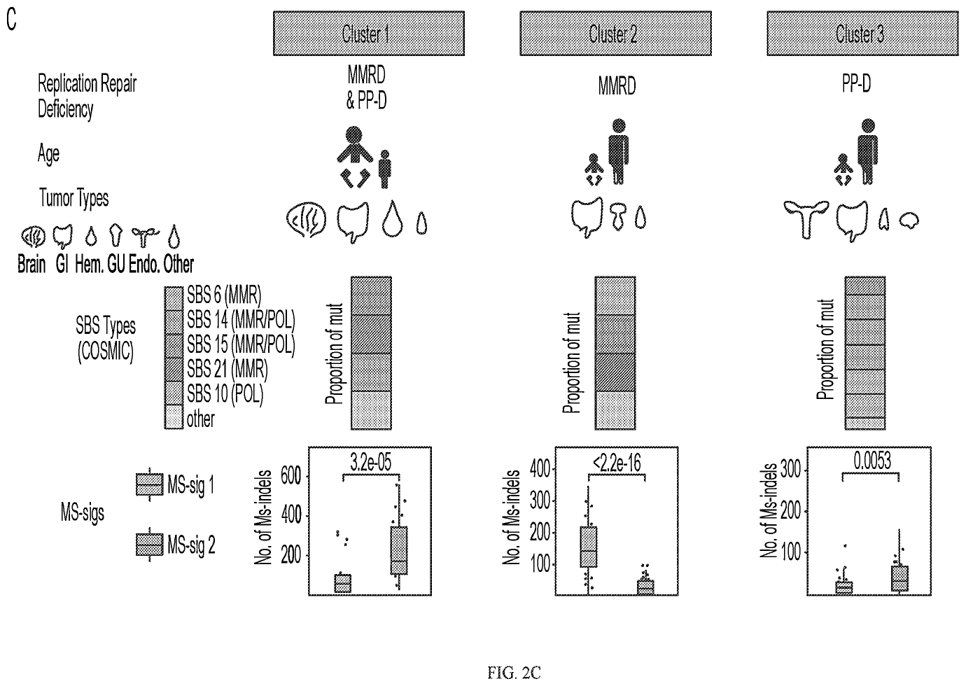

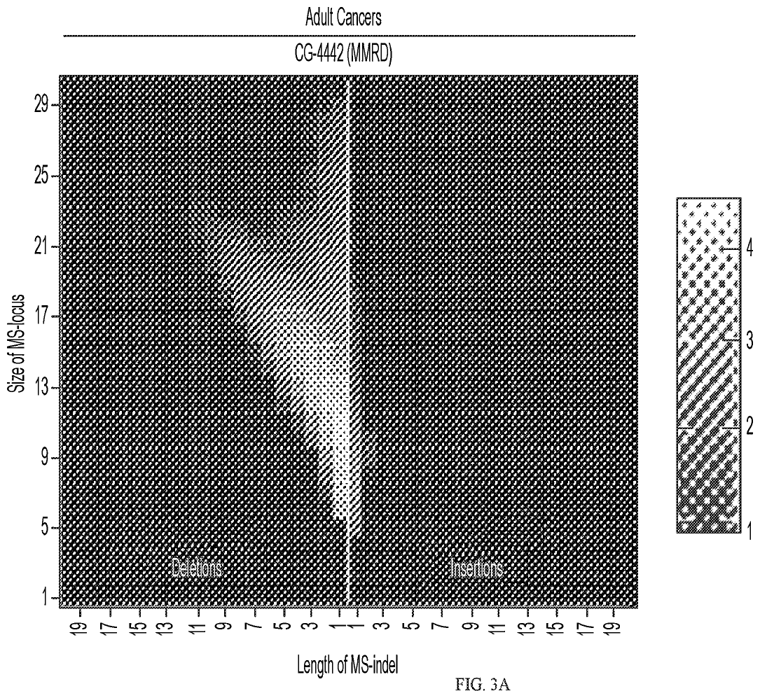

Another striking observation was that childhood tumors had a similar number of MS-indels as adult MMRD cancers, despite being largely microsatellite stable in the MIAS analysis (FIG. 1A). This was most clearly observed between childhood CMMRD brain tumors and adult GI cancers (P=0.27, Mann-Whitney U-test, FIG. 1B), as well as between pediatric and adult GI cancers (P=0.37, Mann-Whitney U-test, FIG. 1B). Since CMMRD tumors acquire somatic polymerase mutations, which are known to dramatically increase SNV mutations in cancer and MS-indels in yeast, it was hypothesized that mutant polymerases actively contribute to MS-indel accumulation in pediatric CMMRD tumors. Therefore MS-indels found in whole exome sequencing of 239 pediatric MMRP, 38 MMRD, 4 polymerase mutant and 31 combined replication repair deficient (MMRD& polymerase mutant) tumors were compared to 505 adult endometrial cancers spanning the same four subtypes. Despite proficiency in MMR, polymerase mutant cancers exhibited increased MS-indel burden in both pediatric and adult cancers (P=0.0071, P=1.1×10-8, respectively; FIG. 2A). Moreover, combined replication repair deficient cancers have an increased MS-indel burden compared to MMRD cancers in the pediatric cohort (P=1.2×10-6, FIG. 2A, left panel), further highlighting the contribution of polymerase mutations to the accumulation of MS-indels.

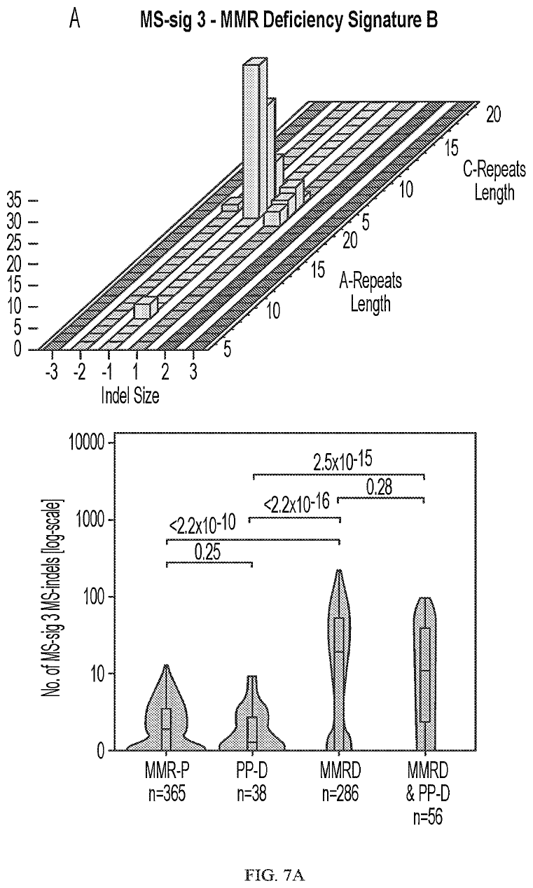

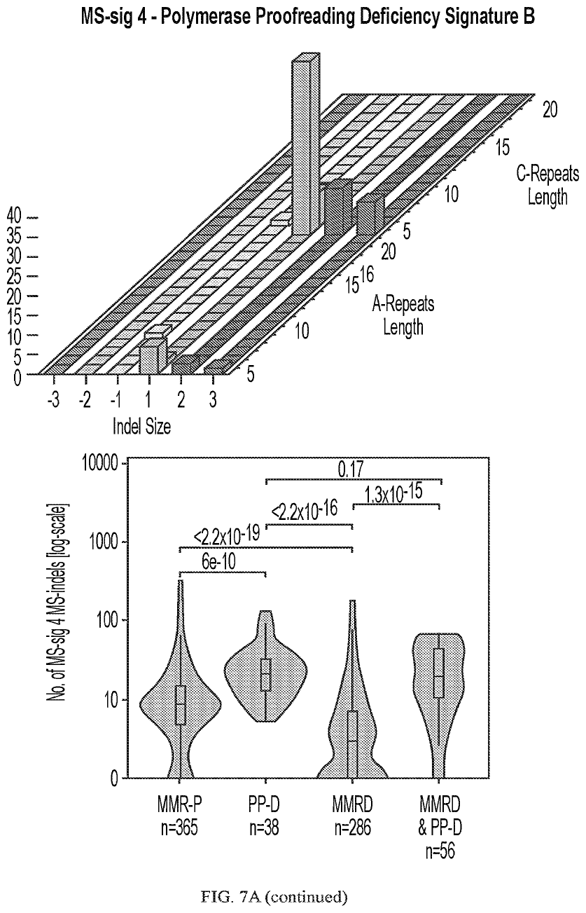

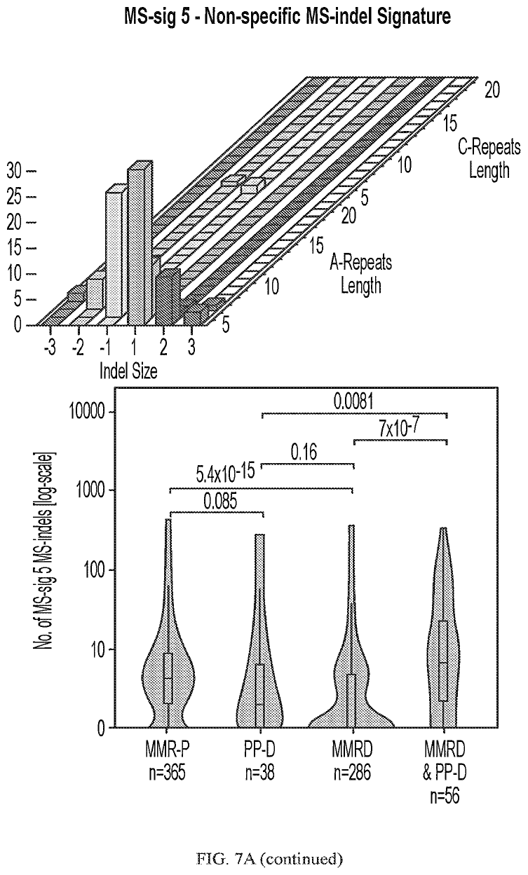

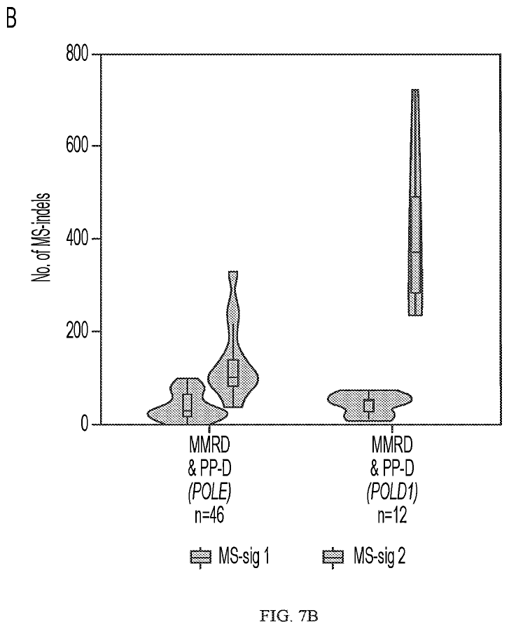

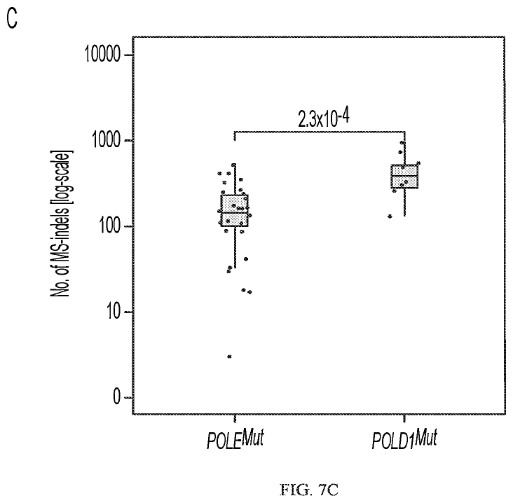

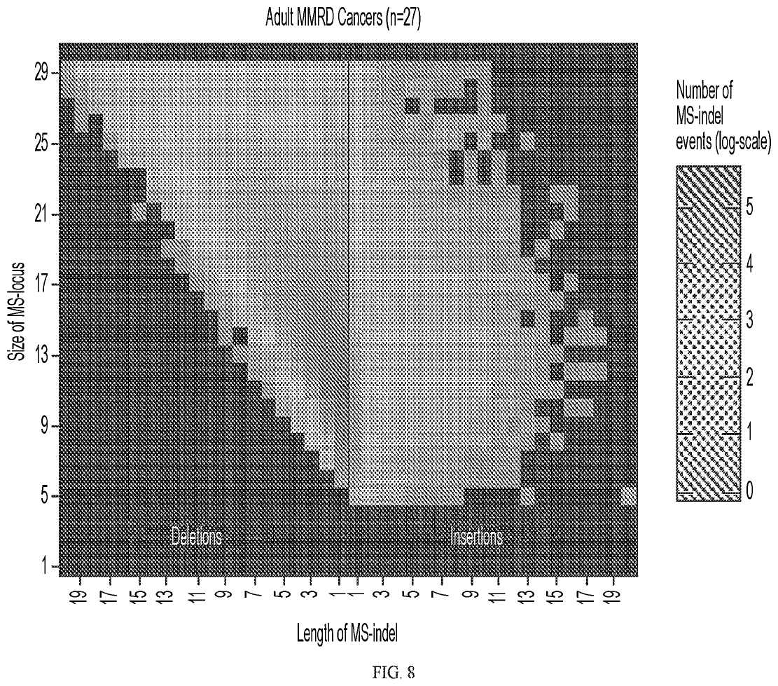

It was then wondered whether the two components of the replication repair machinery exert distinct alterations in microsatellites. To investigate this, each MS-indel based on three features, its motif (A or C), the microsatellite length (5-20 bp) and the indel size (−3-+3 bp) were characterized. SignatureAnalyzer was then used to infer the different mutational signatures based on these 192 (=2×16×6) configurations, and found five distinct MS-indel signatures (MS-sigs; FIGS. 2B and 7A). When compared to MMR proficient cancers (FIG. 2B), MMRD cancers were enriched for two signatures dominated by 1 bp deletions in both A- and C-repeats (MS-sigs 1 and 3, Mann-Whitney U-test P<2.2×10-16), whereas polymerase mutations resulted in two signatures dominated by 1bp insertions (MS-sigs 2 and 4, P=4.4×10-14). Additionally, replication repair deficient cancers with driver mutations in POLD1 (encoding the DNA polymerase that synthesizes the lagging strand) were analyzed (FIGS. 7B, and 7C). POLD1mut cancers were similarly dominated by insertions in MS-indels as POLEmut cancers (FIG. 7B). This is the first time that the unique mechanism of MS-indel accumulation by DNA polymerase was described in human cancers. These results contrast with other yeast reports, which suggest that mutant POLD1 creates single base deletions in microsatellite sequences. Together, these findings support that POLE and POLD1 with defective proofreading are associated with increased insertion events (as opposed to the deletion events associated with MMRD) and can contribute to the overall total microsatellite indel (MS-indel) burden in human cancers.

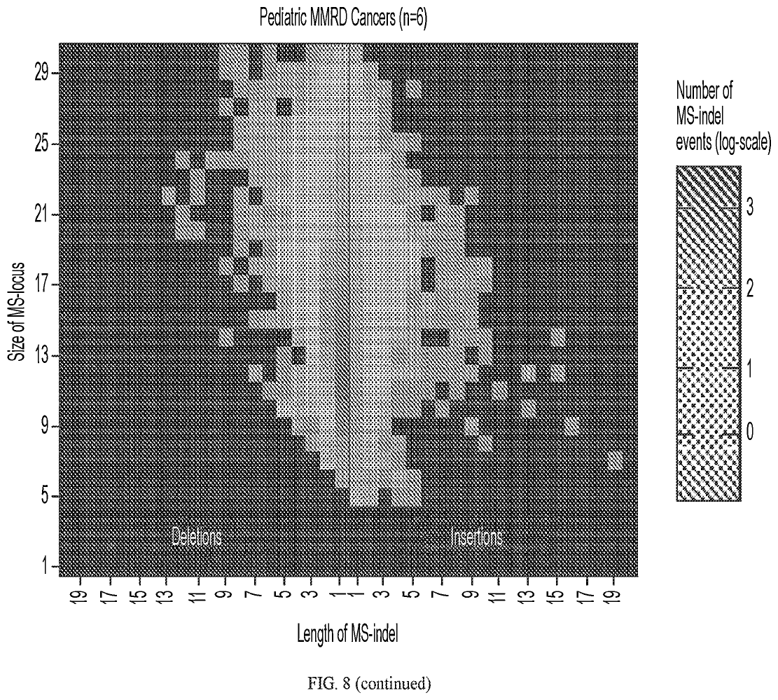

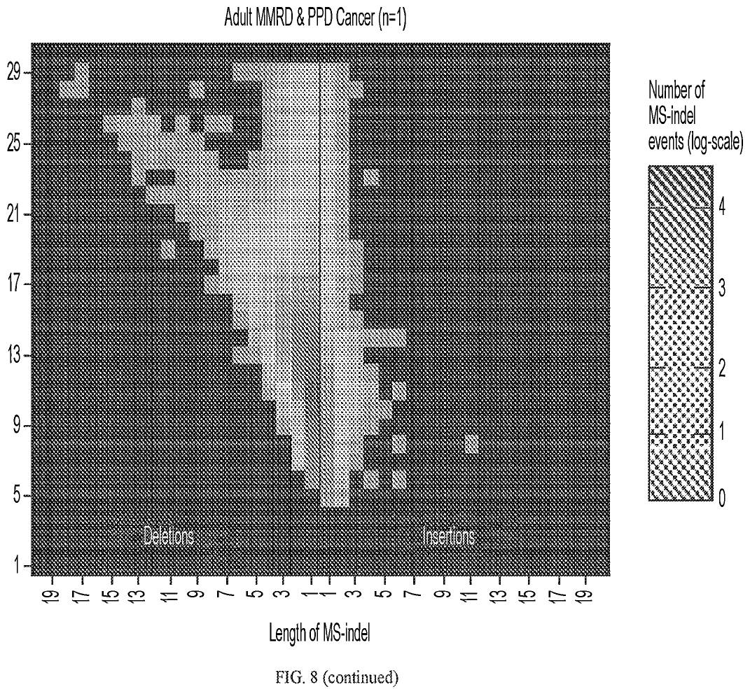

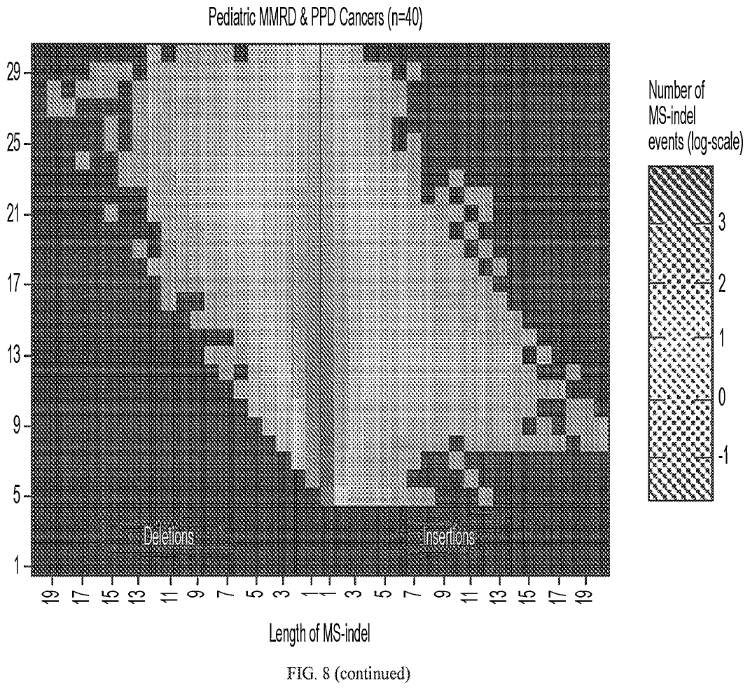

Previously, hypermutant cancers were classified into three clinically relevant subtypes of replication repair deficient (MMRD, PPD, or both MMRD & PPD) based on their single-base substitution signatures (COSMIC SBS signatures, FIG. 8). Therefore, the correlation of the MS-sigs to these clusters was examined. Not intending to be bound by theory, MMRD&PPD (SBS-Cluster 1) are mostly seen in children with germline MMRD that later acquire somatic POLE mutations, and fit a combination of MS-sig1 and MS-sig2. MS-sig1 was specific for MMRD-only cancers (SBS-cluster 2) in both children and adults, and PPD (SBS-Cluster 3) cancers are highly driven by the mutant polymerase and have more MS-sig2 MS-indels than MS-sig1 (P=3.2×10-5, P=0.0053, and P<2.2×10-16 respectively, FIG. 2C). These data suggest that both SNV-based signatures and MS-sigs provide additional complementary information to standard mutational calls, and can be used to clinically screen and stratify tumors in the future.

### Example 3: Accumulation of Large Microsatellite Deletions in MMRD Cancers with Preferred Final Length

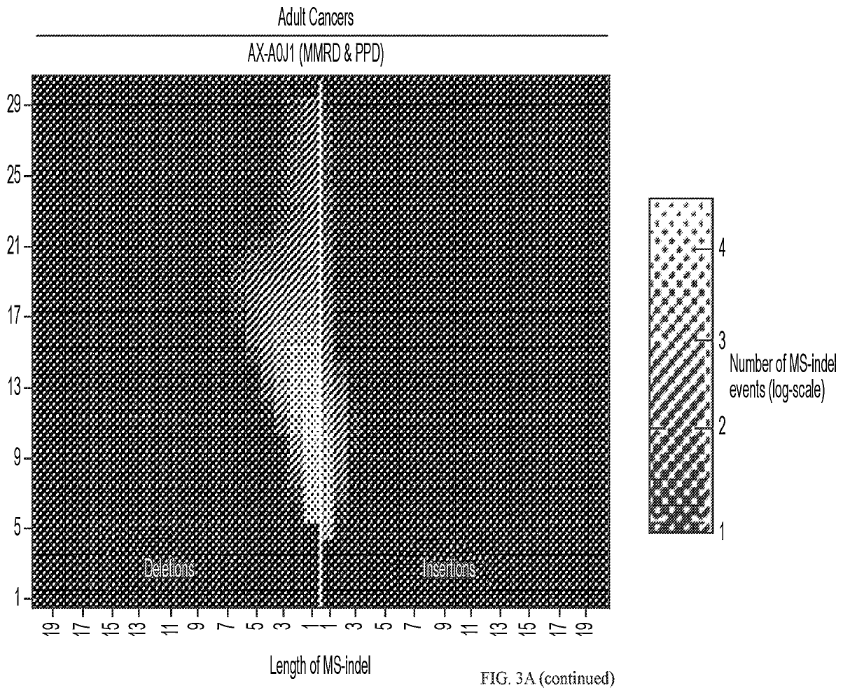

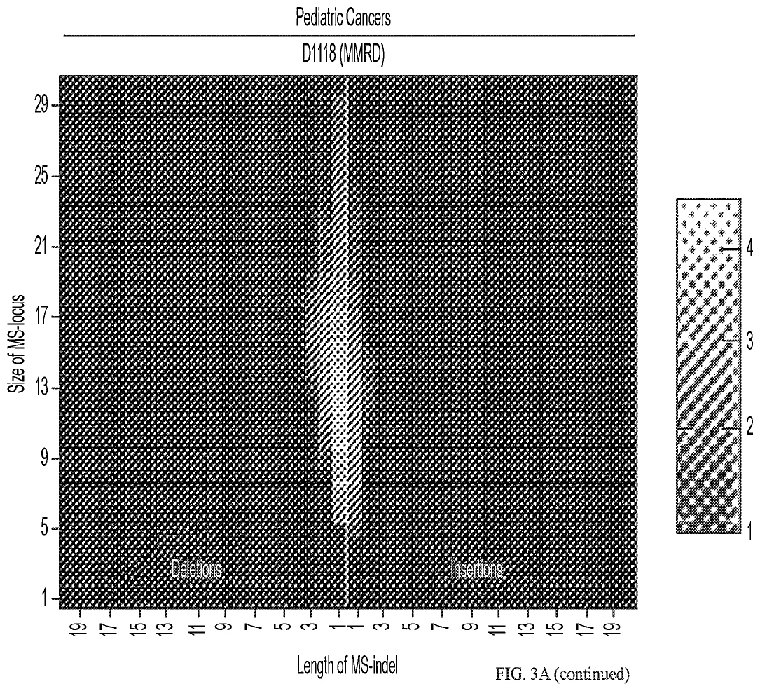

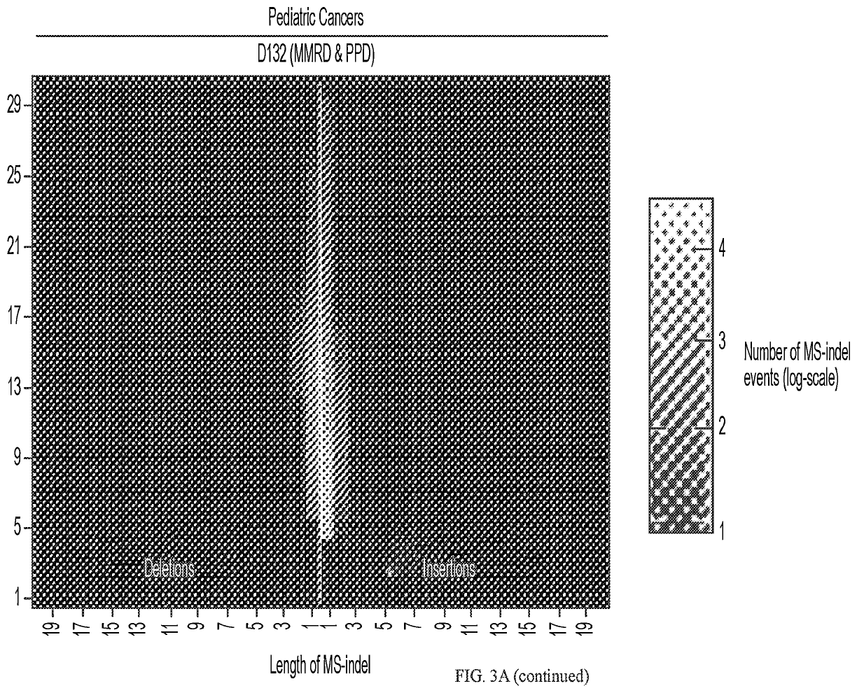

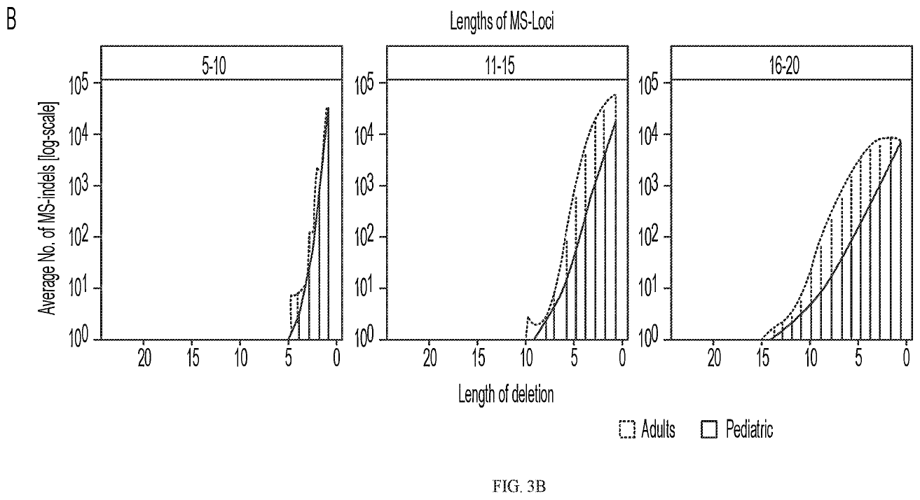

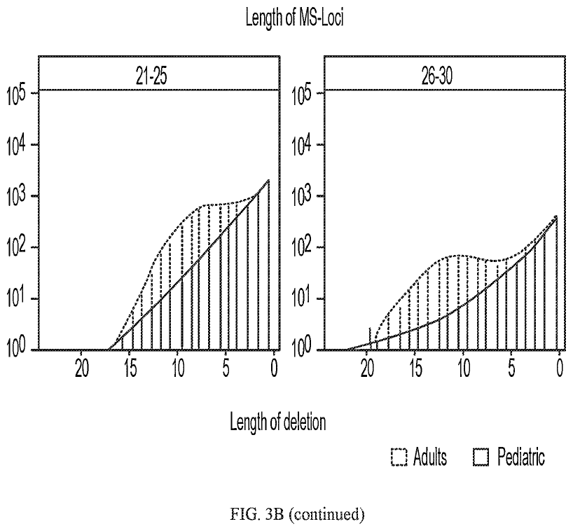

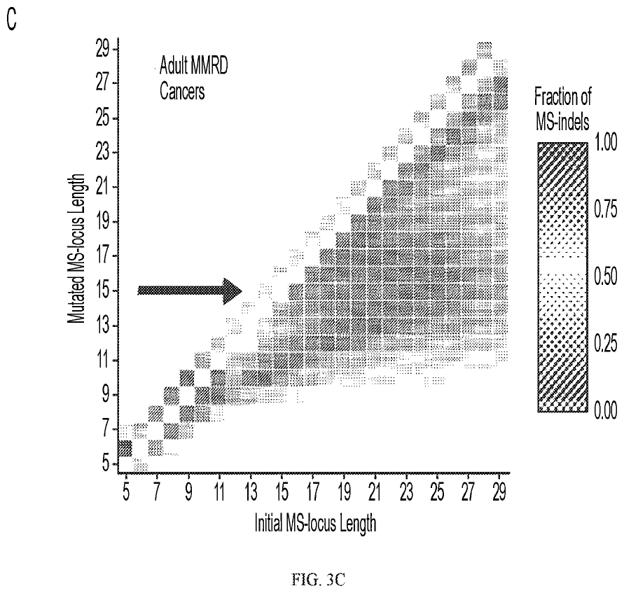

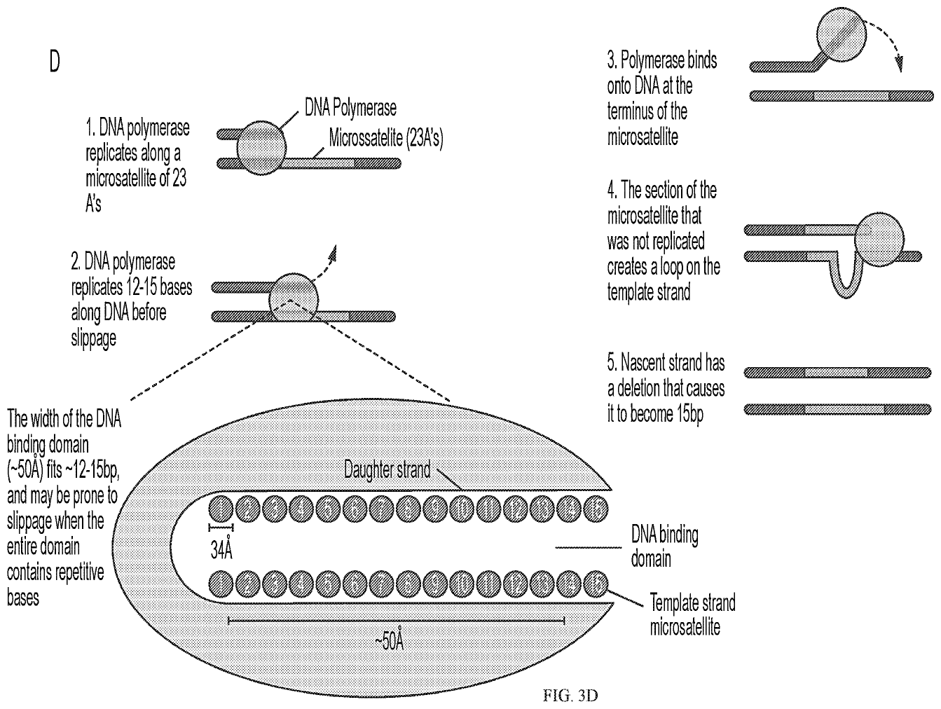

To gain insight into the mechanism of MS-indels accumulation during replication repair deficient tumorigenesis, the patterns of MS-indels in whole genomes of 46 pediatric replication repair deficient cancers (39 brain, 4 colorectal, 1 osteochondroma, 1 Wilm's tumor, and 1 T-cell lymphoma) and 28 MMRD adult cancers (12 colorectal, 5 gastric, 11 endometrial) were analyzed. The genome provided −50 times more MS-loci compared to the exome, and has disproportionally higher numbers of longer (>20 bp) MS-loci. These two features enabled a more refined analysis of the MS-indel distribution. Adult MMRD cancers present (FIG. 3A and FIGS. 8, 9A, 9B, Table 4) bias towards deletions similar to their behavior in whole exome sequencing (FIG. 2B). They also presented a new phenomenon of accumulating long deletions (>3 bases), which increased in size with increasing MS-loci length. In contrast, pediatric CMMRD cases have mostly 1bp deletions and MMRD&PPD cancers were enriched with 1bp insertions (FIGS. 3A, 8, 9A, and 9B, and Table 4) with a lack of long deletions. Not intending to be bound by theory, the lack of long MS-indels in pediatric cancers was likely the reason for the inability of the conventional electrophoresis methodologies (e.g. MIAS, Promega, FIG. 1A) to detect MSI in these cancers, as the standard assay cannot robustly identify short indels and only considers indels of bases as robust events.

To better understand the kinetics of the accumulation of large deletions in MS-loci, two models were compared. (i) A two-phase model where each mutational event can alter either a single repeat motif or multiple motifs; and a simple (ii) stepwise-mutation model, which assumes only one repeat motif is altered per mutational event, making large deletions the amalgamation of multiple deletions. The stepwise model predicted that the relationship between the MS-indel size and the frequency of the mutational events of that size would be unimodal (i.e. Poisson). On the other hand, the two-phase model enabled a bimodal relationship between the MS-indel size and frequency.

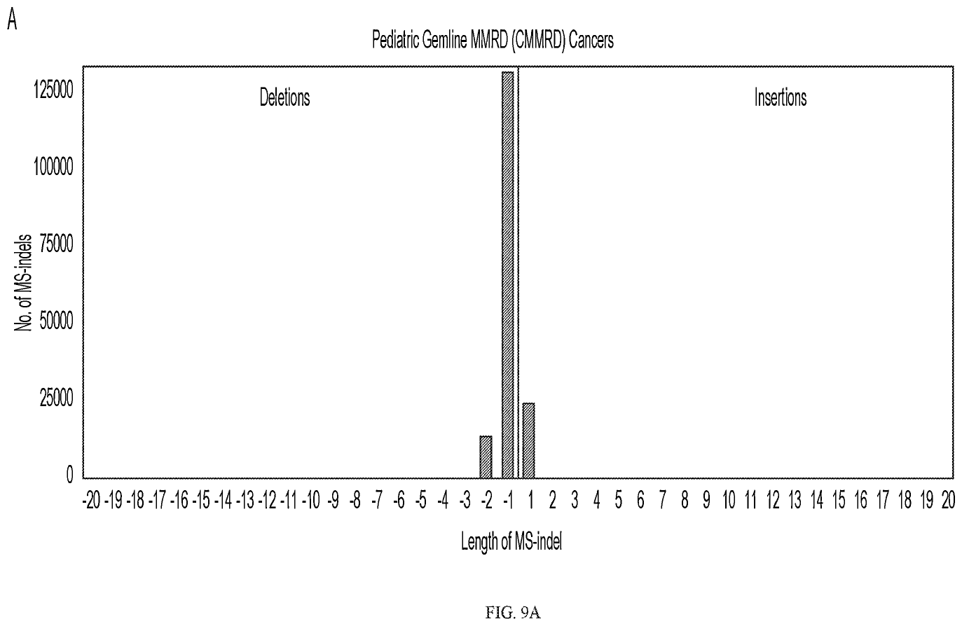

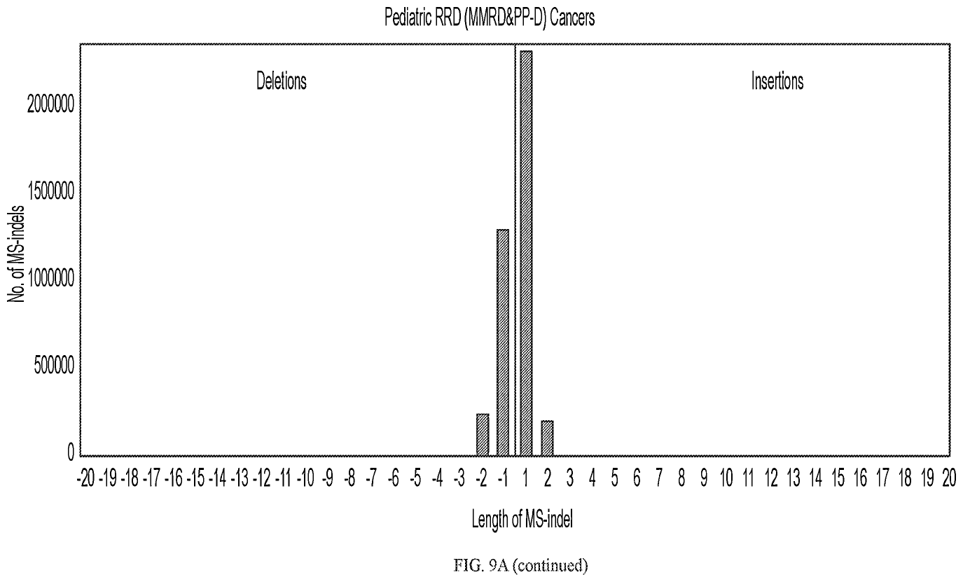

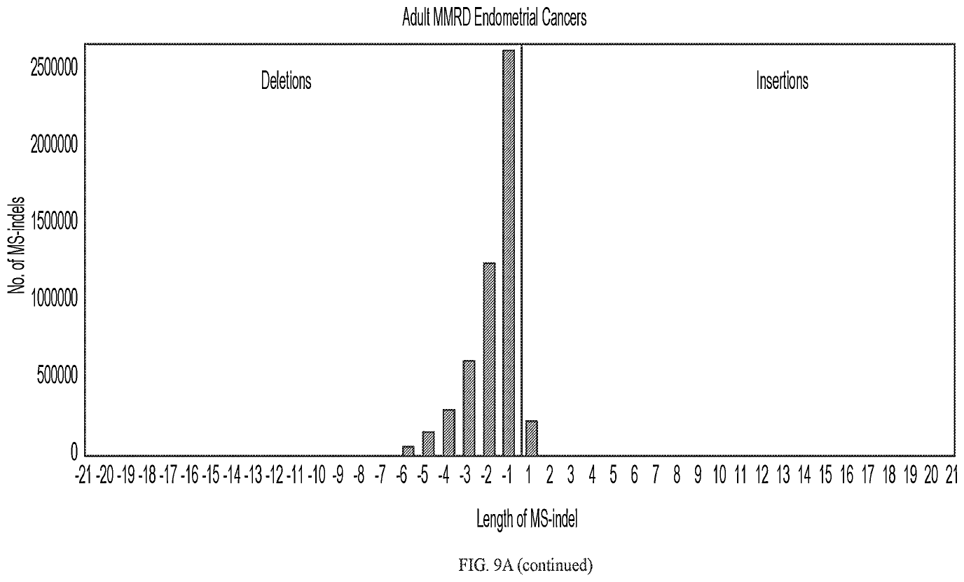

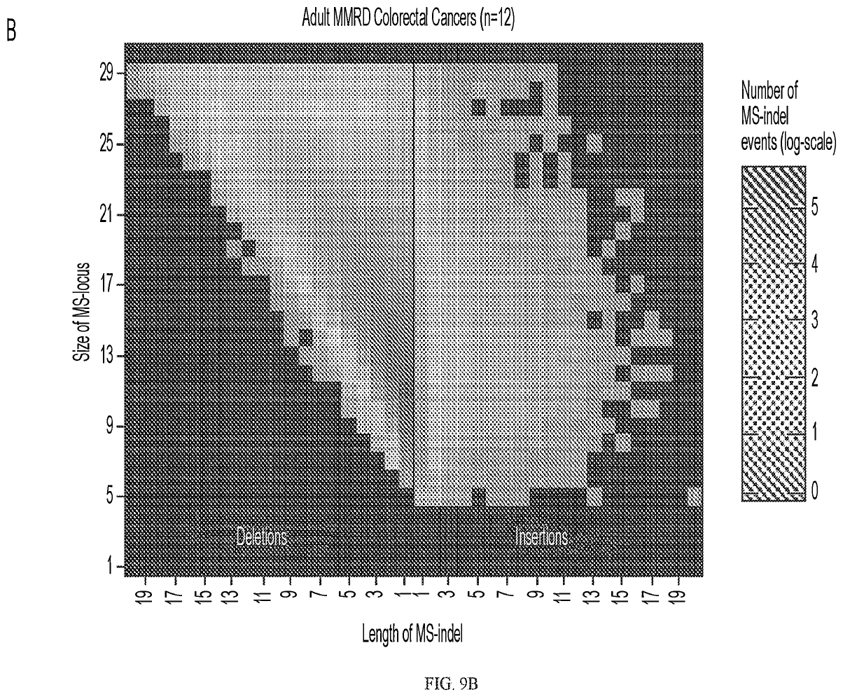

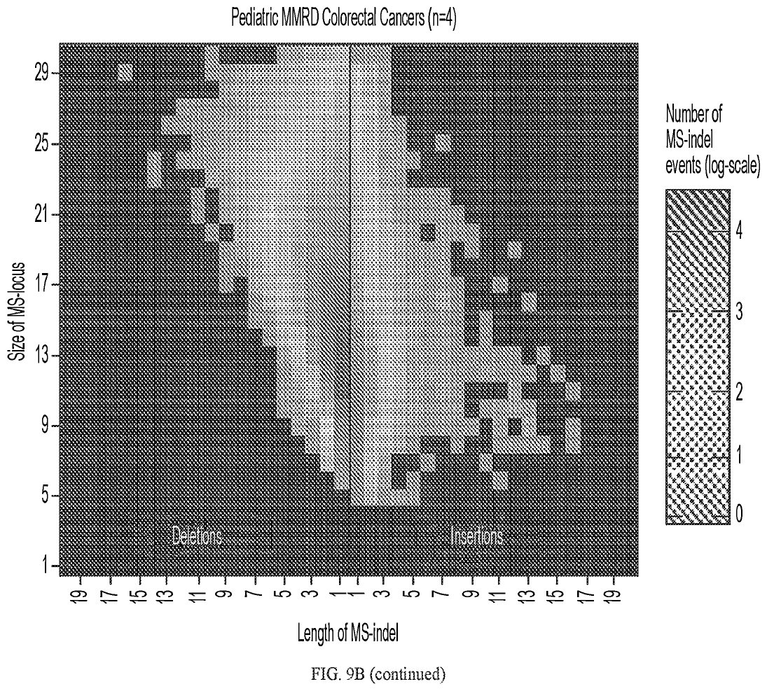

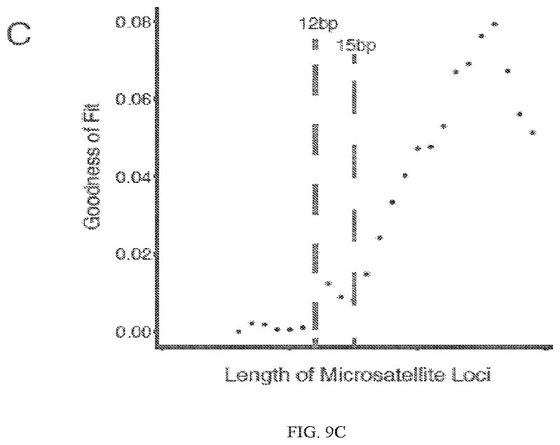

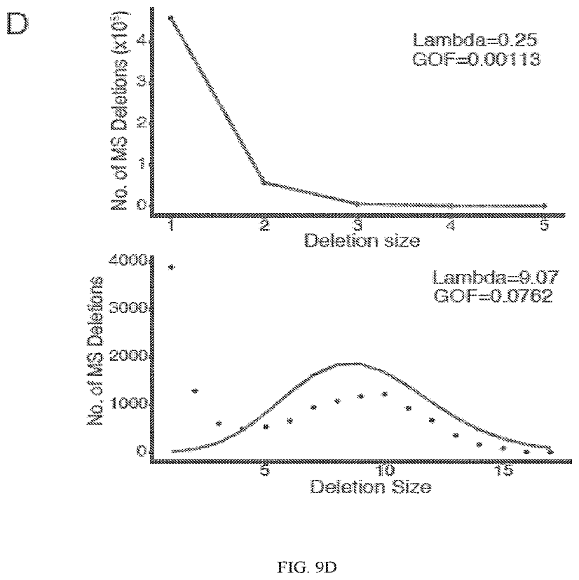

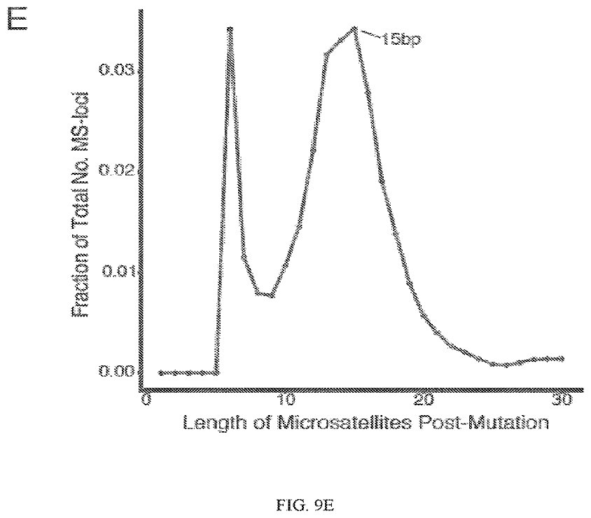

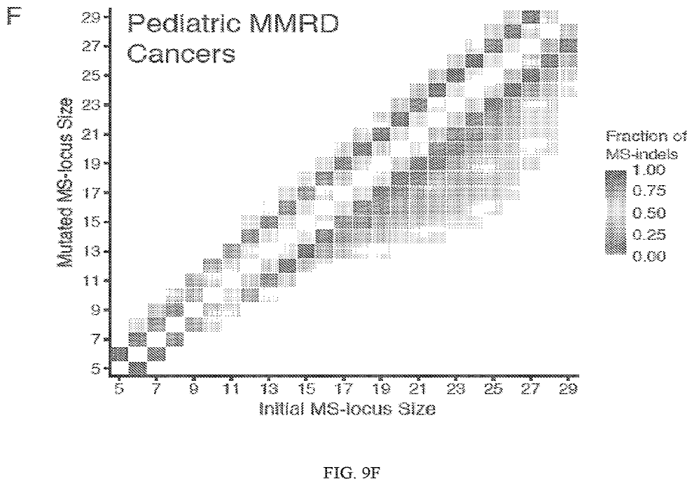

The pediatric tumors followed the predictions of the stepwise model and presented a sharp decrease in the frequency of MS-indels as the deletion size increases (FIG. 9F). In contrast, the adult tumors followed the stepwise model only for short loci (<12 bp), and from 12 bp and onwards, they deviated from the stepwise model and resembled the two-phase model (FIGS. 3B, 9C, and 9D).

Interestingly, the large deletions in adult tumors reduced the length of long (>15 bp) MS-loci to become −15 bp (FIGS. 3C and 9E). These findings suggested that there was an underlying mechanism of MS deletions that gave larger loci the tendency to converge to 15 bp in length. Not intending to be bound by theory, a potential model for this phenomenon was that after the DNA polymerase replicated 12-15 bp on a microsatellite locus, it may become more susceptible to slip off from the template strand. The width of the DNA-binding domain of the polymerase is known to be ˜50 Å, which is approximately the combined length of 15 nucleotides (51 Å) in a DNA double helix. It was predicted that because 15 nucleotides span the entire DNA-binding domain, the weak association of the polymerase to repeated nucleotides makes the polymerase especially prone to detach from the template strand. As the new strand then rebinds to the template strand, the template strand may generate a loop to become fully complementary to the ˜15 bp of the nascent strand (FIG. 3D). This process is restricted to deletions, hinting that it may be specific to MMR deficient cells, whereas insertions in polymerase mutant cancers did not reveal the same large events (FIG. 9A).

### Example 4: Clinical Applications of MS-Sigs

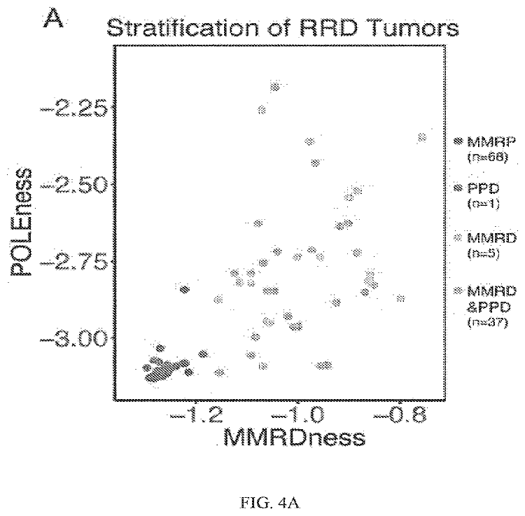

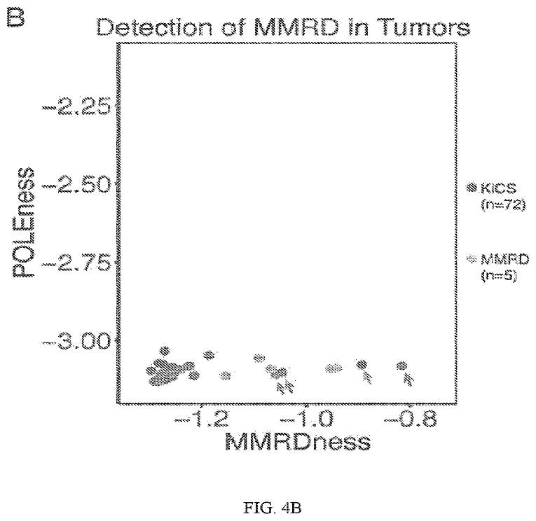

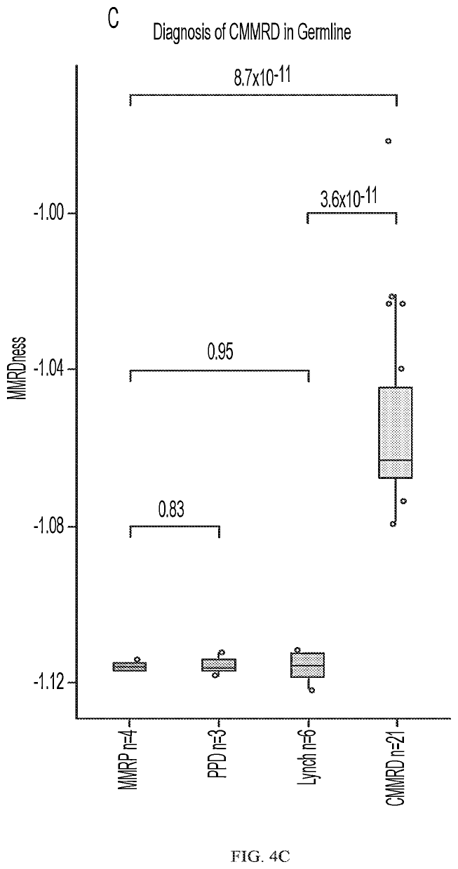

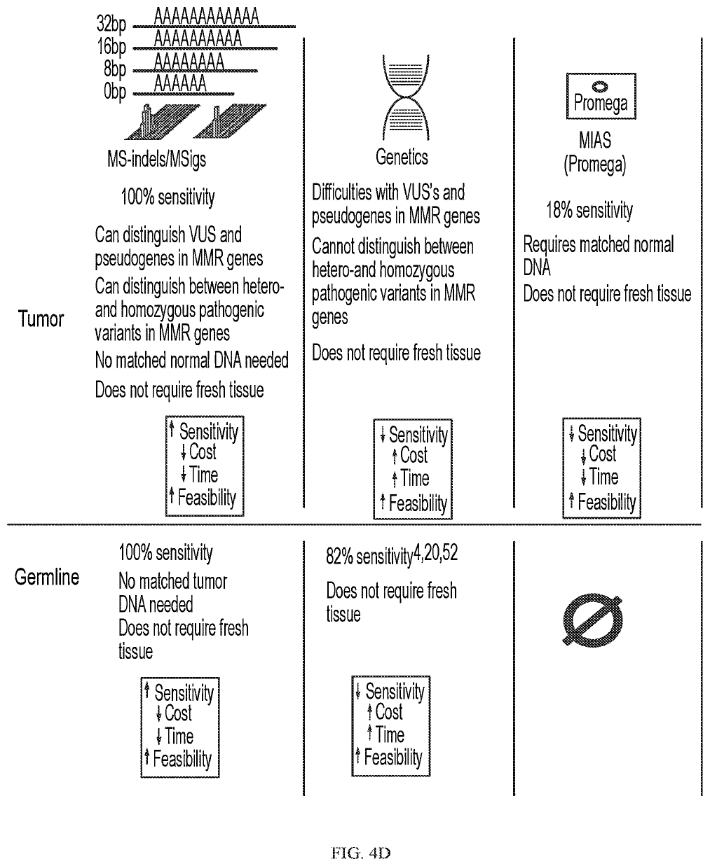

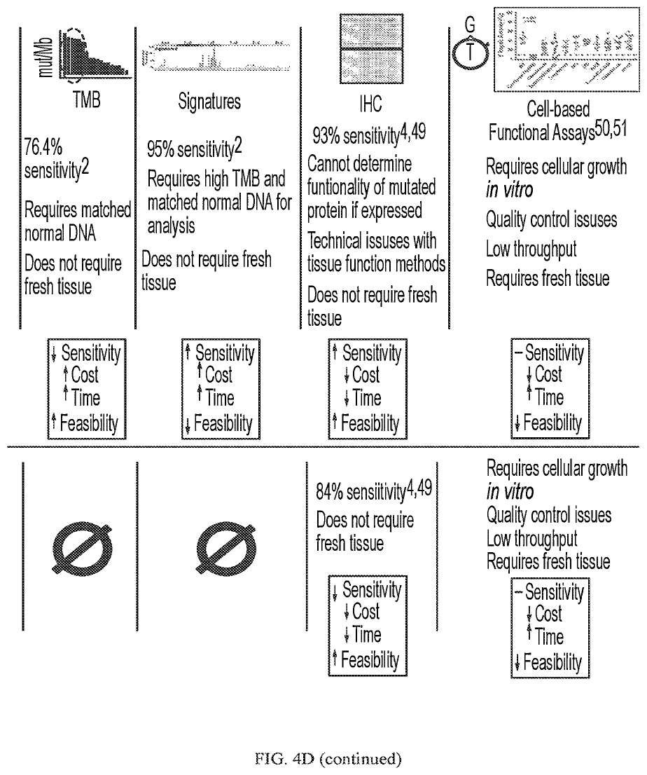

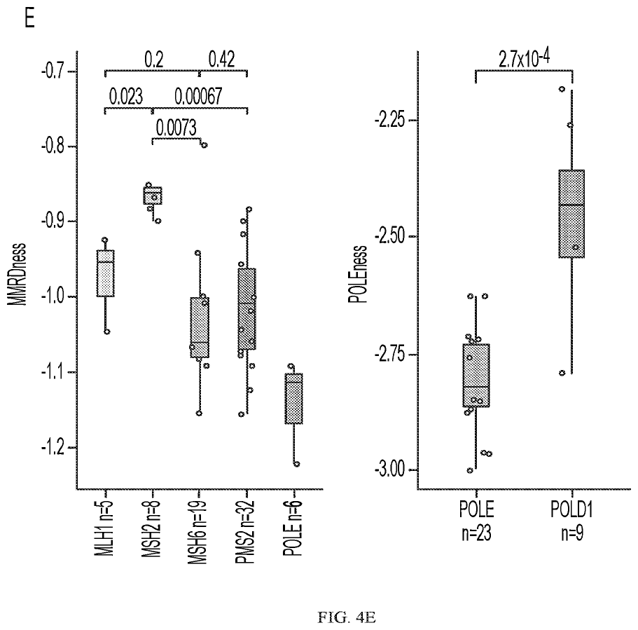

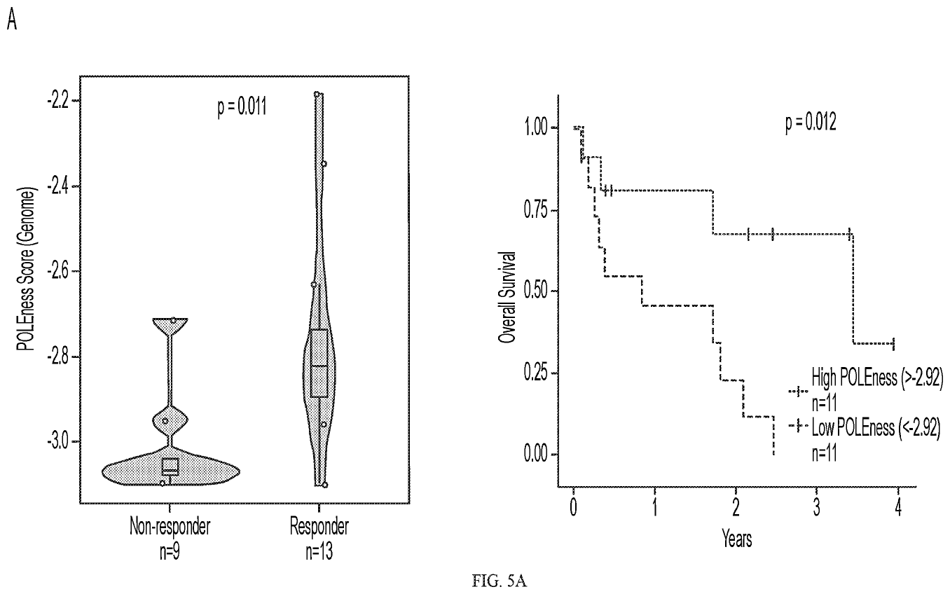

To begin answering the potential diagnostic and predictive role of MS-indels in patients with replication repair deficient cancers, the ability of MS-sigs to diagnose replication repair deficient cancers was first compared with currently used SNV-based clinically approved tools. The MS-indel signatures presented above (MS-sig1 and MS-sig2) were based on detecting MS-indels by comparing pairs of tumor and normal samples. However, the characteristic patterns of MS-indels and the large number of microsatellites in the genome could enable analyzing tumors without the need for matched normal samples. To test this, scores were designed that reflect the prevalence of MS-sig1 (MMRDness score) and MS-sig2 (POLEness score) in any given sample. Both scores were calculated by taking the logarithm (base 10) of the ratio of the number of −1 deletions (MMRDness) or +1 insertions (POLEness) divided by the total number of MS-loci within the specified lengths (10-15 bp for MMRDness and 5-6 bp for POLEness). Higher scores indicate higher prevalence of the MMRDness and POLEness signatures in each sample. Only MS-sig1 and MS-sig2 were used due to the overwhelming dominance of A-repeat MS-loci in the genome compared to C-repeats, which were represented by MS-sigs 3 and 4. The two scores were calculated for a well-characterized set of tumors and were able to separate the four subtypes analyzed (MMRP, MMRD, PPD and MMRD&PPD) (FIG. 4A).

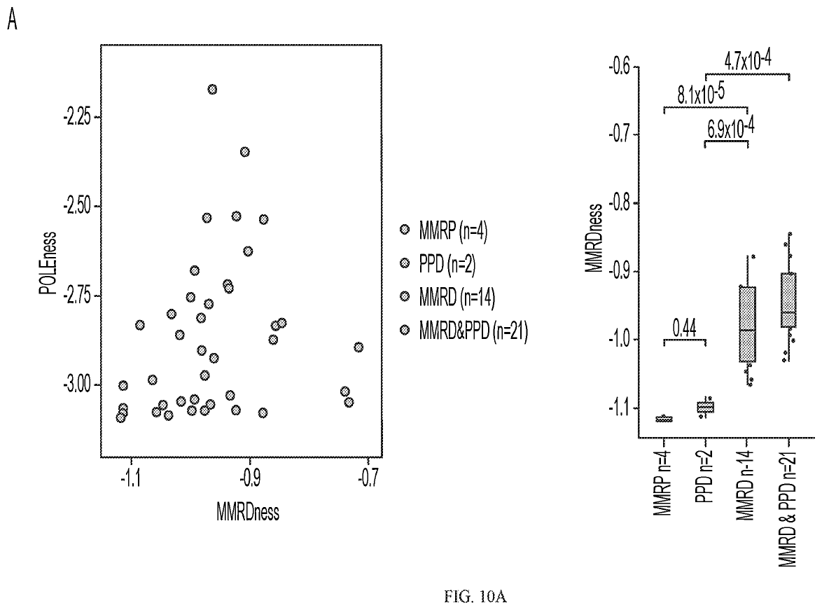

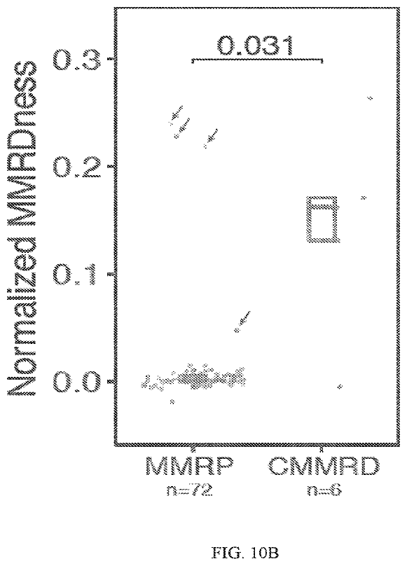

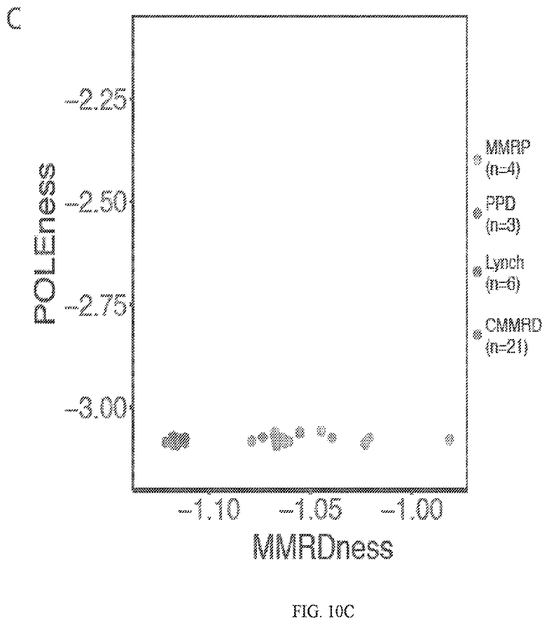

It was then validated whether the MMRDness and POLEness scores can be used as a more cost-effective clinical tool for detecting MMRD and PPD. 52 MMRP and replication repair deficient tumors were sequenced with a small fraction of the standard genomic coverage (0.5× instead of the typical 30×), and calculated their MMRDness and POLEness. Similar clustering of the replication repair deficient subtypes was observed (FIG. 10A), validating the ability of the two scores to accurately and cost-effectively classify replication repair deficient tumors. Subsequently, it was tested whether the MS-sigs can improve the current SNV-based methods used to screen and diagnose replication repair deficient tumors. MMRDness and POLEness scores of 72 tumors for which WGS was available were assessed through the clinical KiCS sequencing program. The MMRDness score uncovered four tumors (FIGS. 4B and 10B) that were not known to be MMRD by the sequencing center, as they had relatively low tumor mutational burdens and no SNV-based MMRD signatures (3-17 Mut/Mb, Table 5). Genetic testing revealed that three of them had canonical Lynch syndrome germline mutations and the remaining one carries a novel germline MMR mutation (Table 5). This suggested that MMRDness and POLEness was both a more accurate and less expensive test compared to current methods for detecting replication repair deficient in tumors.

### Example 5: Analysis of Whole Genome Microsatellite Instability Signatures can Diagnose CMMRD in Normal Cells

Having established the ability of MS-sigs and the corresponding MMRDness score to identify MMRD tumors, the ability of the tool to identify germline (CMMRD) mutations in non-malignant (blood) samples was tested. All non-malignant samples from CMMRD patients (n=34) had an elevated MMRDness score compared to MMR proficient, polymerase mutant, and Lynch syndrome patients (P=8.7×10-11, Mann-Whitney U-test, FIGS. 4C and 10C). Notably, the Promega panel assay was completely unable to detect CMMRD in non-malignant tissues (FIG. 1A) and, similarly, SNV-based signatures were not able to detect hypermutation in normal cells. The MMRDness score neither required a patient-matched normal control nor high sequencing depth to detect the MS-sig patterns throughout the genome that correspond to CMMRD. The high specificity and sensitivity of the MMRDness score (FIG. 4D) enables it to be an inexpensive and robust assay for screening and diagnosing CMMRD prior to cancer, which can be used to implement early surveillance protocols to improve the survival of CMMRD patients. On the other hand, the diagnostic role of POLEness was limited to replication repair deficient tumors (FIG. 4A).

### Example 6: Different Alterations of the MMR and Polymerase Genes have Varying Effects on MMRDness and POLEness

The four genes MLH1, MSH2, MSH6 and PMS2, that when mutated lead to MMRD, play different roles in the MMR mechanism. However, thus far, no footprint has been found in the mutational landscape for the four different genes. For example, germline mutations in MSH2 and MLH1 have higher penetrance than MSH6 and PMS2, and are found most commonly in adult Lynch Syndrome cases. In contrast, for individuals with CMMRD, the reverse is observed. PMS2 is the most commonly mutated gene, followed by MSH6, and biallelic MSH2 germline mutations are extremely rare. These cancers have similar tumor mutation burdens (TMB) irrespective of the mutated MMR gene; thus, the different penetrance cannot be simply explained by a higher mutation rate. Other functional assays have also failed to explain this genotype-phenotype relationship despite the different mechanistic roles of the MMR genes. Similarly, PPD is almost strictly observed with POLE but not POLD1 germline mutations—an observation that also cannot be explained by differences in TMB or other functional assays.

It was therefore evaluated whether the deficiency in different genes may have distinct effects on the MMRDness score (FIG. 4E). It was found that MMRD cancers with biallelic inactivating mutations in MSH2 had the strongest MMRDness score, followed by MLH1, and was much lower in PMS2 mutant cancers (P=6.7×10-4, Mann-Whitney U test, FIG. 4E). This fit well with the clinical observations of mismatch repair deficiency, as MSH2 and MLH1 mutations had higher cancer penetrance than PMS2 and MSH6. This also correlated with the known mechanism of mismatch repair, as the MSH2 protein initially recognizes mismatches during replication and is crucial for both the mutSα and mutSβ complexes, whereas MLH1 is an important factor in the mutL complex.

Next, it was investigated whether the two polymerase genes (POLE and POLD1), have a distinct effect on the POLEness score. POLD1mut cancers had significantly higher POLEness score than POLEmut cancers (P=2.7×10-4, Mann-Whitney U-test, FIG. 4E), supporting the previous finding from the exomes (P=2.3×10-4, FIG. 7C), where POLD1mut cancers had a higher TMSIB than POLEmut. Not intending to be bound by theory, this may be explained by the replication of the lagging strand inherently having more dissociation and slippage of the polymerase from the DNA.

### Example 7: POLEness Score Predicts Response to Immunotherapy in Replication Repair Deficient Cancers

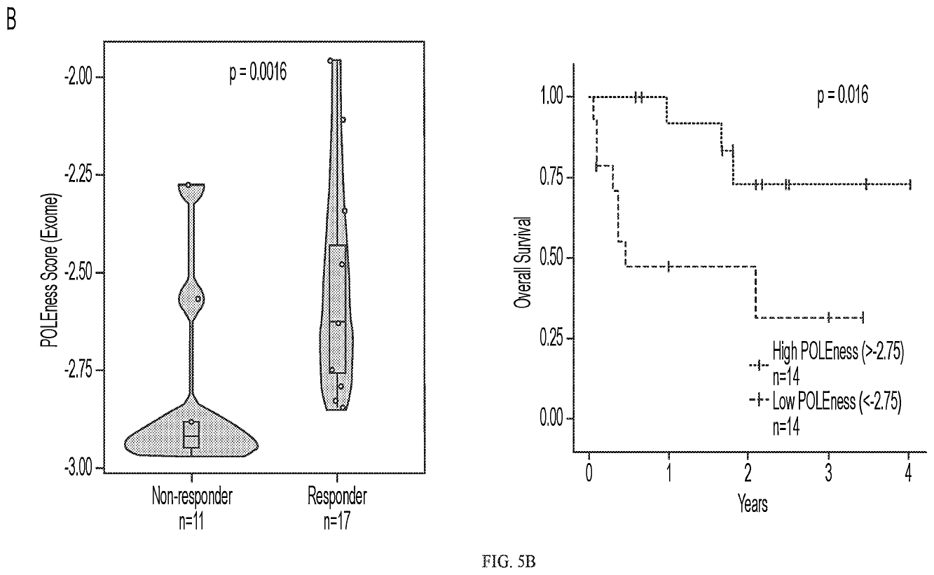

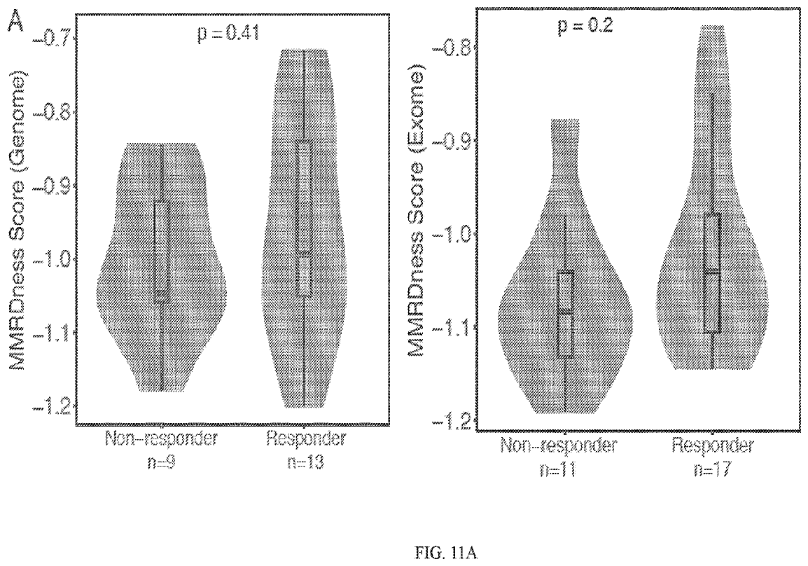

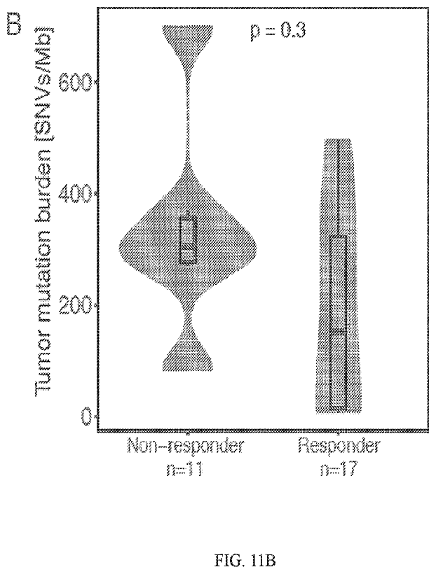

It was recently shown that replication repair deficient tumors tend to respond to immune checkpoint inhibitor therapy. However, not all patients respond, and currently there is no adequate method to distinguish responders from non-responders (FIG. 11B). It was hypothesized that within the context of replication repair deficient cancers, MMRDness and/or the POLEness score could predict response to immune checkpoint inhibitor therapy. To test this, WGS data of 22 replication repair deficient tumors undergoing immune checkpoint inhibitor as a part of a consortium registry study was analyzed. It was observed that the POLEness score of responders was higher than non-responders to immune checkpoint inhibitor (P=0.011, Mann-Whitney U-test, FIG. 5A) and tumors with a high POLEness score (>−2.92 POLEness) had a significantly higher survival than the low POLEness group (P=0.012, Log-Rank test, FIG. 5A). The ability of the POLEness score to predict response to immune checkpoint inhibitor was then tested using exomes from a larger cohort of patients (n=28). POLEness scores were higher in responders to immune checkpoint inhibitor therapy than in non-responders (P=0.0016, Mann-Whitney U-test, FIG. 5B, and Tables 6 and 7). However, exome and genome MMRDness was not shown to have a significant difference between responders and non-responders, potentially because all tumors in the cohort are MMRD (FIG. 11A, and Tables 6 and 7). The high-POLEness groups in both genome and exome had significantly longer survival times than the low-POLEness groups (P=0.012 and 0.016, Log-Rank Test, FIGS. 5A, 5B). This is the first time that a mutational signature was suggested as a biomarker to distinguish between responders and non-responders to immune checkpoint inhibitor in replication repair deficient tumors. This result shed new light on the immunogenicity of MS-indels and the use of their signatures as novel biomarkers for response to immune checkpoint inhibition in the context of replication repair deficient.

The above data demonstrates roles for both components of the replication repair machinery in MS-indel accumulation in cancers. These observations address important fundamental biological questions regarding accumulation of MS-indels during carcinogenesis, which can be translated to clinical use.

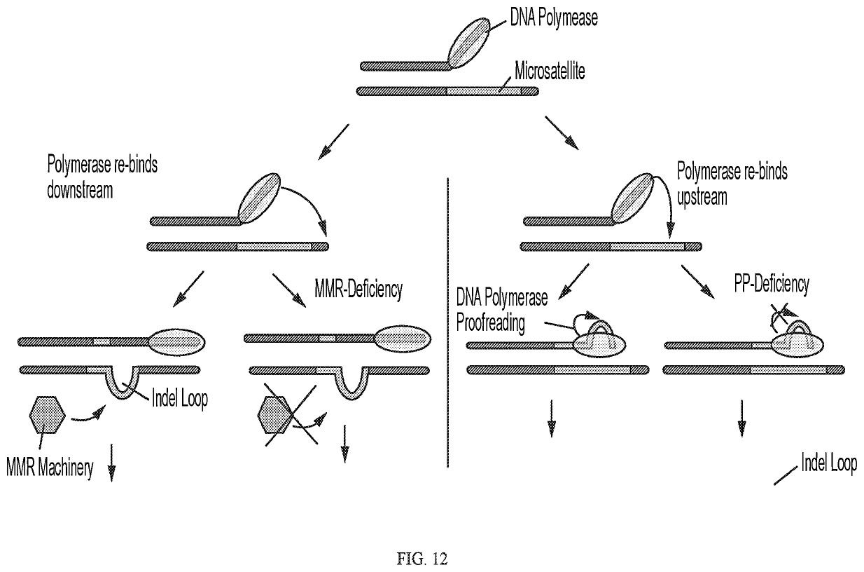

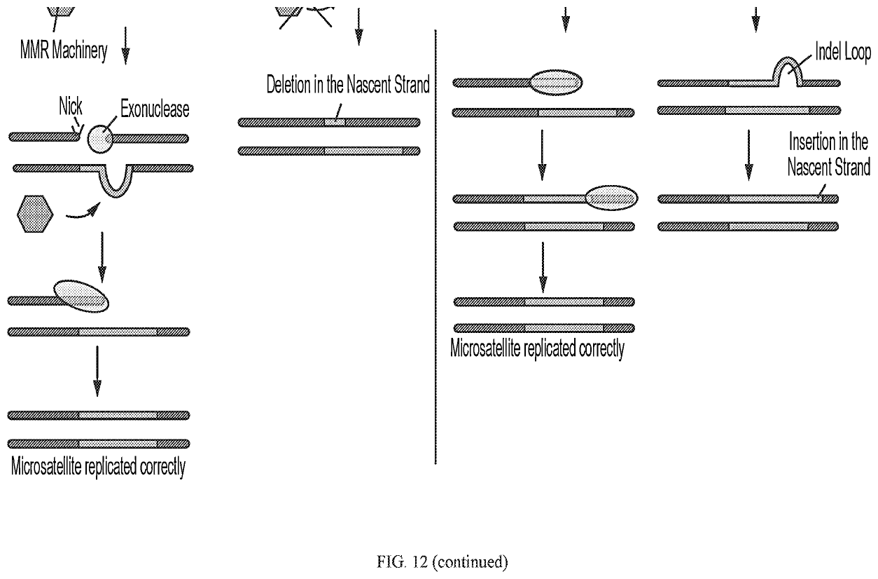

The data reveal that in addition to defects in MMR, loss of proofreading by replication-specific DNA polymerases was also associated with an increase in MS-indels. MS-indels produced by the deficiencies of the two replicative repair mechanisms have distinct characteristics. While MMRD generates mainly deletions (MS-sig1 and MS-sig3), PPD results predominantly in insertions (MS-sig2 and MS-sig4), suggesting a strand-bias repair process (FIG. 12).

Not intending to be bound by theory, it was hypothesized that the proofreading domain of the polymerase repairs extra bases on the nascent strand by changing the conformation of the DNA to push the substrate into the exonuclease domain of the polymerase. This would activate the exonucleolytic mode of DNA polymerase to excise the loop. Since mutations in the proofreading domain of the polymerase would prevent the DNA substrate from shifting into the exonuclease site, these remain unrepaired as replication continues, creating a permanent insertion. As larger insertions were not observed in PPD cancers, it was postulated that larger indel loops within the nascent strand do not fit into the exonuclease domain of the polymerase (FIG. 12). In contrast, the MMR system is more effective in detecting loops on the parental strand. These loops would result in deletions if unrepaired, which may translate into the surge of MS-sig1 and MS-sig3 in MMRD cancers in the cohort.

Another intriguing observation related to the role of MMR on the template strand was that larger deletions in adult MMRD cancers converged to a common length of microsatellites of −15 bp (FIG. 3C). Not intending to be bound by theory, this 15 bp convergence may be due to the size of the DNA binding domain of the polymerase complex being similar to the length of 15 bp, which is about 50 Å. Indeed, these large events can explain the ease of detection of MSI-H in adult MMRD cancers by the standard electrophoresis-based assay that specifically looks for long (≥3 bp) MS-indels. Furthermore, pediatric tumors lacked hotspots or commonly-mutated MS-loci even within specific tissues (FIG. 1E). In adults, MMRD cells can acquire MS-indels in the ACVR2A gene, which may lead to a selection advantage in the GI system, but not in other tissue types. Thus, the contrast between pediatric and adult cases may be due to the selective pressures that are present in different tissue types, although comparative analysis between pediatric and adult GI cancers still showed unique MS-indel profiles (FIG. 9B). Additionally, the distinct MS-deletions in adult and pediatric cancers might be due to unknown differences in DNA damage sensing or response mechanisms.

The biological observations described above can have a major clinical impact on the management of patients with replication repair deficient cancers. First, the standard methods for MSI classification (e.g. MIAS Promega) cannot detect MSI in most pediatric MMRD tumors (FIG. 1A) and possibly in tumors in which MMRD occurs late in carcinogenesis due to other mutational processes. The latter includes treatment-related MMRD cancers such as leukemias, gliomas and POLEmut-driven tumors, where MMRD occurs later, and are therefore falsely termed MSS. Both SNV- and MS-indel-based methods may be more sensitive and specific to classify such tumors correctly for management and potential genetic testing. Additionally, MS-sigs was able to differentiate between MMRD and PPD tumor genotypes (FIG. 4E), which has not been previously shown through SNV or the canonical MSI analysis method. The increased MMRDness signature in MSH2 and MLH1 mutant tumors (FIG. 4E) correlates with the more aggressive phenotype seen in Lynch Syndrome cases and the biological importance of the two proteins in the repair mechanism of the mutSα and mutSβ MMR complexes. The ability to use low-pass whole-genome sequencing from tumors, without their matched normal samples, can make MS-sigs the basis for an inexpensive tool for replication repair deficient classification. There has been a recent report that used deep sequencing of a panel of 277 MS-loci to diagnose CMMRD using blood DNA. However, the method of ultra low-pass whole-genome sequencing (ULP-WGS) at 0.1-0.5×coverage is less expensive per sample, and covers between 10% to 50% of the −23 million MS-loci, respectively. The ULP-WGS approach can be more sensitive and specific than deep sequencing of specific loci, depending on the depth of sequencing in each of the approaches. MS-sigs can also provide additive information to the clinical diagnosis, such as the POLEness signature that can predict response to immunotherapy (FIG. 5).

Secondly, the above data demonstrates that MS-sigs can be used to detect MMRD in non-malignant samples of individuals with CMMRD. The potential to detect MMRD from non-malignant samples, perhaps even before cancer development, has important clinical applications. In many cases, genetic diagnosis of CMMRD is difficult due to the large number of variants of unknown significance (VUS) and pseudogenes in the mismatch repair and DNA polymerase genes. Furthermore, MS-sigs was found to have extremely high sensitivity and specificity (both 100% in the cohort) in detecting MMRD in tumors and CMMRD in normal tissue. MS-sigs can distinguish functionally disruptive mutations from VUS's and pseudogenes in the MMR and polymerase genes, and can be conducted using low quantities (<150 ng) of unmatched DNA from FFPE-embedded or frozen tissue (FIG. 4D). It is suggest here that MS-sigs is an assay that can assess the MMRD phenotype from accessible, non-malignant tissues, which may become a frontline tool for the clinical diagnosis of replication repair deficient tumors and for monitoring patients at high risk.

Finally, the data provide further support to the notion that MS-indels may be strongly immunogenic in replication repair deficient hypermutant cancers. In the cohort, MS-sig based POLEness score, but not MMRDness, was a predictive biomarker for response to immune checkpoint inhibition. This adds to the finding which correlates MSI status to anti-PD-1 immunotherapy response. Since the cohort includes responders from brain and other tumor sites, which are not considered MSI-H using conventional methods, the robustness of Next-Generation Sequencing based MS-sig analysis may be superior to panel-based methods. Not intending to be bound by theory, the observations suggest that the single repeat insertions represented by the POLEness signature likely yield highly immunogenic neoantigens, which in turn result in a robust immunogenic response against replication repair deficient tumor cells.

In summary, the data provided herein adds a new dimension to the role of replication repair deficient in human cancer mutagenesis in the form of microsatellite maintenance and instability.

The following methods were employed in the above examples.

### Sample Collection and DNA Extraction for Whole Exome and Genome Sequencing of Replication Repair Deficient Tumors

As described in previous reports, patients with replication repair deficiency were routinely consented and registered into the International Replication Repair Consortium with written, informed consent. The study was conducted in accordance with the Canadian Tri-Council Policy Statement II (TCPS II), and was approved by the Institutional Research Ethics Board (REB approval number 1000048813), and all data were centralized in the Division of Haematology/Oncology at The Hospital for Sick Children (SickKids). Tumor and blood samples were collected from the Sickkids tumor bank, and diagnosis of replication repair deficiency was confirmed via sequencing and immunohistochemistry of the four MMR genes and sequencing of the POLE and POLD1 genes by a clinically approved laboratory. DNA was extracted using the Qiagen DNeasy Blood & Tissue kit for frozen tissues, and QIAamp DNA FFPE Tissue Kit for paraffin-embedded tissues.

### Whole Exome and Genome Sequencing Data of Pediatric Tumors in the KiCS Program

Whole exome and genome sequencing data of pediatric tumors were obtained through the Sickkids' Cancer Sequencing (KiCS) program. Detailed information about KiCS can be found at kicsprogram.com.

### Whole Exome Sequencing Data of Pediatric Brain Tumors

Informed consent was provided for patients with brain tumors treated at The Hospital for Sick Children (Toronto, Canada), and was approved by the institutional review board. DNA from the tumors and non-malignant samples were extracted for whole-exome sequencing.

### Whole Exome and Genome Sequencing Data and MSI Status Identification of Adult Tumors in the Cancer Genome Atlas Database

Whole exome sequencing and whole genome sequencing data for CRC (colorectal carcinoma), STAD (stomach adenoma), and UCEC (uterine corpus endometrial carcinoma) were downloaded from TCGA (The Cancer Genome Atlas), which were sequenced on the Illumina platform. As described in a previous report, their MSI status were annotated by TCGA.

Whole Exome Sequencing Data of Pediatric Neuroblastoma from the TARGET Database

Whole exome sequencing data of pediatric neuroblastoma was acquired from the Therapeutically Applicable Research to Generate Effective Treatments (TARGET) initiative.

### Microsatellite Instability Status of Pediatric Tumors Determined by the Microsatellite Instability Analysis System (Promega)

DNA extracted from tumor and matched normal tissues were quantified with Nanodrop (Thermo Scientific, Wilmington, DE), and amplified with Platinum™ Multiplex PCR Master Mix (ThermoFisher Scientific, Wilmington, DE) and MSI 10×Primer Pair Mix (Promega) in a Veriti™ 96-Well Thermal Cycler, as per the manufacturer's recommendations for PCR cycling conditions. The primer mix targets a panel of five mononucleotide loci, termed BAT-25, BAT-26, NR-21, NR-24, MONO-27, and following amplification, the products were run in a 3130 Genetic Analyzer for fluorescent capillary electrophoresis. Electrophoretograms were subsequently visualized using the Peak Scanner Software (v1.0, ThermoFisher Scientific, Wilmington, DE), and the highest peaks that were flanked by lower peaks were selected to be the representative alleles for each of the five loci in the panel. Each tumor-normal pair were compared by their allelic lengths. Tumors were considered MSI-High (MSI-H) if two or more loci were unstable (≥3 bp shift from the normal allele), MSI-Low (MSI-L) if one locus was unstable, and MSI stable (MSS) if all five loci were stable (<2 bp shift from the normal allele).

### Exome Sequencing of Replication Repair Deficient Pediatric Tumors

All high-throughput sequencing and mutation identification were performed at The Centre for Applied Genomics at the Hospital for Sick Children, as described in previous reports. Briefly, tumor and matched non-malignant tissue DNA were sequenced on an Illumina HiSeq2500 machine using Agilent's exome enrichment kit (Sure Select V4/V5; with >50% baits above 25×coverage). Processing into FASTQ files was done using CASAVA and/or HAS, which were aligned to UCSC's hg19 GRCh37 with BWA. The realignment and recalibration of aligned reads were done using SRMA and/or GATK and the Genome Analysis Toolkit26 (v1.1-28), respectively. Whole-exome tumor mutation burden was calculated using MuTect (v1.1.4) and filtered using dbSNP (v132), the 1000 Genomes Project (February 2012), a 69-sample Complete Genomics dataset, the Exome Sequencing Project (v6500), and the ExAc database for common single-nucleotide polymorphisms.

### Genome Sequencing of Pediatric Tumors

Whole genome sequencing was done using the Illumina HiSeq2500 or Illumina HiSeqX at ≥30×coverage, as well as on the Illumina NovaSeq6000 at 0.5× coverage. Read realignment and recalibration were conducted using the same pipeline as exome sequencing.

### Exomic and Genomic Microsatellite Definition and Identification and Mutation Calling via MSMuTect

The detailed methods of the MSMuTect algorithm was previously reported (Maruvka Y E, Mouw K W, Karlic R, Parasuraman P, Kamburov A, Polak P, et al. Analysis of somatic microsatellite indels identifies driver events in human tumors. Nat Biotechnol 2017; 35(10):951-9 doi 10.1038/nbt.3966). Briefly, repeats of five or more nucleotides were considered to be MS loci, and using the PHOBOS algorithm and the lobSTR approach, tumor and normal BAM files were aligned with their 5′ and 3′ flanking sequences. Each MS-locus allele was estimated using the empirical noise model, P_((j,m))\hat{ }Noise (k,m), which is the probability of observing a read with a microsatellite (MS) length k and motif m, where the true length of the allele is j with the motif m. This was used to call the MS alleles with the highest likelihood of being the true allele at each MS-locus. The MS alleles of each tumor and matched normal pair were called individually, which were compared to identify the mutations on the tumor MS-loci. The Akaike Information Criterion (AIC) score was assigned to both the tumor and normal models, and a threshold score that was determined by using simulated data was applied to make the final call of the MS-indel.

### Microsatellite Indel Signatures Discovered by SignatureAnalyzer

For WES samples, the number of MS-indels in the samples was quantified using different parameters, defined by the length of the microsatellite locus in the normal sample, the size of the indel (one base insertion/deletion, two base insertion/deletion, etc.), and the MS-locus motif (A- and C-repeats, FIGS. 2B and 7A). The BayesianNMF algorithm (SignatureAnalyzer) was then used with its default parameters to infer the different mutational processes operating in the samples. Other repeat motifs were not included, as they did not have sufficient mutational events.

### Calculation of MMRDness and POLEness Scores

Exome-Genome-wide microsatellite indels in each sample were quantified and segregated into deletions and insertions of up to three bases, and by locus size from five to 40 bases. The number of MS-indels in each sample was normalized to its own sum of total MS-indels. MMRDness scores were calculated by taking log 10 of the sum of the average proportions of one base deletions in loci sized from 10 to 15 bases. The POLEness scores were similarly calculated by taking log 10 of the sum of the average proportions of one base insertions in loci sized 5 to 6 bases. Both scores were normalized to the total number of MS-loci with the respective lengths.

### Whole Exome Sequencing Microsatellite Indel Quantification and Comparison

Exome-wide microsatellite indels for each tumor/normal pair were identified and quantified using MSMuTect (Maruvka Y E, Mouw K W, Karlic R, Parasuraman P, Kamburov A, Polak P, et al. Analysis of somatic microsatellite indels identifies driver events in human tumors. Nat Biotechnol 2017; 35(10):951-9 doi 10.1038/nbt.3966). The MSMuTect output was processed using the Rstudio graphical user interface (v1.1.447). The processing included summing the total number of MS-indels and calculating the length change of each microsatellite in each tumor compared to their matched normal samples. The medians number of MS-indels in the exome of each tumor type and in each MS-indel signature cluster were compared using the Mann-Whitney U-test in Rstudio, and statistical significance was considered at p<0.05.

### Whole Genome Sequencing Microsatellite Indel Quantification

Genome-wide microsatellite indels were summed for each sample according to the reference length of each locus and the change in size/length of the indel. Genome-wide deletions were identified and summed for both adult and pediatric samples, and were averaged for each deletion length. The mean and standard deviations were calculated using Rstudio (v1.1.447) at each deletion length in adult and pediatric samples independently. Fractions of indels based on initial and mutated loci size in adult cancers were calculated by dividing the number of events pertaining to a specific initial and mutated locus size by the sum of all MS-indels in all adult tumors. The same procedure was followed for pediatric cancers.

### MMRDness and POLEness Score Comparison Between Deficient MMR and Polymerase Proofreading

MMRDness medians were calculated for pediatric replication repair deficient tumors with the same mutated MMR gene, and compared between the four genes using the Mann-Whitney U-test. Similarly, POLEness medians were calculated for POLE or POLD1 mutants and compared.

### MMRDness and POLEness Score Calculation and Comparison for Ultra Low Coverage Whole Genome Sequencing Samples

MMRDness and POLEness scores were calculated as explained above for the ultra-low pass coverage sequencing samples. The tumors were separated according to their replication repair deficiency type (MMRP, PPD, MMRD, and MMRD&PPD), and medians of their MMRDness were calculated. Comparison of MMRDness between groups was done using the Mann-Whitney U-test. Germline samples were also segregated based on their replication repair deficiency statuses (MMRP, PPD, Lynch, CMMRD), and their MMRDness scores were compared using the same method as the tumors.

### Immunotherapy Response Thresholds for POLEness Scores

POLEness scores were determined from 28 tumors where WES was available, and from 22 of them that had WGS available. These tumors were collected from patients with replication repair deficient (RRD) cancers who underwent immunotherapy. POLEness scores were calculated as described above. For survival analysis, tumors were separated by their respective median POLEness scores (−2.92 in genome and −2.75 in exome). P values were determined using the Log Rank test.

### Code Availability

The software and pipelines used for data collection were the following: MSMuTect (Maruvka Y E, Mouw K W, Karlic R, Parasuraman P, Kamburov A, Polak P, et al. Analysis of somatic microsatellite indels identifies driver events in human tumors. Nat Biotechnol 2017; 35(10):951-9 doi 10.1038/nbt.3966), SignatureAnalyzer, MuTect. All data was analyzed using Rstudio (v1.1.447). All R packages and other statistical code used for analysis were: Scales, ggplot2, dplyr, Reshape2, RColorBrewer, ggpubr, tibble, epade, Plot3D, forcats, survival, survminer.

TABLE 1
MSI of germline MMRD tumors from the same patient. MSI of pediatric germline MMRD tumors from the
same patient showing different MSI. Samples with the same Patient.ID are tumors from the same patient.
NR24, NR21, BAT25, Mono27, and BAT26 are names of individual microsatellite loci that are used in
the Microsatellite Instability Analysis System (Promega).

| | MMR. | | | NR24. | NR24. | NR24. | NR21. | NR21. |
|---|---|---|---|---|---|---|---|---|
| | Gene. | Tumor. | MSI. | Allele. | Allele. | Allele. | Allele. | Allele. |
| Patient.ID | Mutated | Tissue | Status | 1 | 2 | 3 | 1 | 2 |
| MMR63 | PMS2 | GBM | MSI-H | 99 | 103 | NA | 130 | 132 |
| MMR63 | PMS2 | GBM | MSS | 102 | NA | NA | 130 | 132 |
| MMR63 | PMS2 | Adenoma | MSI-L | 102 | NA | NA | 132 | NA |
| | | with LGD | | | | | | |
| MMR84 | MSH6 | Colorectal | MSI-L | 101 | NA | NA | 131 | NA |
| | | Adenocarcinoma | | | | | | |
| MMR84 | MSH6 | Colorectal | MSS | 102 | NA | NA | 132 | NA |
| | | Adenocarcinoma | | | | | | |
| MMR84 | MSH6 | GI Polyp | MSI-H | 100 | 102 | NA | 131 | NA |
| MMR84 | MSH6 | Pre-B-Cell | MSS | 102 | NA | NA | 133 | NA |
| | | Acute | | | | | | |
| | | Lymphoblastic | | | | | | |
| | | Leukemia | | | | | | |
| MMR91 | PMS2 | Colorectal | MSS | 103 | NA | NA | 134 | NA |
| | | Adenocarcinoma | | | | | | |
| MMR91 | PMS2 | Tubular | MSI-H | 103 | NA | NA | 131 | NA |
| | | Adenoma | | | | | | |
| MMR91 | PMS2 | Colorectal | MSS | 103 | NA | NA | 134 | NA |
| | | Adenocarcinoma | | | | | | |
| MMR111 | PMS2 | GBM | MSS | 102 | NA | NA | 133 | NA |
| MMR111 | PMS2 | Ulna Tumor | MSI-H | 102 | 99 | NA | 127 | 132 |
| MMR160 | PMS2 | Colorectal | MSI-H | 102 | 99 | 96 | 134 | 131 |
| | | Adenocarcinoma | | | | | | |
| MMR160 | PMS2 | Colorectal | MSI-L | 103 | NA | NA | 134 | NA |
| | | Adenocarcinoma | | | | | | |
| MMR160 | PMS2 | GI Polyp | MSI-H | 100 | 97 | NA | 134 | 130 |
| MMR160 | PMS2 | Non- | MSS | 102 | NA | NA | 133 | NA |
| | | Hodgkin's | | | | | | |
| | | Lymphoma | | | | | | |
| MMR1267 | MSH2 | Adenoma | MSS | 101 | NA | NA | 133 | NA |
| | | with LGD | | | | | | |
| MMR1267 | MSH2 | Diffuse | MSI-H | 102 | NA | NA | 133 | 128 |
| | | Large B-Cell | | | | | | |
| | | Lymphoma | | | | | | |
| | | BAT25. | BAT25. | Mono27. | Mono27. | | BAT26. | BAT26. |
| | | Allele. | Allele. | Allele. | Allele. | Mono27 | Allele. | Allele. |
| | Patient.ID | 1 | 2 | 1 | 2 | Allele.3 | 1 | 2 |
| | MMR63 | 123 | NA | 139 | NA | NA | 178 | 180 |
| | MMR63 | 124 | NA | 140 | NA | NA | 181 | NA |
| | MMR63 | 125 | 122 | 139 | NA | NA | 180 | NA |
| | MMR84 | 119 | NA | 139 | NA | NA | 178 | 180 |
| | MMR84 | 123 | NA | 139 | NA | NA | 180 | NA |
| | MMR84 | 122 | NA | 140 | NA | NA | 178 | NA |
| | MMR84 | 122 | NA | 140 | NA | NA | 178 | NA |
| | MMR91 | 123 | NA | 139 | NA | NA | 181 | NA |
| | MMR91 | 120 | 124 | 139 | NA | NA | 179 | NA |
| | MMR91 | 123 | NA | 140 | NA | NA | 181 | NA |
| | MMR111 | 124 | NA | 139 | NA | NA | 180 | NA |
| | MMR111 | 122 | 118 | 139 | 135 | NA | 177 | NA |
| | MMR160 | 123 | NA | 138 | NA | NA | 179 | 175 |
| | MMR160 | 123 | NA | 139 | 136 | 147 | 180 | NA |
| | MMR160 | 122 | NA | 138 | NA | NA | 176 | NA |
| | MMR160 | 121 | NA | 138 | NA | NA | 179 | NA |
| | MMR1267 | 122 | NA | 141 | NA | NA | 179 | NA |
| | MMR1267 | 124 | NA | 141 | NA | NA | 180 | 171 |

GBM = Glioblastoma Multiforme,
LGD = Low-Grade Dysplasia,
GI = Gastrointestinal,
MSI-H = Microsatellite Instability High,
MSI-L = Microsatellite Instability Low,
MSS = Microsatellite stable.

TABLE 2
Proportion of MS-Loci mutated by Chromosome

| | Number of unique | Number of | Proportion of | |
|---|---|---|---|---|
| Chromosome | mutated MS-Loci | MS-Loci | MS-loci mutated | Group |
| chr1 | 165203 | 184399 | 0.9 | 1 |
| chr10 | 106952 | 106952 | 1 | 2 |
| chr11 | 89620 | 99824 | 0.9 | 3 |
| chr12 | 98131 | 109568 | 0.9 | 3 |
| chr13 | 67118 | 73454 | 0.91 | 3 |
| chr14 | 64447 | 71830 | 0.9 | 3 |
| chr15 | 60423 | 68154 | 0.89 | 3 |
| chr16 | 64214 | 72833 | 0.88 | 4 |
| chr17 | 67214 | 77181 | 0.87 | 4 |
| chr18 | 50916 | 57041 | 0.89 | 4 |
| chr19 | 56105 | 64557 | 0.87 | 4 |
| chr2 | 168407 | 186322 | 0.9 | 1 |
| chr20 | 48907 | 48907 | 1 | 4 |
| chr21 | 25411 | 28232 | 0.9 | 5 |
| chr22 | 28286 | 32147 | 0.88 | 5 |
| chr3 | 134726 | 150500 | 0.9 | 1 |
| chr4 | 129347 | 142702 | 0.91 | 1 |
| chr5 | 122297 | 136254 | 0.9 | 1 |
| chr6 | 118019 | 131052 | 0.9 | 2 |
| chr7 | 115933 | 129111 | 0.9 | 2 |
| chr8 | 98439 | 109343 | 0.9 | 2 |
| chr9 | 87150 | 96704 | 0.9 | 2 |
| chrX | 106600 | 117655 | 0.91 | 5 |
| chrY | 20577 | 21497 | 0.96 | 5 |

TABLE 3
List of most commonly mutated MS-loci in adult MMRD cancers and pediatric germline
MMRD cancers. The motif is the repeated unit of each microsatellite.

| | Length of | | Frequency | Percentage | | Region |
|---|---|---|---|---|---|---|
| Locus | Locus | Motif | of Cases | of Case | Gene | of Gene |
| CMMRD (n = 73) | | | | | | |
| chr3:164905649:164905659 | 11 | A | 14 | 0.192 | SLITRK3 | 3′UTR |
| chr6:55739712:55739725 | 14 | A | 14 | 0.192 | BMP5 | 5′UTR |
| chr3:113377482:113377492 | 11 | A | 15 | 0.205 | KIAA2018 | Intron |
| chr3:170715684:170715693 | 10 | A | 15 | 0.205 | SLC2A2 | Stop |
| | | | | | | Codon |
| | | | | | | Deletion |
| chr8:33356826:33356838 | 13 | A | 15 | 0.205 | TTI2 | Intron |
| chr14:94745084:94745094 | 11 | A | 16 | 0.219 | PPP4R4 | 3′UTR |
| STAD (n = 70) | | | | | | |
| chr17:33288434:33288444 | 11 | A | 39 | 0.557 | CCT6B | 5′UTR |
| chr2:211179766:211179776 | 11 | A | 41 | 0.586 | MYL1 | Stop |
| | | | | | | Codon |
| | | | | | | Deletion |
| chr3:130733047:130733057 | 11 | A | 42 | 0.6 | ASTE1 | Coding |
| chr3:113377482:113377492 | 11 | A | 49 | 0.7 | KIAA2018 | Intron |
| chr3:30691872:30691881 | 10 | A | 51 | 0.729 | TGFBR2 | Coding |
| chr2:148683686:148683693 | 8 | A | 54 | 0.77 | ACVR2A | Coding |
| COAD (n = 44) | | | | | | |
| chr4:13485808:13485815 | 8 | C | 18 | 0.409 | RAB28 | 5′UTR |
| chr9:97063726:97063736 | 11 | A | 18 | 0.409 | ZNF169 | 3′UTR |
| chr12:122242658:122242665 | 8 | C | 21 | 0.477 | SETD1B | Coding |
| chr17:56435161:56435167 | 7 | C | 21 | 0.477 | RNF43 | Coding |
| chr10:74653469:74653478 | 10 | A | 22 | 0.5 | OIT3 | 5′UTR |
| chr2:148683686:148683693 | 8 | A | 38 | 0.864 | ACVR2A | Coding |

3′UTR = 3′ Untranslated Region,
5′ UTR = 5′ Untranslated Region.

TABLE 4
Proportion of −1 deletions, +1 insertions, and deletions larger than 3 bases for each WGS sample used in the MS-indel
heatmap analyses

| | | | Fraction | Fraction | Fraction of | | |
|---|---|---|---|---|---|---|---|
| | | | of −1 | of +1 | deletions larger | | |
| | | | del/all | ins/all | than −3 bp/all | | MMR/POL |
| Sample | Cohort | Genotype | MS-indels | MS-indels | MS-indels | Tissue type | gene mutated |
| MD1337T2_MD1337B1 | Pediatric | PPD | 0.42 | 0.47 | 0.02 | Colorectal | POLE |
| | | | | | | adenocarcinoma | |
| MD138T2_MD138B1 | Pediatric | PPD | 0.67 | 0.18 | 0.03 | Colorectal | POLE |
| | | | | | | adenocarcinoma | |
| MD1362T1_MD1362B1 | Pediatric | PPD | 0.66 | 0.25 | 0.01 | Brain cancer | POLE |
| TCGA-BS-A0TC | Adult | MSS & PPD | 0.32 | 0.66 | 0.01 | UCEC | |
| TCGA-AX-A0J1 | Adult | MSI-H & | 0.59 | 0.14 | 0.07 | UCEC | |
| | | PPD | | | | | |
| TCGA-A5-A0G9 | Adult | MSI-H | 0.76 | 0.03 | 0.03 | UCEC | |
| TCGA-A5-A0GA | Adult | MSI-H | 0.68 | 0.03 | 0.07 | UCEC | |
| TCGA-AP-A051 | Adult | MSI-H | 0.62 | 0.09 | 0.08 | UCEC | |
| TCGA-AP-A054 | Adult | MSI-H | 0.53 | 0.06 | 0.17 | UCEC | |
| TCGA-AP-A0LD | Adult | MSI-H | 0.56 | 0.03 | 0.18 | UCEC | |
| TCGA-AP-A0LE | Adult | MSI-H | 0.62 | 0.06 | 0.11 | UCEC | |
| TCGA-AX-A05S | Adult | MSI-H | 0.54 | 0.03 | 0.18 | UCEC | |
| TCGA-B5-A11G | Adult | MSI-H | 0.49 | 0.17 | 0.14 | UCEC | |
| TCGA-B5-A11H | Adult | MSI-H | 0.47 | 0.02 | 0.25 | UCEC | |
| TCGA-BS-A0TE | Adult | MSI-H | 0.77 | 0.07 | 0.02 | UCEC | |
| TCGA-BR-4280 | Adult | MSI-H | 0.61 | 0.02 | 0.14 | STAD | |
| TCGA-BR-6452 | Adult | MSI-H | 0.48 | 0.01 | 0.26 | STAD | |
| TCGA-CG-4442 | Adult | MSI-H | 0.49 | 0.02 | 0.24 | STAD | |
| TCGA-CG-5723 | Adult | MSI-H | 0.46 | 0.01 | 0.26 | STAD | |
| TCGA-F1-6177 | Adult | MSI-H | 0.46 | 0.03 | 0.29 | STAD | |
| TCGA-A6-3809 | Adult | MSI-H | 0.35 | 0.02 | 0.38 | COAD | |
| TCGA-A6-6780 | Adult | MSI-H | 0.45 | 0.02 | 0.30 | COAD | |
| TCGA-A6-6781 | Adult | MSI-H | 0.33 | 0.01 | 0.43 | COAD | |
| TCGA-AA-3516 | Adult | MSI-H | 0.39 | 0.01 | 0.36 | COAD | |
| TCGA-AA-3518 | Adult | MSI-H | 0.44 | 0.02 | 0.28 | COAD | |
| TCGA-AA-A00R | Adult | MSI-H | 0.37 | 0.01 | 0.38 | COAD | |
| TCGA-AA-A01R | Adult | MSI-H | 0.51 | 0.03 | 0.22 | COAD | |
| TCGA-AD-6964 | Adult | MSI-H | 0.41 | 0.02 | 0.29 | COAD | |
| TCGA-AD-A5EJ | Adult | MSI-H | 0.42 | 0.05 | 0.31 | COAD | |
| TCGA-AZ-6601 | Adult | MSI-H | 0.54 | 0.02 | 0.17 | COAD | |
| TCGA-D5-6540 | Adult | MSI-H | 0.44 | 0.04 | 0.30 | COAD | |
| TCGA-QG-A5Z2 | Adult | MSI-H | 0.42 | 0.02 | 0.27 | COAD | |
| MD66T1_MD66B1 | Pediatric | MMRD & | 0.28 | 0.35 | 0.07 | Other cancer | MSH6 |
| | | PPD | | | | | |
| D1755_MMR111B1 | Pediatric | MMRD & | 0.28 | 0.30 | 0.20 | Colorectal | PMS2 |
| | | PPD | | | | cancer- | |
| | | | | | | metastasis | |
| M160T7_MD160B1 | Pediatric | MMRD & | 0.41 | 0.27 | 0.11 | Colorectal | PMS2 |
| | | PPD | | | | adenocarcinoma | |
| MD2T27_MMR2B1 | Pediatric | MMRD & | 0.56 | 0.19 | 0.05 | Colorectal | MLH1 |
| | | PPD | | | | adenocarcinoma | |
| D1762_M125B1 | Pediatric | MMRD & | 0.61 | 0.07 | 0.08 | Colorectal | MSH6 |
| | | PPD | | | | adenocarcinoma | |
| MD1273T2_MD1273B1 | Pediatric | MMRD & | 0.10 | 0.84 | 0.00 | Brain cancer | PMS2 |
| | | PPD | | | | | |
| D132 | Pediatric | MMRD & | 0.15 | 0.82 | 0.00 | Brain cancer | PMS2 |
| | | PPD | | | | | |
| D1121 | Pediatric | MMRD & | 0.15 | 0.81 | 0.00 | Brain cancer | PMS2 |
| | | PPD | | | | | |
| MD1273T26_MD1273B1 | Pediatric | MMRD & | 0.11 | 0.77 | 0.00 | Brain cancer | PMS2 |
| | | PPD | | | | | |
| MD1273T25_MD1273B1 | Pediatric | MMRD & | 0.11 | 0.75 | 0.00 | Brain cancer | PMS2 |
| | | PPD | | | | | |
| D1244 | Pediatric | MMRD & | 0.22 | 0.74 | 0.00 | Brain cancer | PMS2 |
| | | PPD | | | | | |
| D1243 | Pediatric | MMRD & | 0.25 | 0.72 | 0.00 | Brain cancer | PMS2 |
| | | PPD | | | | | |
| D1410_MMR111B1 | Pediatric | MMRD & | 0.17 | 0.69 | 0.01 | Brain cancer | PMS2 |
| | | PPD | | | | | |
| MD134T13_MD134B1 | Pediatric | MMRD & | 0.21 | 0.68 | 0.01 | Brain cancer | PMS2 |
| | | PPD | | | | | |
| MD134T11_MD134B1 | Pediatric | MMRD & | 0.22 | 0.67 | 0.01 | Brain cancer | PMS2 |
| | | PPD | | | | | |
| MD1341T3_MD1341B1 | Pediatric | MMRD & | 0.29 | 0.65 | 0.00 | Brain cancer | PMS2 |
| | | PPD | | | | | |
| D1805_2_MMR101B1 | Pediatric | MMRD & | 0.23 | 0.65 | 0.01 | Brain cancer | PMS2 |
| | | PPD | | | | | |
| D1805_1_MMR101B1 | Pediatric | MMRD & | 0.24 | 0.65 | 0.00 | Brain cancer | PMS2 |
| | | PPD | | | | | |
| D134 | Pediatric | MMRD & | 0.34 | 0.63 | 0.00 | Brain cancer | MLH1 |
| | | PPD | | | | | |
| MD134T10_MD134B1 | Pediatric | MMRD & | 0.26 | 0.63 | 0.01 | Brain cancer | PMS2 |
| | | PPD | | | | | |
| D1763_2_MMR117B1 | Pediatric | MMRD & | 0.25 | 0.63 | 0.01 | Brain cancer | PMS2 |
| | | PPD | | | | | |
| D1764_MMR128B1 | Pediatric | MMRD & | 0.28 | 0.62 | 0.01 | Brain cancer | MSH2 |
| | | PPD | | | | | |
| D1807_1_MMR128B1 | Pediatric | MMRD & | 0.29 | 0.58 | 0.01 | Brain cancer | MSH2 |
| | | PPD | | | | | |
| D1763_1_MMR117B1 | Pediatric | MMRD & | 0.24 | 0.58 | 0.02 | Brain cancer | PMS2 |
| | | PPD | | | | | |
| D1119 | Pediatric | MMRD & | 0.37 | 0.58 | 0.00 | Brain cancer | PMS2 |
| | | PPD | | | | | |
| D1424_MMR117B1 | Pediatric | MMRD & | 0.29 | 0.57 | 0.02 | Brain cancer | PMS2 |
| | | PPD | | | | | |
| MD1385T2_MD1385B1 | Pediatric | MMRD & | 0.33 | 0.55 | 0.01 | Brain cancer | PMS2 |
| | | PPD | | | | | |
| D1423-2_MMR120B1 | Pediatric | MMRD & | 0.40 | 0.52 | 0.01 | Brain cancer | MSH6 |
| | | PPD | | | | | |
| D1120 | Pediatric | MMRD & | 0.38 | 0.52 | 0.01 | Brain cancer | PMS2 |
| | | PPD | | | | | |
| D1423-1_MMR120B1 | Pediatric | MMRD & | 0.39 | 0.51 | 0.01 | Brain cancer | MSH6 |
| | | PPD | | | | | |
| MD134T12_MD134B1 | Pediatric | MMRD & | 0.31 | 0.48 | 0.04 | Brain cancer | PMS2 |
| | | PPD | | | | | |
| D1577 | Pediatric | MMRD & | 0.45 | 0.47 | 0.01 | Brain cancer | MSH6 |
| | | PPD | | | | | |
| D1807_2_MMR128B1 | Pediatric | MMRD & | 0.46 | 0.45 | 0.01 | Brain cancer | MSH2 |
| | | PPD | | | | | |
| D1804_2_MMR128B1 | Pediatric | MMRD & | 0.43 | 0.44 | 0.01 | Brain cancer | MSH2 |
| | | PPD | | | | | |
| D1804_3_MMR128B1 | Pediatric | MMRD & | 0.44 | 0.43 | 0.01 | Brain cancer | MSH2 |
| | | PPD | | | | | |
| D1806_MMR128B1 | Pediatric | MMRD & | 0.48 | 0.43 | 0.01 | Brain cancer | MSH2 |
| | | PPD | | | | | |
| D1804_1_MMR128B1 | Pediatric | MMRD & | 0.44 | 0.42 | 0.02 | Brain cancer | MSH2 |
| | | PPD | | | | | |
| D1122 | Pediatric | MMRD & | 0.62 | 0.31 | 0.01 | Brain cancer | MSH6 |
| | | PPD | | | | | |
| MD80T2_MD80B1 | Pediatric | MMRD & | 0.66 | 0.26 | 0.01 | Brain cancer | MSH6 |
| | | PPD | | | | | |
| D1144 | Pediatric | MMRD & | 0.77 | 0.17 | 0.01 | Brain cancer | MSH6 |
| | | PPD | | | | | |
| D849 | Pediatric | MMRD | 0.88 | 0.05 | 0.00 | T-cell | MSH6 |
| | | | | | | lymphoma | |
| D1118 | Pediatric | MMRD | 0.80 | 0.09 | 0.01 | Other cancer | MLH1 |
| D204 | Pediatric | MMRD | 0.72 | 0.18 | 0.03 | Low grade | PMS2 |
| | | | | | | glioma | |
| D1167 | Pediatric | MMRD | 0.73 | 0.23 | 0.00 | Brain cancer | MSH6 |
| MD1268T2_MD1268B1 | Pediatric | MMRD | 0.71 | 0.18 | 0.01 | Brain cancer | MSH6 |
| MD139T2_M139B1 | Pediatric | MMRD | 0.66 | 0.22 | 0.02 | Brain cancer | MSH6 |

TABLE 5
Detection of MMRD in tumors by MMRDness and POLEness. Ness-Scores and tumor
types of the four acquired MMRD tumors in germline MMRP patients.
The deficient MMR genes were confirmed via genotype analysis.

| | | Change in | | Change in | | | |
|---|---|---|---|---|---|---|---|
| | | MMRDnes | | POLEness | | | |
| | | s (from | | (from | | MMR | |
| | MMRD | matched | | matched | | Gene | |
| KiCS ID | ness | normal) | POLEness | normal) | Tissue | Mutated | TMB |
| K18 | −1.05 | 0.23 | −3.1 | 0.01 | Malignant | MSH2 | 3.3 |
| | | | | | peripheral nerve | | |
| | | | | | sheath tumor | | |
| | | | | | (MPNST) | | |
| K62 | −0.89 | 0.24 | −3.08 | 0.03 | Glioblastoma | MSH2 | 7.2 |
| | | | | | Multiforme | | |
| | | | | | (GBM) | | |
| K83 | −1.06 | 0.22 | −3.11 | 0.01 | Metastatic | MLH1 | 4.3 |
| | | | | | Osteosarcoma | | |
| K156 | −0.82 | 0.45 | −3.08 | 0.03 | Colorectal | EPCAM/ | 17.6 |
| | | | | | Adenocarcinom | MSH2 | |
| | | | | | a (CRC) | | |

TABLE 6
WGS Ness Scores

| | | | | | | Polymerase Driver | | |
|---|---|---|---|---|---|---|---|---|
| | | | | | | Including Passenger | | |
| Sample | Donor | Tissue type | Tissue category | MMR | RRD Type | Info | MMRDness | POLEness |
| D1120 | MMR63 | GBM | Brain tumor | CMMRD | MMRD & PPD | POLD1 | −1.092051 | −2.7905659 |
| D1119 | MMR62 | PNET | Brain tumor | CMMRD | MMRD & PPD | POLE | −1.0594597 | −2.847022 |
| D1764 | MMR128 | GBM | Brain tumor | CMMRD | MMRD & PPD | POLD1 | −0.8995259 | −2.5442231 |
| D1807_1 | MMR128 | GBM | Brain tumor | CMMRD | MMRD & PPD | POLD1 | −0.8834019 | −2.5231048 |
| D1122 | MMR68 | GBM | Brain tumor | CMMRD | MMRD & PPD | POLD1 | −1.0620619 | −2.9448141 |
| MD1273T25 | MMR1273 | GBM | Brain tumor | CMMRD | MMRD & PPD | POLD1 | −1.0444482 | −2.1840413 |
| MD1273T26 | MMR1273 | GBM | Brain tumor | CMMRD | MMRD & PPD | POLD1 | −1.0714141 | −2.2594764 |
| MD134T10 | MMR134 | GBM | Brain tumor | CMMRD | MMRD & PPD | POLD1 | −0.9659468 | −2.4307557 |
| MD134T11 | MMR134 | GBM | Brain tumor | CMMRD | MMRD & PPD | POLD1 | −0.9777379 | −2.3606967 |
| MD134T12 | MMR134 | GBM | Brain tumor | CMMRD | MMRD & PPD | POLD1 | −1.1249672 | −2.789372 |
| MD134T13 | MMR134 | GBM | Brain tumor | CMMRD | MMRD & PPD | POLD1 | −0.9781798 | −2.3611674 |
| MD1337T2 | MMR1337 | GI | GI tumor | PPD | PPD | POLE | −1.2227194 | −2.8424301 |
| | | Adenocarcinoma | | | | | | |
| D1121 | MMR63 | GBM | Brain tumor | CMMRD | MMRD & PPD | POLE | −1.1568575 | −2.8763894 |
| D1424 | MMR117 | GBM | Brain tumor | CMMRD | MMRD & PPD | POLE | −1.019148 | −2.9289699 |
| D1144 | MMR8 | GBM | Brain tumor | CMMRD | MMRD & PPD | POLE | −1.0828483 | −2.9973067 |
| D1755 | MMR111 | GI | GI tumor | CMMRD | MMRD & PPD | POLE | −0.7572027 | −2.3469542 |
| | | Adenocarcinoma | | | | | | |
| D1243 | MMR100 | GBM | Brain tumor | CMMRD | MMRD & PPD | POLE | −1.067674 | −2.7553522 |
| D1763_1 | MMR117 | GBM | Brain tumor | CMMRD | MMRD & PPD | POLE | −0.9567831 | −2.737327 |
| D1244 | MMR101 | GBM | Brain tumor | CMMRD | MMRD & PPD | POLE | −1.0410407 | −2.7197174 |
| D132 | MMR1 | Anaplastic PXA | Brain tumor | CMMRD | MMRD & PPD | POLE | −1.0787561 | −2.6287671 |
| D134 | MMR2 | Anaplastic | Brain tumor | CMMRD | MMRD & PPD | POLE | −1.0467219 | −2.8473709 |
| | | Astrocytoma | | | | | | |
| D1410 | MMR111 | GBM | Brain tumor | CMMRD | MMRD & PPD | POLE | −0.9728396 | −2.7157151 |
| D1423_1 | MMR120 | GBM | Brain tumor | CMMRD | MMRD & PPD | POLE | −1.0084542 | −2.9638572 |
| D1423-2 | MMR120 | GBM | Brain tumor | CMMRD | MMRD & PPD | POLE | −0.9999758 | −2.9624661 |
| D1577 | MMR139 | GBM | Brain tumor | CMMRD | MMRD & PPD | POLE | −1.0563803 | −2.9514435 |
| D1762 | MMR125 | GI | GI tumor | CMMRD | MMRD & PPD | POLE | −0.7985736 | −2.8722882 |
| | | Adenocarcinoma | | | | | | |
| D1804 | MMR128 | GBM | Brain tumor | CMMRD | MMRD & PPD | POLE | −0.8623038 | −2.8139898 |
| D1804_2 | MMR128 | GBM | Brain tumor | CMMRD | MMRD & PPD | POLE | −0.8512056 | −2.827599 |
| D1804_3 | MMR128 | GBM | Brain tumor | CMMRD | MMRD & PPD | POLE | −0.8687101 | −2.8507273 |
| D1805 | MMR101 | GBM | Brain tumor | CMMRD | MMRD & PPD | POLE | −0.9172188 | −2.6371271 |
| D1805_2 | MMR101 | GBM | Brain tumor | CMMRD | MMRD & PPD | POLE | −0.9010738 | −2.6278233 |
| D1806 | MMR128 | GBM | Brain tumor | CMMRD | MMRD & PPD | POLE | −0.853214 | −2.8251898 |
| D1807_2 | MMR128 | GBM | Brain tumor | CMMRD | MMRD & PPD | POLE | −0.8587813 | −2.7960957 |
| M160T7 | MMR160 | GI | GI tumor | CMMRD | MMRD & PPD | POLE | −0.8838056 | −2.7226543 |
| | | Adenocarcinoma | | | | | | |
| MD1362T1 | MMR1362 | GBM | Brain tumor | PPD | MMRD & PPD | POLE | −1.091807 | −2.8207497 |
| MD138T2 | MMR138 | GI | GI tumor | PPD | MMRD & PPD | POLE | −1.000724 | −2.7379862 |
| | | Adenocarcinoma | | | | | | |
| MD1385T2 | MMR1385 | GBM | Brain tumor | CMMRD | MMRD & PPD | POLE | −1.000724 | −2.7379862 |
| MD2T27 | MMR2 | GI | GI tumor | CMMRD | MMRD & PPD | POLE | −0.925385 | −2.8848945 |
| | | Adenocarcinoma | | | | | | |
| D1118 | MMR79 | Wilms tumor | Other tumor | CMMRD | MMRD | N/A | −0.9541582 | −3.0905522 |
| D1167 | MMR87 | GBM | Brain tumor | CMMRD | MMRD | N/A | −1.0916952 | −3.0563176 |
| D1578 | MMR139 | GBM | Brain tumor | CMMRD | MMRD | N/A | −1.1540805 | −3.1127646 |
| D849 | MMR8 | T-cell | Hematological | CMMRD | MMRD | N/A | −0.9425836 | −3.0886048 |
| | | Lymphoma | cancer | | | | | |
| M1268T2 | MMR1268 | Anaplastic | Brain tumor | CMMRD | MMRD | N/A | −1.0686937 | −3.0912442 |
| | | Astrocytoma | | | | | | |
| K_169 + T_ | | ALL | Hematological | MMR | MMRP | N/A | −1.1857871 | −3.0517807 |
| 319629 + N_ | | | Cancer | Proficient | | | | |
| 319104 | | | | | | | | |
| K_14 + T_ | | ALL | Hematological | MMR | MMRP | N/A | −1.2140867 | −3.1117035 |
| 328066 + N_ | | | Cancer | Proficient | | | | |
| 328067 | | | | | | | | |
| K_30 + T_ | | Gliosarcoma | Brain tumor | MMR | MMRP | N/A | −1.2230616 | −3.0816647 |
| 273368 + N_ | | | | Proficient | | | | |
| 274027 | | | | | | | | |
| K_92 + T_ | | Neuroblastoma | Other tumor | MMR | MMRP | N/A | −1.2241608 | −3.0823576 |
| 329766 + N_ | | | | Proficient | | | | |
| 301002 | | | | | | | | |
| K_92 + T_ | | Neuroblastoma | Other tumor | MMR | MMRP | N/A | −1.2254858 | −3.0827495 |
| 319682 + N_ | | | | Proficient | | | | |
| 301062 | | | | | | | | |
| K_171 + T_ | | ALL | Hematological | MMR | MMRP | N/A | −1.2394623 | −3.0920527 |
| 319910 + N_ | | | Cancer | Proficient | | | | |
| 320118 | | | | | | | | |
| K_194 + T_ | | Osteosarcoma | Other tumor | MMR | MMRP | N/A | −1.2529771 | −3.1030454 |
| 328185 + N_ | | | | Proficient | | | | |
| 328798 | | | | | | | | |
| K_197 + T_ | | Neuroblastoma | Other tumor | MMR | MMRP | N/A | −1.2546286 | −3.1075423 |
| 326711 + N_ | | | | Proficient | | | | |
| 328003 | | | | | | | | |
| K_8 + T_ | | Colorectal | GI tumor | MMR | MMRP | N/A | −1.2564478 | −3.0859791 |
| 289185 + N_ | | Cancer | | Proficient | | | | |
| 321323 | | | | | | | | |
| K_10 + T_ | | Osteosarcoma | Other tumor | MMR | MMRP | N/A | −1.2572557 | −3.116143 |
| 270645 + N_ | | | | Proficient | | | | |
| 271112 | | | | | | | | |
| K_88 + T_ | | Osteosarcoma | Other tumor | MMR | MMRP | N/A | −1.2573091 | −3.1026674 |
| 298840 + N_ | | | | Proficient | | | | |
| 300799 | | | | | | | | |
| K_86 + T_ | | Ovarian Cancer | Other tumor | MMR | MMRP | N/A | −1.2582874 | −3.1085148 |
| 298838 + N_ | | | | Proficient | | | | |
| 299337 | | | | | | | | |
| K_39 + T_ | | Rhabdomyo- | Other tumor | MMR | MMRP | N/A | −1.258334 | −3.1065932 |
| 297368 + N_ | | sarcoma | | Proficient | | | | |
| 298866 | | | | | | | | |
| K_154 + T_ | | Kidney tumor | Other tumor | MMR | MMRP | N/A | −1.259336 | −3.1163735 |
| 316299 + N_ | | | | Proficient | | | | |
| 316626 | | | | | | | | |
| K_76 + T_ | | Neuroblastoma | Other tumor | MMR | MMRP | N/A | −1.2595349 | −3.0953268 |
| 299020 + N_ | | | | Proficient | | | | |
| 298310 | | | | | | | | |
| K_41 + T_ | | Sarcoma | Other tumor | MMR | MMRP | N/A | −1.260134 | −3.1096576 |
| 317512 + N_ | | | | Proficient | | | | |
| 316866 | | | | | | | | |
| K_44 + T_ | | Liver cancer | Other tumor | MMR | MMRP | N/A | −1.2603597 | −3.1066466 |
| 285046 + N_ | | | | Proficient | | | | |
| 287846 | | | | | | | | |
| K_41 + T_ | | Sarcoma | Other tumor | MMR | MMRP | N/A | −1.2604971 | −3.1091108 |
| 317510 + N_ | | | | Proficient | | | | |
| 316866 | | | | | | | | |
| K_15 + T_ | | Rhabdomyo- | Other tumor | MMR | MMRP | N/A | −1.2606098 | −3.1147659 |
| 288017 + N_ | | sarcoma | | Proficient | | | | |
| 284386 | | | | | | | | |
| K_76 + T_ | | Neuroblastoma | Other tumor | MMR | MMRP | N/A | −1.2607485 | −3.0948929 |
| 303115 + N_ | | | | Proficient | | | | |
| 298369 | | | | | | | | |
| K_125 + T_ | | Sarcoma | Other tumor | MMR | MMRP | N/A | −1.2608195 | −3.1085619 |
| 309277 + N_ | | | | Proficient | | | | |
| 316071 | | | | | | | | |
| K_46 + T_ | | Not available | Not available | MMR | MMRP | N/A | −1.2610236 | −3.1057367 |
| 292280 + N_ | | | | Proficient | | | | |
| 293843 | | | | | | | | |
| K_154 + T_ | | Kidney tumor | Other tumor | MMR | MMRP | N/A | −1.2616995 | −3.1161807 |
| 326256 + N_ | | | | Proficient | | | | |
| 316626 | | | | | | | | |
| K_17 + T_ | | Neuroblastoma | Other tumor | MMR | MMRP | N/A | −1.2628763 | −3.1050773 |
| 271603 + N_ | | | | Proficient | | | | |
| 271117 | | | | | | | | |
| K_172 + T_ | | Sarcoma | Other tumor | MMR | MMRP | N/A | −1.2631113 | −3.1101074 |
| 321979 + N_ | | | | Proficient | | | | |
| 321967 | | | | | | | | |
| K_33 + T_ | | Thyroid cancer | Other tumor | MMR | MMRP | N/A | −1.2634082 | −3.1024586 |
| 319631 + N_ | | | | Proficient | | | | |
| 319630 | | | | | | | | |
| K_43 + T_ | | Kidney tumor | Other tumor | MMR | MMRP | N/A | −1.2634188 | −3.1142866 |
| 290134 + N_ | | | | Proficient | | | | |
| 287731 | | | | | | | | |
| K_17 + T_ | | Neuroblastoma | Other tumor | MMR | MMRP | N/A | −1.263641 | −3.1067313 |
| 270651 + N_ | | | | Proficient | | | | |
| 271117 | | | | | | | | |
| K_179 + T_ | | Osteosarcoma | Other tumor | MMR | MMRP | N/A | −1.2648243 | −3.1112066 |
| 322516 + N_ | | | | Proficient | | | | |
| 323141 | | | | | | | | |
| K_13 + T_ | | Astrocytoma | Brain tumor | MMR | MMRP | N/A | −1.2654979 | −3.1074204 |
| 319375 + N_ | | | | Proficient | | | | |
| 319374 | | | | | | | | |
| K_99 + T_ | | Neuroblastoma | Other tumor | MMR | MMRP | N/A | −1.2655012 | −3.1149362 |
| 301444 + N_ | | | | Proficient | | | | |
| 301286 | | | | | | | | |
| K_123 + T_ | | Myofibroma | Other tumor | MMR | MMRP | N/A | −1.2657369 | −3.1126547 |
| 308934 + N_ | | | | Proficient | | | | |
| 308089 | | | | | | | | |
| K_2 + T_ | | Yolk sac tumor | Other tumor | MMR | MMRP | N/A | −1.2659454 | −3.1195308 |
| 321321 + N_ | | | | Proficient | | | | |
| 321322 | | | | | | | | |
| K_10 + T_ | | Osteosarcoma | Other tumor | MMR | MMRP | N/A | −1.2660226 | −3.1024659 |
| 270647 + N_ | | | | Proficient | | | | |
| 271112 | | | | | | | | |
| K_121 + T_ | | ALL | Hematological | MMR | MMRP | N/A | −1.2660442 | −3.1143505 |
| 314881 + N_ | | | Cancer | Proficient | | | | |
| 311680 | | | | | | | | |
| K_25 + T_ | | Rhabdomyo- | Other tumor | MMR | MMRP | N/A | −1.2663518 | −3.1154176 |
| 307284 + N_ | | sarcoma | | Proficient | | | | |
| 273308 | | | | | | | | |
| K_184 + T_ | | Osteosarcoma | Other tumor | MMR | MMRP | N/A | −1.2665008 | −3.1090291 |
| 324069 + N_ | | | | Proficient | | | | |
| 324076 | | | | | | | | |
| K_167 + T_ | | Glioma | Brain tumor | MMR | MMRP | N/A | −1.2668681 | −3.1242924 |
| 318530 + N_ | | | | Proficient | | | | |
| 318749 | | | | | | | | |
| K_45 + T_ | | Leukemia | Hematological | MMR | MMRP | N/A | −1.2669089 | −3.114359 |
| 288555 + N_ | | | Cancer | Proficient | | | | |
| 288283 | | | | | | | | |
| K_165 + T_ | | Neuroblastoma | Other tumor | MMR | MMRP | N/A | −1.2674602 | −3.119166 |
| 317832 + N_ | | | | Proficient | | | | |
| 317981 | | | | | | | | |
| K_101 + T_ | | Sarcoma | Other tumor | MMR | MMRP | N/A | −1.2678703 | −3.1071018 |
| 303761 + N_ | | | | Proficient | | | | |
| 303763 | | | | | | | | |
| K_10 + T_ | | Osteosarcoma | Other tumor | MMR | MMRP | N/A | −1.268326 | −3.1155313 |
| 270646 + N_ | | | | Proficient | | | | |
| 271112 | | | | | | | | |
| K_15 + T_ | | Rhabdomyo- | Other tumor | MMR | MMRP | N/A | −1.2686324 | −3.1152114 |
| 283113 + N_ | | sarcoma | | Proficient | | | | |
| 284386 | | | | | | | | |
| K_126 + T_ | | Not available | Not available | MMR | MMRP | N/A | −1.2689822 | −3.1137982 |
| 328068 + N_ | | | | Proficient | | | | |
| 315585 | | | | | | | | |
| K_85 + T_ | | Rhabdomyo- | Other tumor | MMR | MMRP | N/A | −1.269075 | −3.1200711 |
| 299826 + N_ | | sarcoma | | Proficient | | | | |
| 297043 | | | | | | | | |
| K_84 + T_ | | T-cell lymphoma | Hematological | MMR | MMRP | N/A | −1.2706348 | −3.1174616 |
| 300714 + N_ | | | Cancer | Proficient | | | | |
| 304479 | | | | | | | | |
| K_40 + T_ | | Neuroblastoma | Other tumor | MMR | MMRP | N/A | −1.2707999 | −3.0331789 |
| 286739 + N_ | | | | Proficient | | | | |
| 286737 | | | | | | | | |
| K_42 + T_ | | Ependymoma | Other tumor | MMR | MMRP | N/A | −1.2713774 | −3.1151807 |
| 284388 + N_ | | | | Proficient | | | | |
| 284385 | | | | | | | | |
| K_16 + T_ | | Astrocytoma | Brain tumor | MMR | MMRP | N/A | −1.2717625 | −3.111676 |
| 270650 + N_ | | | | Proficient | | | | |
| 271115 | | | | | | | | |
| K_198 + T_ | | Sarcoma | Other tumor | MMR | MMRP | N/A | −1.2721267 | −3.1204166 |
| 327862 + N_ | | | | Proficient | | | | |
| 327864 | | | | | | | | |
| K_38 + T_ | | Liver cancer | Other tumor | MMR | MMRP | N/A | −1.2730545 | −3.118887 |
| 295256 + N_ | | | | Proficient | | | | |
| 286900 | | | | | | | | |
| K_7 + T_ | | Neuroblastoma | Other tumor | MMR | MMRP | N/A | −1.2735932 | −3.0770021 |
| 302948 + N_ | | | | Proficient | | | | |
| 300063 | | | | | | | | |
| K_95 + T_ | | Leukemia | Hematological | MMR | MMRP | N/A | −1.2737789 | −3.1129269 |
| 300240 + N_ | | | Cancer | Proficient | | | | |
| 300153 | | | | | | | | |
| K_78 + T_ | | Ependymoma | Other tumor | MMR | MMRP | N/A | −1.2738678 | −3.1167748 |
| 297044 + N_ | | | | Proficient | | | | |
| 297445 | | | | | | | | |
| K_87 + T_ | | Sarcoma | Other tumor | MMR | MMRP | N/A | −1.2740555 | −3.1198678 |
| 297229 + N_ | | | | Proficient | | | | |
| 297377 | | | | | | | | |
| K_87 + T_ | | Sarcoma | Other tumor | MMR | MMRP | N/A | −1.27477 | −3.1130636 |
| 298485 + N_ | | | | Proficient | | | | |
| 297377 | | | | | | | | |
| K_168 + T_ | | Neuroblastoma | Other tumor | MMR | MMRP | N/A | −1.2754455 | −3.1264665 |
| 319321 + N_ | | | | Proficient | | | | |
| 318878 | | | | | | | | |
| K_106 + T_ | | Ependymoma | Brain tumor | MMR | MMRP | N/A | −1.2754716 | −3.1204239 |
| 302802 + N_ | | | | Proficient | | | | |
| 302957 | | | | | | | | |
| K_91 + T_ | | Neuroblastoma | Other tumor | MMR | MMRP | N/A | −1.2764992 | −3.1187744 |
| 301892 + N_ | | | | Proficient | | | | |
| 301460 | | | | | | | | |
| K_32 + T_ | | Neuroblastoma | Other tumor | MMR | MMRP | N/A | −1.2773485 | −3.1063426 |
| 273811 + N_ | | | | Proficient | | | | |
| 274026 | | | | | | | | |
| K_38 + T_ | | Liver cancer | Other tumor | MMR | MMRP | N/A | −1.2784996 | −3.1266763 |
| 305256 + N_ | | | | Proficient | | | | |
| 286900 | | | | | | | | |
| K_11 + T_ | | Menengioma | Brain tumor | MMR | MMRP | N/A | −1.2792078 | −3.1188882 |
| 270648 + N_ | | | | Proficient | | | | |
| 27111 | | | | | | | | |
| K_90 + T_ | | Lymphoma | Hematological | MMR | MMRP | N/A | −1.282385 | −3.0724656 |
| 299554 + N_ | | | Cancer | Proficient | | | | |
| 299553 | | | | | | | | |
| K_5 + T_ | | Paranganglioma | Brain tumor | MMR | MMRP | N/A | −1.2831138 | −3.1314895 |
| 300238 + N_ | | | | Proficient | | | | |
| 245676 | | | | | | | | |
| K_6 + T_ | | Neuroblastoma | Other tumor | MMR | MMRP | N/A | −1.2856908 | −3.125563 |
| 274243 + N_ | | | | Proficient | | | | |
| 27111 | | | | | | | | |
| K_3 + T_ | | Sarcoma | Other tumor | MMR | MMRP | N/A | −1.2879304 | −3.129095 |
| 285202 + N_ | | | | Proficient | | | | |
| 285200 | | | | | | | | |
| K_3 + T_ | | Sarcoma | Other tumor | MMR | MMRP | N/A | −1.2907824 | −3.1295898 |
| 285201 + N_ | | | | Proficient | | | | |
| 285200 | | | | | | | | |
| K_32 + T_ | | CNS tumor | Brain tumor | MMR | MMRP | N/A | −1.2967652 | −3.0969994 |
| 276054 + N_ | | | | Proficient | | | | |
| 274026 | | | | | | | | |

TABLE 7
Immunotherapy Samples

| | | | | | | | | TMB | |
|---|---|---|---|---|---|---|---|---|---|
| Sample | Donor | Tissue_type | Tissue_category | Response | | MMR | MSI_Promega | (Mut/mb) | |
| D1243 | MMR100 | GBM | Brain tumor | Responder | | CMMRD | MSS | 496.24 | |
| D1410 | MMR111 | GBM | Brain tumor | Non-responder | | CMMRD | MSS | 318.06 | |
| D1423_1 | MMR120 | GBM | Brain tumor | Responder | | CMMRD | MSS | 699.5 | |
| D1577 | MMR139 | GBM | Brain tumor | Non-responder | | CMMRD | MSI-L | 336.78 | |
| D1755 | MMR111 | GI | GI tumor | Responder | | CMMRD | MSI-H | 81.98 | |
| | | Adenocarcinoma | | | | | | | |
| D1762 | MMR125 | GI | GI tumor | Responder | | CMMRD | MSI-H | 5.46 | |
| | | Adenocarcinoma | | | | | | | |
| D1763_1 | MMR117 | GBM | Brain tumor | Responder | | CMMRD | MSI-L | 294.02 | |
| D1805_2 | MMR101 | GBM | Brain tumor | Responder | | CMMRD | MSS | 368.32 | |
| D1806 | MMR128 | GBM | Brain tumor | Responder | | CMMRD | MSS | 296.9 | |
| JZ | MMR140 | Anaplastic | Brain tumor | Non-responder | | CMMRD | N/A | 295.82 | |
| | | Astrocytoma | | | | | | | |
| M1268T2 | MMR1268 | Anaplastic | Brain tumor | Non-responder | | CMMRD | N/A | 9 | |
| | | Astrocytoma | | | | | | | |
| MD1260T7 | MMR1260 | Anaplastic | Brain tumor | Non-responder | | Lynch | N/A | 7 | |
| | | Astrocytoma | | | | | | | |
| MD1273T25 | MMR1273 | GBM | Brain tumor | Responder | | CMMRD | N/A | 272 | |
| MD1281T1 | MMR1281 | GBM | Brain tumor | Non-responder | | Lynch | N/A | 13 | |
| MD1301T2 | MMR1301 | GI | GI tumor | Responder | | Lynch | N/A | 21 | |
| | | Adenocarcinoma | | | | | | | |
| MD1307T1 | MMR1307 | GBM | Brain tumor | Non-responder | | CMMRD | N/A | 7 | |
| MD1341T3 | MMR1341 | GBM | Brain tumor | Responder | | CMMRD | N/A | 282 | |
| MD152T2 | MMR152 | GBM | Brain tumor | Responder | | CMMRD | N /A | 184.58 | |
| (JG) | | | | | | | | | |
| MD1308T3 | MMR1308 | AML | Hematological | Non-responder | | CMMRD | N/A | 8.22 | |
| | | | cancer | | | | | | |
| MD1323T3 | MMR1323 | GBM | Brain tumor | Responder | | CMMRD | N/A | 689.9 | |
| MD1363T3 | MMR1363 | GBM | Brain tumor | Non-responder | | CMMRD | N/A | 12.92 | |
| MD1460T1 | MMR1460 | GBM | Brain tumor | Responder | | | N/A | 911.92 | |
| MD1468T1 | MMR1468 | GBM | Brain tumor | Responder | | | N/A | 200.02 | |
| MD140T11 | MMR140 | Liver Metastasis | Other tumor | Responder | | | N/A | 324.84 | |
| MD190T2 | MMR190 | GBM | Brain tumor | Non-responder | | | MSI-L | 371.1 | |
| MD139T2 | MMR139 | GBM | Brain tumor | Non-responder | | | MSI-L | 15.74 | |
| (D1578) | | | | | | | | | |
| MD66T13 | MMR66 | GBM | Brain tumor | Non-responder | | | N/A | 33.46 | |
| MD1467T2 | MMR1467 | GBM | Brain tumor | Responder | | | N/A | 451.8 | |
| MD1485T1 | MMR1485 | GBM | Brain tumor | Responder | | | N/A | | |
| MD1456T1 | MMR1456 | GI | GI tumor | Responder | | | N/A | 324.84 | |
| | | Adenocarcinoma | | | | | | | |
| MD1456T2 | MMR1456 | GBM | Brain tumor | Responder | | | N/A | 352.12 | |
| MD1385T2-2 | MMR1385 | GBM | Brain tumor | Responder | | | N/A | 287.02 | |
| MD1337T2 | MMR1337 | GI | GI tumor | Responder | | | N/A | 371.44 | |
| | | Adenocarcinoma | | | | | | | |
| | | | MMRDness | POLEness | MMRDness | POLEness | Immunotherapy | | |
| RRD Type | Deceased | Survival | (Genome) | (Genome) | (Exome) | (Exome) | Agent | Genome? | Exome? |
| MMRD & PPD | 0 | 3.94 | −1.07 | −2.76 | NA | NA | Nivolumab | Y | Y |
| MMRD & PPD | 1 | 1.72 | −0.97 | −2.72 | −1.14 | −2.57 | Pembrolizumab | Y | Y |
| MMRD & PPD | 1 | 0.09 | −1.01 | −2.96 | NA | NA | Nivolumab | Y | N |
| MMRD & PPD | 1 | 0.1 | −1.06 | −2.95 | −1.04 | −2.76 | Nivolumab | Y | Y |
| MMRD & PPD | 0 | 1.72 | −0.76 | −2.35 | −0.78 | −2.11 | Pembrolizumab | Y | Y |
| MMRD & PPD | 0 | 3.45 | −0.80 | −2.87 | −0.86 | −2.75 | Pembrolizumab | Y | Y |
| MMRD & PPD | 1 | 0.26 | −0.96 | −2.74 | NA | NA | Nivolumab | Y | N |
| MMRD & PPD | 1 | 0.84 | −0.90 | −2.63 | NA | NA | Nivolumab | Y | N |
| MMRD & PPD | 1 | 0.33 | −0.85 | −2.83 | NA | NA | Nivolumab | Y | Y |
| MMRD | 1 | 2.09 | −1.05 | −3.05 | −1.15 | −2.90 | Nivolumab | Y | Y |
| MMRD | 1 | 0.1 | −1.07 | −3.09 | −1.06 | −2.94 | Nivolumab | Y | Y |
| MMRD | 1 | 0.08 | −0.92 | −3.07 | −1.02 | −2.96 | Nivolumab | Y | Y |
| MMRD & PPD | 0 | 2.47 | −1.04 | −2.18 | −1.07 | −1.96 | Nivolumab | Y | Y |
| MMRD | 1 | 0.46 | NA | NA | −0.88 | −2.87 | Nivolumab | Y | Y |
| MMRD | 0 | 3.4 | −0.72 | −2.90 | −0.85 | −2.85 | Pembrolizumab | Y | Y |
| MMRD | 1 | 0.38 | −0.88 | −3.08 | −0.98 | −2.96 | Nivolumab | Y | Y |
| MMRD & PPD | 0 | 2.15 | −1.03 | −2.80 | −1.13 | −2.62 | Nivolumab | Y | Y |
| MMRD & PPD | 1 | 1.81 | −1.20 | −3.10 | −1.09 | −2.40 | Nivolumab | Y | Y |
| MMRD | 1 | 0.31 | −1.06 | −3.07 | −1.08 | −2.90 | Nivolumab | Y | Y |
| MMRD | 0 | 2.46 | −1.13 | −2.88 | −1.11 | −2.69 | Nivolumab | Y | Y |
| MMRD | 1 | 0.38 | −1.18 | −3.10 | −1.19 | −2.95 | Nivolumab | Y | Y |
| MMRD & PPD | 0 | 0.99 | −1.04 | −2.96 | −1.03 | −2.79 | Nivolumab | Y | Y |
| MMRD | 0 | 0.74 | NA | NA | −1.13 | −2.76 | Nivolumab | N | Y |
| MMRD | 0 | 2.09 | NA | NA | −1.05 | −2.35 | Nivolumab | N | Y |
| MMRD & PPD | 1 | 0.98 | NA | NA | −1.07 | −2.28 | Nivolumab | N | Y |
| MMRD | 1 | 0.1 | NA | NA | −1.09 | −2.92 | Nivolumab | N | Y |
| MMRD | 0 | 3 | NA | NA | −1.13 | −2.94 | Pembrolizumab | N | Y |
| MMRD | 0 | 0.66 | NA | NA | −1.04 | −2.48 | Nivolumab | N | Y |
| MMRD | 0 | 0.59 | NA | NA | −0.98 | −2.58 | Nivolumab | N | Y |
| MMRD | 0 | 4.02 | NA | NA | −0.93 | −2.43 | Nivolumab | N | Y |
| MMRD | 0 | 4.02 | NA | NA | −1.00 | −2.64 | Nivolumab | N | Y |
| MMRD & PPD | 0 | 1.8 | NA | NA | −1.11 | −2.63 | Nivolumab | N | Y |
| PPD | 0 | 1.93 | NA | NA | −1.14 | −2.83 | Nivolumab | N | Y |

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for characterizing a biological sample from a subject, the method comprising calculating MS-sig1 (MMRDness score) and MS-sig2 (POLEness score) that reflect the prevalence of single base insertions and deletions, respectively, in the sample, wherein an increase in either score compared to a reference sample identifies the biological sample as replication repair deficient.

2. The method of claim 1, wherein the score further characterizes the biological sample as mismatch repair proficient (MMRP) if the MMRDness score is not increased, mismatch repair deficient (MMRD) if the MMRDness score is increased, polymerase proof-reading deficient (PPRD/PPD) if the POLEness score is increased, or MMRD&PPD if both the MMRDness and PONEness scores are increased.

3. A method for selecting therapy for a subject having a cancer or tumor, the method comprising calculating a POLEness score for a biological sample obtained from the subject, wherein an increased POLEness score compared to a reference sample selects immune checkpoint inhibitor therapy for the subject, and a decreased or unincreased POLEness score compared to a reference sample indicates that immune checkpoint inhibitor therapy is not appropriate for the subject.

4. The method of claim 3, wherein the immune checkpoint inhibitor immune therapy comprises administering an immune checkpoint inhibitor to the subject.

5. The method of claim 4, wherein the immune checkpoint inhibitor is a PD-1/PD-L1 inhibitor.

6. The method of claim 4, wherein the immune checkpoint inhibitor comprises an antibody or a fragment thereof a selected from the group consisting of nivolumab, pembrolizumab, atezolizumab, durvalumab, and/or avelumab.

7. The method of claim 1, wherein the calculating comprises analyzing sequence data obtained from the biological sample.

8. The method of claim 3, further comprising administering the immune checkpoint inhibitor therapy to the subject with the increased POLEness score.

9. A method for characterizing a cancer or tumor in a subject, the method comprising analyzing sequencing data obtained from a biological sample of the subject to identify microsatellite signatures in the sequencing data and
a) using the identified microsatellite signatures to calculate an MMRDness score, and identifying the cancer as a mismatch repair deficiency (MMRD) cancer if the MMRDness score is elevated compared to a reference sample;
b) using the identified microsatellite signatures to calculate a POLEness score, and identifying the cancer as a polymerase proofreading deficiency (PPD) cancer if the POLEness score is elevated compared to a reference sample; or
c) using the identified microsatellite signatures to calculate a POLEness score and an MMRDness score, and identifying the cancer or tumor as a replication repair deficiency (RRD) cancer or tumor if the POLEness score and/or the MMRDness score is elevated compared to a reference sample.

10. The method of claim 3, wherein the cancer or tumor is a lymphoma, glioma, brain cancer, endometrial cancer, stomach cancer, or colorectal cancer.

11. The method of claim 1, wherein the subject has Lynch syndrome.

12. The method of claim 7, wherein the sequence data is ultra-low pass coverage sequencing data.

13. The method of claim 12, wherein the sequence coverage is nonzero and is less than 1×.

14. The method of claim 13, wherein the sequence coverage is between about 0.1× and about 0.5×.

15. The method of claim 7, wherein the sequence data is whole-exome or whole genome sequence data.

16. The method of claim 7, wherein the sequencing data is obtained by sequencing cell free DNA.

17. The method of claim 1, wherein the subject has germline constitutional mismatch repair deficiency.

18. A method of treating a subject having a cancer or tumor, the method comprising administering an immune checkpoint inhibitor to the subject, wherein the subject is selected by calculating a POLEness score for a biological sample obtained from the subject, wherein an increased POLEness score compared to a reference sample selects immune checkpoint inhibitor therapy for the subject.

19. The method of claim 18, wherein the immune checkpoint inhibitor is a PD-1/PD-L1 inhibitor.

20. The method of claim 19, wherein the immune checkpoint inhibitor comprises an antibody or a fragment thereof.
